SEQUENCE LISTING (28 sequences)

SEQ ID NO: 1 - DNA, 5 bp, Artificial Sequence
  Synthetic Construct
attcc

SEQ ID NO: 2 - DNA, 24 bp, Artificial Sequence
  Sat2-24nt LNA (Exiqon)
attccattcagattccattcgatc

SEQ ID NO: 3 - DNA, 54 bp, Artificial Sequence
  Sat2-59nt Forward strand (Invitrogen)
antccattcgggtccattcgatgatgatcacactggatttcattccataattct

SEQ ID NO: 4 - DNA, 59 bp, Artificial Sequence
  Sat2-59nt Reverse compliment (Invitrogen)
cgaatagaattatggaatgaaatccagtgtgatcatcatcgaatggacccgaatggant

SEQ ID NO: 5 - DNA, 24 bp, Artificial Sequence
  Sat2-160bp PCR Forward primer
catcgaatggaaatgaaaggagtc

SEQ ID NO: 6 - DNA, 22 bp, Artificial Sequence
  Sat2-160bp PCR Reverse primer
ttgactgcaatcatccaatggt

SEQ ID NO: 7 - DNA, 26 bp, Artificial Sequence
  Sat2_1q12 Forward primer
ggaaccgaatgaatcctcattgaatg

SEQ ID NO: 8 - DNA, 27 bp, Artificial Sequence
  Sat2_1q12 Reverse primer
atgattccattcgattcaatgttccat

SEQ ID NO: 9 - DNA, 26 bp, Artificial Sequence
  Sat2_7 Forward primer
attcgattccattcgatgatgattcc

SEQ ID NO: 10 - DNA, 26 bp, Artificial Sequence
  Sat2_7 Reverse primer
ggaaccgaatgaatcctcattgaatg

SEQ ID NO: 11 - DNA, 20 bp, Artificial Sequence
  Sat3 (Invitrogen)
ccattccattccattccatt

SEQ ID NO: 12 - DNA, 59 bp, Artificial Sequence
  HuAlphaSat (59 mer)
ccttttgatagagcagttttgaaacactctttttgtagaatctgcaagtggatatttgg

SEQ ID NO: 13 - DNA, 33 bp, Artificial Sequence
  HuAlu (33 mer)
cccaaagtgctgggattacaggcgtgagccacc

SEQ ID NO: 14 - DNA, 1352 bp, Homo sapiens
gatcatcatcgaatggacccgaatggaatcaatcatccaacggaagctaatggaatcaacatcgaatgaatcgaatggaaacaccatcgaattgaaacgaatggaattctcatgaaattgaaatggatggactcgtcatcgaatggattcgaatggaatcatcgaataaaattgattgaaatcatcatcaagtggaatcgaatggtatcattgaatggaatcgaatggaatcatcagatggaaatgaattgaatcgtcatagaatggaatcgaatggattcattgaatggaatcagatggaatcatcgaatggactggaatggaatcattgaatggactcgaaaggaatcatcatcaaatggaaccgaatgaatcctcattgaatggaaatgaaaggggtcatcatctaatggaatcgcatggaatcatcatcaaatggaatcgaatggaatcatcatcaaatggcaatctaatggaatcattgaacagaattgaatggaatcgtcatcgaatgaattgaatgcaatcatcgaatggtctcgaatggaatcatcttctaatggaaaggaatggaatcatcgcatagaatcgaatggaattatcatcgaatggaatcgaatggtatcaaacggaaaaaaacggaattatcgaatggaatcgaagagaatcttcgaacggacccgaatggaatcatctaatggaatggaatggaataatccactggactcgaatgcaatcatcatcgaatggaatggaatggaatcatcgaatggactcgaatggatggaacattgaatcgaatggaatcatcaatcggatggaaacgaatggaatcatcatcgaatggaaatgaaaggagtcatcatctaatggaattgcatggaatcatcataaaatggaatcgaatggaatcaacatcaaatggaatcaaatggaatcattgaacggaattgaatggaatcgtcatcgaatgaattgactgcaatcatccaatggtcgcgaatggaatcatcttcaaatggaatggaatggaatcatcgcatagaatcgaatggaattatcatcgaatggaatcgaatggaatcaacatccaacggaaaaaaacggaattatcgaatggaatcgaagagaatcatcgaatggacccgaatggaatcatctaatggaatggaatggaataatccatggactcgaatgcaatcatcatcgaatggaatcgaatggaatcatcgaatggactcgaatggaataatcattgaacggcatcgaatggaatcatcatcggatggaaatgaatggaatcatcatcgaatggaatcgaatagaattatggaatgaaatccagtgtgatc

SEQ ID NO: 15 - DNA, 160 bp, Homo sapiens
catcgaatggaaatgaaaggagtcatcatctaatggaattgcatggaatcatcataaaatggaatcgaatggaatcaacatcaaatggaatcaaatggaatcattgaacggaattgaatggaatcgtcatcgaatgaattgactgcaatcatccaatggt

SEQ ID NO: 16 - DNA, 134 bp, Homo sapiens
aattcatttgaagacaattccattccattcaataccaattgatggttatttttgattccatttgatgatgattacattccatttcatcataattccattcgattccactcgagattccattcgattccattcaa

SEQ ID NO: 17 - DNA, 75 bp, Homo sapiens
cgaatgaatgagtccatccatttcaatttcatgataattccattcgtttcaattcgatggtgtttccattcgatt

SEQ ID NO: 18 - DNA, 89 bp, Homo sapiens
ttcattcgattcatttgatgatgattcatgcgcgattcattagatgacccctttcatttccttcaatggaggattccattcggttccat

SEQ ID NO: 19 - DNA, 551 bp, Homo sapiens
  misc_feature (377)..(384): n is a, c, g, or t
atcatcaaatggaatcgaatggaatcatctaatgaacttgaaaggaatcatcatcaaatgaagtcaaatggaataaccaaatcgacatgaatggaatcatcattgaatagaatcgaatggattcattaaatgggctagaatggaatcatctgtgaacggattcaaagggaatcatcatcgaatggaatcgaatggaaatgtcaaccaattgaatcatcatggaattgaatcgaatggctcactgtcgaatcaaatcaaatggaatcaatcaatccgtaatggaattgaatggaaacaatgaaaggaattgaatggaatcatcattgaatggaacccgaatgaaatcatcaacgaatggaatccaatgtaatcatcannnnnnnnggtaagcacttaaggaatcattatccgaatggaattaaatgaaatctataatccgaatggaatccgaatggaatcatcatcgaatggaatggaatggaaacgttattgaatggaattaaatgaaattaccgaaatgaatgaaatggattgatcattaaagggaatta

SEQ ID NO: 20 - DNA, 735 bp, Homo sapiens
attccattcgattccattcgatgatgattcattcggttccatgcaatgatgatttcatttgtttccatttgatgatgattccattggataccatttgatgatgattccatttgataccattcgatgatgattccattcgattccattcaatgacgattccattcgattccattcaatgaagagtccattggtttccatttcatgattccattcgactccatttgatgatgattccttcggaatccattcgatgattccattcgattccattcgatgatgattccattcgattccgttcaatgattattccattcgagtccattcgatgattccattcgattccattcgatgatgattgcattcgagtccatggattcttccatttcattccattagatgattccattcgagtccgttcgatgattctcttcgattccattcgataattccgtttgattccgtttgatgttgattccattggagtccattcgatgataattccattcgattctatgcgatgattccattccattccatttggagatggttccattcgagaccattcgttgatggcattcaattcattcgatgacgattccattcaattccgttcaatgattccattagattccatttcatgatgattccattcgattccatttgatgatgattccatgagattccattagatgatgactcctttcatttccattcaatgaggattcattcggttcc

SEQ ID NO: 21 - DNA, 406 bp, Homo sapiens
attccattcgattccattcgatgatgattcattcggttccatggattattccattccattccattagatgattccattcgggtccattcgatgattctcttcgagtccattcgataattccgtttttttccgtttgatgttgattccattcgattccattcgatgataattccattcgattctatgcgatgattccatttcattccatttgaagatgattccattcgagaccattcgatgattgcattcaattcattcgatgacgattccattcaattccgttcaatgattccatttgattccatttgatgttgattccattcgattccattttatgatgattccatgcaattccattagatgatgactcctttcatttccattcaatgaggattcattcggttcc

SEQ ID NO: 22 - DNA, 710 bp, Homo sapiens
ggaaccgaatgaatcctcattgaatggaaatgaatggaatcatcatagaatggaatcgaatggattcattgaatggaatcagatggaatcattgaatgggcttcaatggaatcattcaatggactcaaatggaatcattattgaatggaattcaatggaatcatcgaatggtcacgaatggaatcatcatcgaatggaatcgaatttaatcatcaagtggaatcaaatggaatcatcatcgaatggaatcgaatagaatcggcattgaatagaatcgaatggaatcatcatcaatggaatcgaatggaattttcttcaaatggattcgaatgcaaacatcatcgaatagaatcaaatgggatcatcaaatgaaactgaatggaatcatcatcaaaacgaatcaaaataaaacagagaatggaatccaatggaatcatcgaatggaatcaaatggaatcatcattgaatggactcgaatggagtcatcatcgaatggaatcgaatggaataatccacggacccgaatgcaatcatcatcgtatagaatcgaatggaatcatcgaatggactcgaaaggaataatcattgaacggaatcgaatggaatcatcatcggatggaaacgaatggaatcatcatcgaatggaaatgaaaggagtcatcatctaatggaattgcatggaatcatcatcgaatggaatcgaatggaat

SEQ ID NO: 23 - DNA, 609 bp, Homo sapiens
attccattcgattccattcgatgatgattccattagcttccgttggatgatgattccattcgggtccattcgatgatgatcacactggatttcattccataattctattcgattccattcgatgatgattccattcatttccatccgatgatgattccattcgattccgttcaatgattattccattcgagaccattcgatgattccattcgattccattcgatgatgattgcattcgagtccatggattattccattccattccattagatgattccattcgggttcattcgatgattctcttcgattccatacgagaattctgtttttttccgtttgatgttgattccattcgattccattcgatgataattccattcgattctatgcgatgattccattccattccatttgaagatggttccattcgagaccattcgttgattgcattcaattcattcgatgacgattccattcaattccgttcagtgattccattagattccatttgatgatgattccattcgattccatttgatgatgattccatgcgattccattagatgatgactcctttcatttccattcaatgaggattcattcggttcc

SEQ ID NO: 24 - DNA, 132 bp, Homo sapiens
ccattccattttatttgataatgattccattcgatgatgattccattcgattccattcgatgatgattccattcgattccattcaatgatgattccattcgattccattcaatgatgattcgtttcgatcca

SEQ ID NO: 25 - DNA, 277 bp, Homo sapiens
tggatcgaaacgaatcatcattgaatggaatcgaatggaatcatcattgaatggaatcgaatggaatcatcatcgaatggaatcgaatggaatcatcatcgaatggaatcattatcaaataaaatggaatggaatcattgaatggaatcgaatggaatcatcatcgaatggaatcgaacggtatcatcaaatggaatcgaatggaatcatcattgaatagaatcgaatggaatcatcgaatggaactgaatggaatcatcatcgaatggaatcgaat

SEQ ID NO: 26 - DNA, 349 bp, Homo sapiens
tggatcgaaacgaatcatcattgaatggaatcgaatggaatcatcattgaatggaatcgaatggaatcatcatcgaatggaatcgaatggaatcatcatcgaatggaatcattatcaaataaaatggaatggaatcattgaatggaatcgaatggaatcatcatcgaatggaatcgaacggtatcatcaaatggaatcgaatggaatcatcattgaatagaatcgaatggaatcatcgaatggaactgaatggaatcatcattgaatggaatcgaatggaatcaccgaatggaatcgaatggaatcatcaaatggaatcgaatggaatcatcatcgaatggaatcgaat

SEQ ID NO: 27 - DNA, 129 bp, Homo sapiens
atcgaaacgaatcatcattgaatggaatcgaatggaatcatcattgaatggaatcgaatggaatcatcatcgaatggaatcgaatggaatcatcatcgaatggaatcattatcaaataaaatggaatgg

SEQ ID NO: 28 - DNA, 134 bp, Homo sapiens
tggatcgaaacgaatcatcattgaatggaatcgaatggaatcatcatcgaatggaatcgaatggaatggactcaaatggaatgtgctcgaatggaatggaatcgaatggattgatatcaaataaaatggaatgg

(19) United States
(12) Patent Application Publication
(10) Pub. No.: US 2014/0213475 A1
(43) Pub. Date: Jul. 31, 2014

(54) METHODS OF DIAGNOSING CANCER USING EPIGENETIC BIOMARKERS

(71) Applicants: Jeanne B. Lawrence, Mapleville, RI (US); Lisa Hall, Sudbury, MA (US); Meg Byron, Sterling, MA (US); Dawn M. Carone, Auburn, MA (US)

(72) Inventors: Jeanne B. Lawrence, Mapleville, RI (US); Lisa Hall, Sudbury, MA (US); Meg Byron, Sterling, MA (US); Dawn M. Carone, Auburn, MA (US)

(73) Assignee: University of Massachusetts, Boston, MA (US)

(21) Appl. No.: 14/232,552

(22) PCT Filed: Jul. 16, 2012
(86) PCT No.: PCT/US2012/046959

§ 371 Date: Mar. 11, 2014

Related U.S. Application Data
(60) Provisional application No. 61/507,937, filed on Jul. 14, 2011.

(51) Int. Cl. C12Q 1/68; G01N 33/50; G01N 33/574

(52) U.S. Cl. CPC C12Q 1/6886; G01N 33/57496; G01N 33/5017; G01N 33/5011
USPC 506/9; 435/7.92; 435/6.14; 435/6.11; 435/6.12; 435/7.1; 435/7.4; 435/6.13

(57) ABSTRACT

The invention features methods of diagnosing cancer in a mammal (e.g., a human) by detecting a biomarker selected from a satellite II ribonucleic acid (RNA) molecule, a cancer-associated polycomb group (CAP) body, a cancer-associated satellite transcript (CAST) body, and UbH2A. Also featured is a method for identifying an agent for treating cancer in a mammal by contacting a cancer cell having a biomarker selected from a CAP body, a CAST body, and a satellite II RNA molecule with a test agent and determining whether the test agent reduces the level of the biomarker in the cancer cell. Other inventions featured are a method for determining whether a chemotherapeutic agent increases epigenetic imbalance of a cell and a method for detecting epigenetic imbalance by determining a copy number of a satellite II DNA locus at chromosome 1q12 in a cell.

### CROSS-REFERENCE TO RELATED APPLICATIONS

This application claims benefit of U.S. Provisional Application No. 61/507,937, filed Jul. 14, 2011, the contents of which are hereby incorporated by reference in their entirety.

### STATEMENT AS TO FEDERALLY FUNDED RESEARCH

This invention was made with government support under grant number R37 GM053234 awarded by the NIH. The government has certain rights in this invention.

### BACKGROUND OF THE INVENTION

Currently many efforts are underway to identify new “biomarkers” for cancer, which will facilitate more accurate diagnosis, classification, and therapeutic responses to cancer. While there are many studies of specific changes in proteins, mRNAs, microRNAs, or DNA methylation in cancer, studies using repeat RNAs were essentially unknown, since they are usually thought of as transcriptionally inert genomic elements. It is generally not considered that specific types of repeats may be expressed in cancer, despite the abundant literature suggesting that they are commonly hypomethylated during carcinogenesis. In fact, almost all genomic studies mask out the repeat sequences from their analyses, therefore precluding the possibility of discovering aberrations in repeat expression. About half of the human genome encodes repeat sequences of varying sorts, the function of which is largely unknown.

Much attention has been focused recently on the silencing of tumor suppressor genes in cancers by hypermethylation (epigenetics) instead of DNA mutation. However, these studies recognize a major paradox: hypermethylation often occurs in the context of broader genomic hypomethylation, including at centric/pericentric satellites. Despite its abundance, satellite II (Sat II) repeats found within the pericentromere of many chromosomes have no known function in normal cells or in disease. In fact several studies have noted hypomethylation of Sat II in cancer, but this is not presumed to have a functional impact, but rather may be considered secondary to the clearer functional implications of tumor suppressor gene hypermethylation and silencing. The hypermethylation of some regions of the nucleus in the same cell exhibiting widespread hypomethylation suggests a dramatic imbalance in the epigenome, which may not be explained by simple overexpression or reduction in a biomarker or regulatory factor.

Polycomb group (PcG) proteins are a family of master epigenetic regulators that control most early developmental pathways, primarily through repressive chromatin modifications, and are also involved in the formation and maintenance of constitutive peri/centric satellite heterochromatin. Polycomb repressive complex 2 (PRC2) includes the EZH2 protein, which introduces trimethylation of histone H3 lysine 27, whereas polycomb repressive complex 1 (PRC1) includes BMI-1, RING1B and Phc-1, and promotes histone ubiquitination, DNA compaction and other modifications. In mammalian cells, prominent PcG bodies have previously been described; however, they are widely considered to be part of normal nuclear structure and are currently studied as such, although studies are primarily conducted on cancer cell lines, which are presumed to reflect normal nuclear structure. BMI-1 is a key component of PRC1 linked to cell proliferation, senescence, self-renewal and tumor suppressor gene regulation (Ink4a/Arf), and is over-expressed in several tumor types. Although BMI-1 over-expression is linked to cancer progression and prognosis, its role is complex and currently unresolved, despite intense study.

There still exists a need for cancer biomarkers that can be used for surveillance, recognition and proper classification of different cancers and for designing/evaluating therapeutic interventions.

### SUMMARY OF THE INVENTION

The invention relates to a first method of diagnosing, or providing a prognostic indicator of, cancer (e.g., metastatic cancer or a cancer selected from breast cancer (e.g., adenocarcinoma, ductal carcinoma, lobular carcinoma, metaplastic carcinoma, and papillary carcinoma), ovarian cancer (e.g., adenocarcinoma and carcinoma (metastatic)), Wilms tumor, multiple myeloma, brain cancer (e.g., glioblastoma), kidney cancer (e.g., renal cell carcinoma), lung cancer (e.g., squamous cell carcinoma), fibrosarcoma, prostate cancer (e.g., adenocarcinoma), stomach cancer (e.g., adenocarcinoma and gastrointestinal stromal tumor (GIST)), thyroid cancer (e.g., papillary carcinoma), bone cancer, colon cancer (e.g., adenocarcinoma), pancreatic cancer (e.g., serous cystadenoma (benign)), or cervical cancer) in a mammal (e.g., a human) by detecting at least one (or two or more) biomarker(s) selected from a satellite II ribonucleic acid (RNA) molecule, a cancer-associated polycomb group (CAP) body, and a cancer-associated satellite transcript (CAST) body in a sample from the mammal. In several embodiments, an increase in the level of expression of the satellite II RNA molecule in a cell of the sample, relative to the level of expression of the satellite II RNA molecule in a normal cell, or abnormal nuclear compartmentalization of the CAP body or the CAST body in a cell of the sample, relative to nuclear compartmentalization of the CAP body or the CAST body in a normal cell, indicates the sample includes at least one (or two or more) cancer cell(s). In another embodiment, the method includes detecting the level of expression of the CAP or CAST body and the satellite II ribonucleic acid (RNA) molecule in the sample.

The invention also relates to a second method for identifying an agent for the treatment of a cancer (e.g., metastatic cancer or a cancer selected from breast cancer (e.g., adenocarcinoma, ductal carcinoma, lobular carcinoma, metaplastic carcinoma, and papillary carcinoma), ovarian cancer (e.g., adenocarcinoma and carcinoma (metastatic)), Wilms tumor, multiple myeloma, brain cancer (e.g., glioblastoma), kidney cancer (e.g., renal cell carcinoma), lung cancer (e.g., squamous cell carcinoma), fibrosarcoma, prostate cancer (e.g., adenocarcinoma), stomach cancer (e.g., adenocarcinoma and gastrointestinal stromal tumor (GIST)), thyroid cancer (e.g., papillary carcinoma), bone cancer, colon cancer (e.g., adenocarcinoma), pancreatic cancer (e.g., serous cystadenoma (benign)), or cervical cancer) in a mammal (e.g., a human) by contacting a cancer cell that includes at least one (or two or more) biomarker(s) selected from a cancer-associated polycomb group (CAP) body, a cancer-associated satellite transcript (CAST) body, or a satellite II RNA molecule with a test agent and determining whether the test agent reduces the level of the biomarker. In an embodiment, the method includes detecting a reduction in the formation of the CAP body or CAST body, or a reduction in expression of the satellite II RNA molecule, in the cancer cell following contact with the test agent, in which a reduction in the level of the biomarker in the cancer cell, relative to the level of the biomarker in a cancer cell not contacted with the test agent, indicates that the test agent is suitable for the treatment of the cancer.

The invention also relates to a third method for determining whether a chemotherapeutic agent increases epigenetic imbalance in a cell(s) of a mammal (e.g., a human) by contacting a sample that includes the cell(s) with a chemotherapeutic agent and determining a level of one (or two or more) biomarker(s) selected from a cancer-associated polycomb group (CAP) body, a cancer-associated satellite transcript (CAST) body, and a satellite II RNA molecule in the cell. In an embodiment, an increase in the level of the biomarker(s) in the cell(s), relative to the level of the biomarker in a cell(s) not contacted with the chemotherapeutic agent, indicates that the chemotherapeutic agent increases epigenetic imbalance in the cell(s). In another embodiment, the increase in the level of the biomarker(s) indicates the chemotherapeutic agent increases a risk of cancer in the mammal (e.g., the increase in the level of the biomarker(s) indicates an increased risk the cancer will become more aggressive).

The invention also relates to a fourth method for diagnosing, or providing a prognostic indicator of, cancer (e.g., metastatic cancer or a cancer selected from breast cancer (e.g., adenocarcinoma, ductal carcinoma, lobular carcinoma, metaplastic carcinoma, and papillary carcinoma), ovarian cancer (e.g., adenocarcinoma and carcinoma (metastatic)), Wilms tumor, multiple myeloma, brain cancer (e.g., glioblastoma), kidney cancer (e.g., renal cell carcinoma), lung cancer (e.g., squamous cell carcinoma), fibrosarcoma, prostate cancer (e.g., adenocarcinoma), stomach cancer (e.g., adenocarcinoma and gastrointestinal stromal tumor (GIST)), thyroid cancer (e.g., papillary carcinoma), bone cancer, colon cancer (e.g., adenocarcinoma), pancreatic cancer (e.g., serous cystadenoma (benign)), or cervical cancer) in a mammal (e.g., a human) by detecting, in a cell present in a sample from the mammal, one or more of a change in the ubiquitination status of histone H2A, the presence of a biomarker selected from a mutant BRCA1 protein that exhibits an impaired ability to monoubiquitylate histone H2A, relative to wild-type BRCA1 protein, or a mutant BRCA1 gene that encodes the mutant BRCA1 protein, or an altered distribution of UbH2A or PRC1 complex, each of which is relative to a normal cell. In preferred embodiments, the change in histone H2A ubiquitination status is altered (e.g., unbalanced) distribution of ubiquitinated histone H2A (UbH2A) relative to a normal cell (e.g., an increase in UbH2A foci relative to UbH2A foci in a normal cell). In another embodiment, the altered distribution of UbH2A is caused by a perturbed distribution of PRC1 complex (or one or more proteins of the PRC1 complex or its associated proteins, such as BMI-1, RING 1B, Phc1, Phc2, CBX4, CBX8, RNF2, GLI1, MYC, CDKN2A, and HST2H2AC), which is known to mediate recruitment of UbH2A to heterochromatin.

The invention also relates to a fifth method for screening an agent for efficacy in a treatment of a cancer in a mammal (e.g., a human) by contacting the agent to either: a) a cell (e.g., a cancer cell) that includes a biomarker selected from a mutant BRCA1 protein that exhibits an impaired ability to monoubiquitylate histone H2A, relative to wild-type BRCA1 protein, or a mutant BRCA1 gene that encodes the mutant BRCA1 protein; or b) a cell (e.g., a cancer cell) that exhibits, as a biomarker, a decreased level of monoubiquitylated histone H2A, relative to, e.g., a wild-type BRCA1-expressing cell, and determining whether the agent increases the monoubiquitylation of histone H2A in the cell.

The invention also relates to a sixth method for determining whether a chemotherapeutic agent increases epigenetic imbalance in a cell (e.g., a non-cancer cell) of a mammal (e.g., a human) by contacting the cell with the chemotherapeutic agent and determining a level of monoubiquitylation of histone H2A as a biomarker in the cell. A determination that the chemotherapeutic agent decreases the level of monoubiquitylation of histone H2A in the cell, relative to a cell not contacted with the chemotherapeutic agent, indicates that the chemotherapeutic agent causes an increase in epigenetic imbalance and should not be administered to the mammal as a treatment of cancer.

The invention further relates to a seventh method for diagnosing, or providing a prognostic indicator of, cancer (e.g., metastatic cancer or a cancer selected from breast cancer (e.g., adenocarcinoma, ductal carcinoma, lobular carcinoma, metaplastic carcinoma, and papillary carcinoma), ovarian cancer (e.g., adenocarcinoma and carcinoma (metastatic)), Wilms tumor, multiple myeloma, brain cancer (e.g., glioblastoma), kidney cancer (e.g., renal cell carcinoma), lung cancer (e.g., squamous cell carcinoma), fibrosarcoma, prostate cancer (e.g., adenocarcinoma), stomach cancer (e.g., adenocarcinoma and gastrointestinal stromal tumor (GIST)), thyroid cancer (e.g., papillary carcinoma), bone cancer, colon cancer (e.g., adenocarcinoma), pancreatic cancer (e.g., serous cystadenoma (benign)), or cervical cancer) in a mammal (e.g., a human) by detecting, as a biomarker, the ubiquitination status of histone H2A and/or the distribution of a heterochromatic marker (e.g., ubiquitinated histone H2A (UbH2A), H3K27me, H3K9me2, HP1, H4K20me, loss of H3K4me, loss of H4Ac, DNA methylation (5-mC), and macroH2A) in a cell of the mammal. In an embodiment, the distribution of the heterochromatic marker is unbalanced (e.g., prominent foci of the heterochromatic marker (e.g., one or more of UbH2A, H3K27me, H3K9me2, HP1, H4K20me, loss of H3K4me, loss of H4Ac, DNA methylation (5-mC), and macroH2A) are apparent in a cell of the mammal suspected of being a cancer cell (e.g., within the same nucleus some regions exhibit prominent foci of the heterochromatic marker (e.g., one or more of UbH2A, H3K27me, H3K9me2, HP1, H4K20me, loss of H3K4me, loss of H4Ac, DNA methylation (5-mC), and macroH2A) and other regions exhibit little to no foci), but not in normal cells). In yet another embodiment, an unbalanced distribution of the heterochromatic marker can be determined upon visual detection using, e.g., a microscope, or using an automated system (e.g., quantification using an automated platform). The method can be performed using, e.g., chromatin immunoprecipitation (ChIP) or a ChIP sequence (ChIP-level. The presence of a cancer cell in the sample can be based upon the observation of a characteristic “patchy” (much less evenly distributed) pattern in the nucleus of the cell. Thus, the overall distribution of a heterochromatic marker (e.g., one or more of UbH2A, H3K27me, H3K9me2, HP1, H4K20me, loss of H3K4me, loss of H4Ac, DNA methylation (5-mC), and macroH2A) shows “imbalance” in the nucleus, which may impact a variety of other genes and regulator proteins (tumor supressors, oncogenes etc.) in the cell. In another embodiment, the unbalanced heterochromatic marker (e.g., one or more of UbH2A, H3K27me, H3K9me2, HP1, H4K20me, loss of H3K4me, loss of H4Ac, DNA methylation (5-mC), and macroH2A) is present on on Sat II 1q12 and/or 16q11. In still other embodiments, detection of an imbalance of a heterochromatic marker (e.g., one or more of UbH2A, H3K27me, H3K9me2, HP1, H4K20me, loss of H3K4me, loss of H4Ac, DNA methylation (5-mC), and macroH2A) in the nucleus indicates the likelihood of a cancer cell (e.g., a cell that exhibits uncontrolled growth, metastasis, drug resistance, etc.) in the sample or the likelihood that a cell in the patient will progress to a cancer state (e.g., an aggressive cancer state). In another embodiment, the method is performed using a sample that includes at least one cell from a subject at risk from cancer. In a preferred embodiment, the method includes the use of a microarray to detect the ubiquitin status of H2A and/or the distribution of the heterochromatic marker (e.g., one or more of UbH2A, H3K27me, H3K9me2, HP1, H4K20me, loss of H3K4me, loss of H4Ac, DNA methylation (5-mC), and macroH2A) in a cell of the subject. In yet another embodiment, the detection of the heterochromatic marker (e.g., one or more of UbH2A, H3K27me, H3K9me2, HP1, H4K20me, loss of H3K4me, loss of H4Ac, DNA methylation (5-mC), and macroH2A) in a cell of a subject, relative to the distribution of the heterochromatic marker (e.g., one or more of UbH2A, H3K27me, H3K9me2, HP1, H4K20me, loss of H3K4me, loss of H4Ac, DNA methylation (5-mC), and macroH2A) in a normal cell, is determined using an antibody that specifically binds to the heterochromatic marker (e.g., one or more of UbH2A, H3K27me, H3K9me2, HP1, H4K20me, loss of H3K4me, loss of H4Ac, DNA methylation (5-mC), and macroH2A). In another embodiment, detection of a “patchy” distribution of the heterochromatic marker (e.g., one or more of UbH2A, H3K27me, H3K9me2, HP1, H4K20me, loss of H3K4me, loss of H4Ac, DNA methylation (5-mC), and macroH2A), as seen by, e.g., ChIP, in a cell of a subject, relative to the distribution of the heterochromatic marker (e.g., one or more of UbH2A, H3K27me, H3K9me2, HP1, H4K20me, loss of H3K4me, loss of H4Ac, DNA methylation (5-mC), and macroH2A) in a normal cell, indicates the mammal has a cancer.

The invention also relates to a eighth method for detecting epigenetic imbalance in a cell present in a sample from a mammal (e.g., a human) by determining a copy number of a satellite II DNA locus at chromosome 1q12 in the cell or the level of polycomb proteins on a satellite II DNA locus at chromosome 1q12 in the cell. In an embodiment, an increase in the copy number of, or the amount of polycomb protein on, the satellite II DNA locus at chromosome 1q12 in the cell indicates the cell has epigenetic imbalance. In another embodiment, detection of the epigenetic imbalance in the cell indicates an increased risk of cancer in the mammal.

The invention also relates to a ninth method for diagnosing, or providing a prognostic indicator of, immunodeficiency, centromeric region instability, and facial anomalies syndrome (ICF), which is a rare chromosome breakage disease caused by mutations in the methyl transferase DNMT3B enzyme. The diagnostic characteristics of ICF are agammaglobulinemia with B cells as well as DNA rearrangements targeted to the centromere-adjacent heterochromatic region (qh) of chromosomes 1, 16, and sometimes 9 in mitogen-stimulated lymphocytes. These rearrangement-prone regions show DNA hypomethylation in all examined ICF cell populations. The method includes detecting CAP body formation, as a biomarker, in a cell present in a sample from a mammal (e.g., a human). In an embodiment, CAP body formation is due to demethylation of Sat II DNA on 1q12. In another embodiment, detection of CAP body formation in a cell of the mammal indicates that the mammal has ICF.

In embodiments of the first, second, third, seventh, eighth, and ninth methods, the method further includes detecting, in a cell of the sample, a biomarker selected from one or more of a) an unbalanced distribution of one or more polycomb proteins (resulting in, e.g., an impaired ability to monoubiquitylate histone H2A or an unbalanced distribution of heterchromatic markers), relative to the distribution in a normal cell; b) an unbalanced distribution of a heterochromatic marker (e.g., one or more of monoubiquitylated histone H2A, H3K27me, H3K9me2, HP1, H4K20me, loss of H3K4me, loss of H4Ac, DNA methylation (5-mC), and macroH2A) in the nucleus of a cell in the sample, relative to the distribution in a normal cell (e.g., an increase or decrease in the amount of the heterochromatic marker present in the nucleus, or of a redistribution of the heterochromatic marker into prominent foci that are, e.g., largely absent in normal cells; and c) a mutant BRCA1 protein that exhibits an impaired ability to monoubiquitylate histone H2A, relative to wild-type BRCA1 protein, or a mutant BRCA1 gene that encodes the mutant BRCA1 protein, relative to a normal cell. In an embodiment, the detecting step includes, e.g., detecting the distribution, level, or presence of the biomarker(s).

In embodiments of the first, second, third, and ninth methods, the CAP body includes a satellite II deoxyribonucleic acid (DNA) molecule and/or the CAP body includes a polycomb group protein (e.g., the polycomb group protein is a PRC1 or PRC2 complex protein; in particular, the PRC1 complex protein is selected from BMI-1, RING 1B, Phc1, Phc2, CBX4, CBX8, and RNF2 or the PRC2 complex protein is one or more of SUZ12, EED, RBBP4, JARID2, EZH2, EZH1, and RBBP7) or a protein that interacts with the PRC1 complex (e.g., GLI1, MYC, CDKN2A, and HST2H2AC). In other embodiments, the CAP body is present at the 1q12 or 16q11 DNA locus in the nucleus of cell(s) of the sample.

In an embodiment of the first, second, and third methods, the detection of satellite II RNA is by direct visual analysis of cell(s) by microscopy following binding of a detection reagent (e.g., a labeled nucleic acid or LNA probe) to satellite II RNA in the cell(s) of the sample. In another embodiment, the detection of satellite II RNA includes quantifying the amount present in the nucleus of a cell(s) of the sample or its distribution within the nucleus. In still other embodiments, the satellite II RNA is quantified by digital microfluorimetry. In yet other embodiments, the amount of satellite II RNA detected in a cancer cell is at least 1, 2, 3, 4, 5, 6, 7, 8, 9, or 10 fold higher than in a normal cell, more preferably 15, 20, 25, 30, 35, 40, 45, or 50 fold higher than in a normal cell, and most preferably 60, 70, 80, 90, 100, 110, 120, 130, 140, 150, 160, 170, 180, 190, 200, 250, 300, or 350 fold or more higher than in a normal cell (e.g., about 175 fold higher than in a normal cell). In an embodiment, the prominent aberrant foci of satellite II RNA are a unique “signature” of cancer cells, which can mark even a single cancer cell as distinct from normal, by direct visual analysis or quantitative digital microscopy.

In other embodiments of the first, second, third, fourth, fifth, sixth, seventh, eighth, and ninth methods, the difference in signal (CAP, CAST and UbH2A) between cancer and normal cells can be reduced to two parameters that are clearly visible by eye and/or can be easily quantified by one with skill in the art. They are “distribution” and “intensity.” The distribution of these biomarkers is clearly visibly different for cancer cells and easily differentiates cancer cells from normal cells (e.g., in in vitro, in situ, and ChIP results). The highest intensity signal (pixel intensity by microscopy, and peak height for ChIP) in a cancer nucleus is higher than any signal in a normal cell for these marks and can be quantified (as discussed above).

In other embodiments of the first, second, and third methods, the CAST body includes the satellite II ribonucleic acid (RNA) molecule, e.g., a cytosine methylated satellite II RNA molecule, and/or the CAST body includes proteins containing an RNA binding domain and/or proteins that are involved in RNA metabolism, such as a methyl DNA binding protein (e.g., the methyl DNA binding protein is methyl CpG (cytosine phosphate guanine) binding protein 2 (MeCP2)), a protein known to interact with MeCP2 (e.g., one or more of SIN3A, CDKL5, DNMT1, HDAC1, ATRX, DNMT3B, SMARCA2, DLX5, BDNF, and UBE3A), or a protein known to become sequestered on similar repeat RNA aggregates in microsatellite repeat diseases (e.g., one or more of MBNL 1, 2, and 3, hnRNP H, G, A, and K, proteosome 20Sα, 11Sγ and 11sα subunits, Y12, Y14, 9G8, snRNP Sm antigen, SAM68, SLM 1 and 2, Tra2β, Purα, and CPEB proteins).

In other embodiments of the first, second, and third methods, the CAST body includes an alpha-satellite RNA.

In embodiments of the first, second, third, fourth, fifth, sixth, seventh, eighth, and ninth methods, the method may include detecting the biomarker(s) using a serum screen or detecting one or more of the biomarker(a) (e.g., the satellite II RNA molecule or the UbH2A) using reverse transcriptase polymerase chain reaction (RT-PCR; e.g., quantitate real-time PCR), a microarray, a deep sequencing assay (e.g., a ChIP-Seq assay), or microscopy. The satellite II RNA molecule detection assay may utilize a nucleic acid molecule or a locked-nucleic acid (LNA) oligo as a probe (unbound or bound to a solid support). In other embodiments, the method may involve detecting the Satellite II RNA molecule using a probe having at least 50% (e.g., 55%, 60%, 65%, 70%, 75%, 80%, 85%, 90%, 95%, 97%, 99%, or 100%) sequence identity (preferably 80% or more sequence identity) over at least 20 or more (e.g., 21, 22, 23, 24, 25, 26, 27, 28, 29, or 30 or more) consecutive nucleotides of one or more of SEQ ID NOs: 14 to 28. In an embodiment, the probe is capable of specifically hybridizing under stringent conditions to a nucleic acid molecule having the sequence of one or more of SEQ ID NOs: 14-28. In an embodiment, the detecting step includes, e.g., detecting one or more of the distribution, level, or presence of the biomarker(s) in the nucleus of at least one cell in the sample.

In still other embodiments of the first, second, third, fourth, fifth, sixth, seventh, eighth, and ninth methods, the method may include detecting the biomarker(s) (e.g., detecting one or more of the distribution, level, or presence of the biomarker(s)) using radioimmunoassay (RIA), enzyme-linked immunosorbent assay (ELISA), immunoblotting, immunoprecipitation, or microscopy (e.g., the microscopy is in situ fluorescence microscopy, such as immunofluorescence microscopy, indirect-immunofluorescence, immunocytochemistry, or immunohistochemistry). In another embodiment, the method may include detecting the CAP body using microscopy (e.g., the microscopy is in situ fluorescence microscopy, such as immunofluorescence microscopy, indirect-immunofluorescence, immunocytochemistry, or immunohistochemistry). Immunoprecipitation used in either method may be chromatin immunoprecipitation (e.g., the chromatin immunoprecipitation may include one or more of the following step: digesting the genome of the cell(s) in the sample, contacting an antibody that specifically binds one or more proteins of the CAP body to the digested genome in the sample, separating an antibody/CAP body/chromatin complex that includes DNA from the sample, and/or sequencing the DNA from the antibody/CAP body/chromatin complex (e.g., the presence of a satellite II DNA sequence within the antibody/CAP body/chromatin complex indicates the sample includes the cancer cell(s)). In still other embodiments, the immunoprecipitation used in the method may include one or more of the following steps: digesting the genome of the cell(s) in the sample, contacting a nucleic acid molecule complementary to and specific for a satellite II DNA sequence to the digested genome to form a hybridization complex, separating the hybridization complex from the sample, and/or contacting one or more components of the hybridization complex with an antibody that specifically binds to one or more proteins of the CAP body (e.g., binding of the antibody to one or more of the proteins of said CAP body indicates the sample includes the cancer cell(s)). The methods can also include quantification of the amount of the biomarker(s), e.g., using an automated pathology platform. The quantification may be digital quantification.

In other embodiments of the first, second, third methods, the method may include detecting the satellite II RNA molecule or the alpha-satellite RNA molecule in the sample using a method selected from a microarray, RNA fluorescence in situ hybridization (FISH), northern blot, polymerase chain reaction (PCR), RNA sequencing, and microscopy. In still other embodiments of the first, second, third, and ninth methods, detecting the satellite II DNA molecule in the sample may include a method selected from a microarray, DNA fluorescence in situ hybridization (FISH), Southern blot, polymerase chain reaction (PCR), DNA sequencing, and microscopy. In an embodiment, the detecting step includes, e.g., one or more of detecting the distribution, level, or presence of the biomarker(s).

In yet other embodiments of the first, second, third, fourth, fifth, sixth, seventh, and ninth methods, the biomarker(s) is detected with one or more antibodies (e.g., one or more antibodies to at least one CAP body protein, at least one CAST body protein, or at least one heterochromatic marker (e.g., one or more of histone H2A, H3K27me, H3K9me2, HP1, H4K20me, loss of H3K4me, loss of H4Ac, DNA methylation (5-mC), and macroH2A)). In other embodiments, the methods include detection of at least two proteins (e.g., three, four, five or more proteins) of the CAP or CAST bodies using two antibodies (or a number of antibodies commensurate with the number of proteins to be detected), each of which is capable of specifically binding to a different CAP or CAST body protein. For example, detection of the CAP or CAST bodies may include the use of a first antibody that is capable of specifically binding to a first protein in the CAP or CAST body, and a second antibody that is capable of specifically binding to a second, different protein in the CAP or CAST body. In particular embodiments, the methods include the use of, e.g., one or more (e.g., two, three, four, five, or more) antibodies that specifically bind one or more of the polycomb group protein(s) of the CAP body, such as the PRC1 or PRC2 complex protein(s) or their associated protein(s) (for example, one or more of BMI-1, RING 1B, Phc1, Phc2, CBX4, CBX8, RNF2, SUZ12, EED, RBBP4, JARID2, EZH2, EZH1, RBBP7, GLI1, MYC, CDKN2A, or HST2H2AC), or one or more (e.g., two, three, four, five, or more) antibodies that specifically bind one or more proteins of the CAST body (for example, one or more of MeCP2, SIN3A, CDKL5, DNMT1, HDAC1, ATRX, DNMT3B, SMARCA2, DLX5, BDNF, UBE3A, MBNL 1, 2, and 3, hnRNP H, G, A, and K, proteosome 20Sα, 11Sγ and 11sα subunits, Y12, Y14, 9G8, snRNP Sm antigen, SAM68, SLM 1 and 2, Tra2β, Purα, or CPEB proteins), or one or more (e.g., two, three, four, five, or more) antibodies that specifically bind histone H2A).

In other embodiments of the first, second, and third methods, the satellite II RNA molecule or the alpha-satellite RNA molecule is detected using a probe (e.g., a probe having a sequence with at least 50% (e.g., 55%, 60%, 65%, 70%, 75%, 80%, 85%, 90%, 95%, 97%, 99%, or 100%) sequence identity (preferably 80% or more sequence identity) to a sequence that is complementary to, and specific for, a Sat II RNA, such as a probe selected from Sat2-24 nt LNA, Sat2-24 nt, Sat2-59 nt, and Sat2-169 bp, or a probe having a sequence with at least 50% (e.g., 55%, 60%, 65%, 70%, 75%, 80%, 85%, 90%, 95%, 97%, 99%, or 100%) sequence identity (preferably 80% or more sequence identity) to a sequence that is complementary to, and specific for, an alpha-satellite RNA, such as HuAlphaSat). In other embodiments, the probe has a sequence with at least 80% sequence identity to the sequence of SEQ ID NOs: 2 to 10, or its complement. In still other embodiments, the probe includes a sequence having at least 50% (e.g., 55%, 60%, 65%, 70%, 75%, 80%, 85%, 90%, 95%, 97%, 99%, or 100%) sequence identity (preferably 80% or more sequence identity) to a sequence of at least 20 consecutive nucleotides (e.g., at least 21, 22, 23, 24, 25, 26, 27, 28, 29, 30, or more, or the entire sequence) set forth in SEQ ID NOs: 14 to 28. In another embodiment, the probe is capable of specifically hybridizing under stringent conditions to a nucleic acid molecule having the sequence of one or more of SEQ ID NOs: 14-28. In yet another embodiment, the probe is an LNA probe. The LNA probe optionally has at least 50% (e.g., 55%, 60%, 65%, 70%, 75%, 80%, 85%, 90%, 95%, 97%, 99%, or 100%) sequence identity to the complement of the target nucleic acid molecule sequence. In other embodiments, hybridization of the probe to the satellite II RNA molecule or the alpha-satellite RNA molecule is detected by microscopy.

In other embodiments of the first, second, third, fourth, fifth, sixth, seventh, eighth, and ninth methods, the sample includes an organ, tissue, cell, bodily fluid (e.g., saliva, serum, plasma, blood, urine, mucus, gastric juices, pancreatic juices, semen, products of lactation or menstruation, tears, or lymph), lavage (e.g., bronchalveolar lavage, a gastric lavage, a peritoneal lavage, a vaginal lavage, a colonic or rectal lavage, an arthroscopic lavage, a ductal lavage, or an ear lavage), skin, hair, or fecal matter from the mammal.

By “sequence identity” or “sequence similarity” is meant that the identity or similarity between two or more amino acid sequences, or two or more nucleotide sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Additional information can be found at the NCBI web site.

BLASTN is used to compare nucleic acid sequences, while BLASTP is used to compare amino acid sequences. To compare two nucleic acid sequences, the options can be set as follows: −i is set to a file containing the first nucleic acid sequence to be compared (such as C:\seq1.txt); −j is set to a file containing the second nucleic acid sequence to be compared (such as C:\seq2.txt); −p is set to blastn; −o is set to any desired file name (such as C:\output.txt); −q is set to −1; −r is set to 2; and all other options are left at their default setting. For example, the following command can be used to generate an output file containing a comparison between two sequences: C:\B12seq c:\seq1.txt −j c:\seq2.txt −p blastn −o c:\output.txt −q −1 −r 2.

To compare two amino acid sequences, the options of B12seq can be set as follows: −i is set to a file containing the first amino acid sequence to be compared (such as C:\seq1.txt); −j is set to a file containing the second amino acid sequence to be compared (such as C:\seq2.txt); −p is set to blastp; −o is set to any desired file name (such as C:\output.txt); and all other options are left at their default setting. For example, the following command can be used to generate an output file containing a comparison between two amino acid sequences: C:\B12seq c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid or nucleotide residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (i.e., 1166=1554*100=75.0). The length value will always be an integer. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. By “specifically binds” is meant the preferential association of a binding moiety (e.g., an antibody or fragment thereof) to a target molecule (e.g., a polycomb group protein of the CAP body, such as a PRC1 or PRC2 complex protein or an associated protein (for example, BMI-1, RING 1B, Phc1, Phc2, CBX4, CBX8, RNF2, SUZ12, EED, RBBP4, JARID2, EZH2, EZH1, RBBP7, GLI1, MYC, CDKN2A, and HST2H2AC), a protein of the CAST body (for example, MeCP2, SIN3A, CDKL5, DNMT1, HDAC1, ATRX, DNMT3B, SMARCA2, DLX5, BDNF, UBE3A, MBNL 1, 2, and 3, hnRNP H, G, A, and K, proteosome 20Sα, 11Sγ and 11sα subunits, Y12, Y14, 9G8, snRNP Sm antigen, SAM68, SLM 1 and 2, Tra2β, Purα, and CPEB protein), or histone H2A) in a sample (e.g., a biological sample) or in vivo or ex vivo. It is recognized that a certain degree of non-specific interaction may occur between a binding moiety and a non-target molecule. Nevertheless, specific binding may be distinguished as mediated through specific recognition of the target molecule. Specific binding results in a stronger association between the binding moiety (e.g., an antibody or fragment thereof) and, e.g., an antigen (e.g., a CAP body protein, a CAST body protein, or histone H2A) than between the binding moiety and, e.g., a non-target molecule (e.g., a non-CAP body protein, a non-CAST body protein, or non-histone H2A protein). For example, an antibody specifically binds if it has, e.g., at least 2-fold greater affinity (e.g., 2, 3, 4, 5, 6, 7, 8, 9, 10, 10²-, 10³-, 10⁴-, 10⁵-, 10⁶-, 10⁷-, 10⁸-, 10⁹-, or 10¹⁰-fold greater affinity) to an epitope of a CAP body protein, a CAST body protein, or histone H2A than to polypeptides other than a CAP body protein, a CAST body protein, or histone H2A.

By “stringent conditions” is meant conditions under which an oligonucleotide probe will selectively or specifically hybridize to its target sequence (e.g., a satellite II RNA or DNA sequence), typically in a complex mixture of nucleic acids, but to no other sequences. Stringent conditions are sequence-dependent and length-dependent. Generally, stringent conditions are selected to be about 5° C. to about 25° C. lower than the thermal melting point (Tₘ) for the specific sequence at a defined ionic strength pH. Stringent conditions may also include destabilizing agents, such as formamide. For selective or specific hybridization, a positive signal is at least two times background, preferably 10 times background hybridization. Exemplary stringent conditions include: 50% formamide, 4×SSC, and 1% SDS, incubating at 42° C.; and 4×SSC, 1% SDS, incubating at 65° C., with wash in 0.2×SSC, and 0.1% SDS at 65° C. Hybridization techniques are generally described in Nucleic Acid Hybridization, A Practical Approach (eds. B. D. Hames and S. J. Higgins, IRL Press, 1985); Tijssen, “Overview of principles of hybridization and the strategy of nucleic acid assays” in Laboratory Techniques in Biochemistry and Molecular Biology: Hybridization with Nucleic Probes (ed. P. C. van der Vliet, Elsevier Science Publishers B.V., 1993); PCR Protocols, A Guide to Methods and Applications (eds. M. A. Innis et al., Academic Press, Inc., New York, 1990); Gall and Pardue, Proc. Natl. Acad. Sci., USA 63:378-383, 1969; and John et al., Nature 223:582-587, 1969.

Other features and advantages of the invention will be apparent from the following Detailed Description, the drawings, and the claims.

### BRIEF DESCRIPTION OF THE DRAWINGS

FIGS. 1A-1J. Cot-1 RNA exhibits bright foci in cancer cells that are revealed as Sat II. FIG. 1A is a fluorescent photomicrograph showing Cot-1 RNA staining with DAPI in HT1080 fibrosarcoma cells. Scale bar is 10 um (images A-D at same scale). FIG. 1B is a fluorescent photomicrograph showing Cot-1 RNA staining with DAPI in normal fibroblasts. Normal fibroblasts show only the normal nucleoplasmic Cot-1 RNA signal. FIG. 1C is a fluorescent photomicrograph showing staining of histone mRNA transcription foci with DAPI. The histone mRNA foci are small relative to Cot-1 RNA foci. FIG. 1D is a fluorescent photomicrograph showing DAPI staining of Cot-1 RNA foci compared to the large XIST RNA territory. FIG. 1E is a table showing that eight of nine cancer lines are positive for Cot-1 RNA foci, while none of the normal lines (asterisk) exhibited them. FIGS. 1F and 1G are fluorescent photomicrographs showing that Cot-1 RNA foci are not due to over expression of SINES (Alu) (FIG. 1F) or LINES (L 1) (FIG. 1G). Scale bar is 10 um (images F-G same scale). FIG. 1H is a fluorescent photomicrograph showing that Sat II RNA most often overlaps the Cot-1 RNA foci in cancer cells. Scale bar 10 um. Photomicrographs showing only the Cot-1 RNA foci and the Sat II RNA foci are shown below FIG. 1H. FIG. 1I is a fluorescent photomicrograph showing that HT1080 was only one of two cancer lines examined that show alpha-satellite in the Cot-1 RNA foci. FIG. 1J is a linescan of the cell in FIG. 1I quantifying the alpha-satellite RNA in different Cot-1 RNA foci.

FIGS. 2A-2F. Digital microfluorimetry quantifies the dramatic difference in Sat II RNA signal in cancer and normal cells. FIGS. 2A-2C are fluorescent photomicrographs showing that cancer cells (FIGS. 2A and 2B) contain aberrant Sat II foci, while normal fibroblasts (FIG. 2C) do not. DNA is stained with DAPI (blue). All images are of equal exposure and magnification (bar is 10 um). FIG. 2D is a linescan through the nucleus of three cancer cells (HCC-1937, MCF-7 & PC3), and two normal cells (Tig-1 & WS1) demonstrating the size (peak width), intensity (peak height) and number (# of peaks) of Sat II RNA foci in these cells. FIG. 2E is a graph in which the single brightest intensity pixel in each of 50 cells for two cell lines (IMR90/normal & U2OS/cancer) are plotted in relation to the threshold for each line (threshold=3× average minimum pixel intensity). Most normal cells fall below the threshold, while few cancer cells do. FIG. 2F is a graph showing the total Sat II RNA signal per cell. The total Sat II RNA signals above threshold in 10 cells for cancer (U2OS) and normal (IMR90) lines (including very faint foci in six of the ten normal cells) were quantified (intensity and area) and the average plotted.

FIGS. 3A-3J. BMI-1 localizes in large aberrant foci, forming cancer-associated PcG (CAP) bodies. FIGS. 3A and 3B are fluorescent photomicrographs showing cancer cells with large accumulations of BMI-1 protein in CAP bodies. Normal fibroblasts (FIGS. 3C and 3-D exhibit only a lower level nucleoplasmic punctate signal. FIG. 3E is a graph showing that seven of eight cancer cell lines show a high percentage of cells with CAP bodies while non-cancer lines do not. FIGS. 3F and 3G are photomicrographs showing that fibroblasts (FIG. 3F) exhibit low levels of the nucleoplasmic BMI-1, and telomerase immortalized RPE cells had slightly higher levels (FIG. 3G). FIG. 3H is a photomicrograph showing that U2OS cancer cells exhibit very high concentrations of BMI-1 in CAP bodies, but very low levels in the nucleoplasm. FIGS. 3F-3H are in the same scale. FIGS. 3I and 3J are linescans measuring different nucleoplasmic BMI signals in normal cells (FIG. 3I) and CAP bodies versus low nucleoplasmic levels in cancer cells (FIG. 3J).

FIGS. 4A-4K. Sat II RNA is expressed from smaller Sat II DNA loci which are not associated with BMI-1. FIG. 4A is a fluorescent photomicrograph showing that Sat 2-59 oligo labels predominantly Chr1 and Chr16, and a few other loci at low levels (e.g., Chr 2 and 15 in insert), while the Sat 2-24 LNA oligo labels considerably more loci, including the Sat III locus on Chr9 under low stringency. Inserts show separated color channels. FIGS. 4B-4D are photomicrographs showing that Sat II DNA loci labeled using the puc 1.77 kb (Chr1q12) probe are consistently associated with BMI-1 bodies in cancer cells. Note: this probe also labels very small loci on other chromosomes that accumulate BMI-1 as well (arrows). FIGS. 4E-4K are photomicrographs showing that Sat II RNA is expressed from the smaller Sat II DNA sites, and not from the larger ones. FIGS. 4H-4J show that Sat II RNA slightly overlaps or accumulates adjacent to (FIGS. 4E-4G) the small DNA loci. Inserts are close-ups of selected regions. In FIGS. 4G and 4J, DNA is enhanced to reveal faint signals. FIG. 4K is a linescan of U2OS nucleus showing that Sat II RNA often forms beside the DNA loci and the large DNA signals do not express RNA.

FIGS. 5A-5F. Large aberrant MeCP2 “CAST bodies” are also seen in cancer cells, and associate with Sat II RNA rather than Sat II DNA. FIG. 5A is a fluorescent photomicrograph showing that Sat II RNA foci are not associated with BMI-1 CAP bodies. FIG. 5B is a fluorescent photomicrograph showing that MeCP2 accumulates in large foci completely coincident with Sat II RNA foci in U2OS cancer cells. Inserts are separated channels of two foci from image. FIG. 5C is a linescan across nucleus in image B showing almost complete coincidence of MeCP2 and Sat II RNA distribution. FIG. 5D is a fluorescent photomicrograph showing that MeCP2 and Sat II foci are also coincident in other cancer lines like PC3. Inserts are separated channels of left cell. FIG. 5E is a fluorescent photomicrograph showing that Sat II RNA foci release from mitotic nuclei into the cytoplasm, and FIG. 5F is a fluorescent photomicrograph showing that Sat II RNA foci are still associated with MeCP2, further indicating that the protein is with RNA not DNA. Inserts in FIG. 5F show separated channels (arrow).

FIGS. 6A-6E. Pharmacologically induced DNA hypomethylation in normal nuclei rapidly induces formation of CAP bodies on 1q12 and subsequent RNA expression from other Sat II loci. FIGS. 6A and 6B are photomicrographs showing Normal Tig-1 fibroblasts, 24 hours after treatment with 5-aza-2′deoxycytidine, exhibit aggregations of BMI-1 into large foci resembling CAP bodies. FIG. 6C is a photomicrograph showing that longer treatment (8 days total) results in aberrant expression of Sat II RNA from other loci that are not associated with BMI-1 bodies. FIG. 6D is a photomicrograph showing that, as seen for CAP bodies in cancer cells, BMI-1 bodies formed after 24 hours of treatment in Tig-1 fibroblasts (treated for 24 hours) are localized specifically on 1q12 Sat II DNA. FIG. 6E is a schematic showing the treatment protocol that produced the results shown in FIGS. 6A-6D.

FIGS. 7A-7L. Sat II RNA foci, CAP bodies and CAST bodies are also seen in human solid tumors. FIGS. 7A and 7B are fluorescent photomicrographs showing that Sat II RNA foci are prominent in clustered cells within an ovarian tumor (#2081T) (FIG. 7A) and in most cells of a breast tumor (#2334T) (FIG. 7B) along with BMI-1 CAP bodies. FIG. 7C is a photograph showing an H&E stained section of breast tumor #2334T. FIGS. 7D and 7E are photographs showing that Sat II RNA foci are even visible at the lower magnifications used by pathologists. FIG. 7D shows DAPI staining of DNA only, while FIG. 7E shows DAPI staining plus RNA signal. FIG. 7F is a close-up photograph of a selected region from FIG. 7E. FIGS. 7G and 7H are duplicate photographs showing that BMI-1 protein is highly concentrated in CAP bodies in a kidney tumor cell while the nearby cell lacking a CAP body still contains high nucleoplasmic levels. The line in FIG. 7G is the linescan path. FIG. 7I is a linescan through both cells showing the measurement of high levels of BMI in the CAP body and low nucleoplasmic levels relative to the neighboring cell. FIGS. 7J and 7K are photographs showing that large MeCP2 bodies can also be seen in breast tumor tissues in vivo (FIG. 7J), unlike the fine punctate distribution in matched normal tissue (FIG. 7K). FIG. 7L is a photograph showing that MeCP2 bodies overlap with Sat II RNA foci in breast tumor.

FIG. 8 is a model showing specific Sat II DNA loci and abnormally expressed Sat II RNA underlie formation of aberrant nuclear compartmentalization of epigenetic factors into cancer-associated nuclear bodies, linked to DNA hypomethylation at 1q12. In many cancers, in vitro and in vivo, Sat II RNA is grossly over-expressed and forms prominent nuclear foci. In the same nuclei PRC1 polycomb proteins, BMI-1 and Ring1B, aggregate abnormally to form prominent bodies on a subset of Sat II loci, primarily the largest (˜6 Mb) Sat II locus at 1q12, enriched for a distinct sub-type of Sat II sequences. These prominent bodies of PcG proteins on 1q12 (and 16q11) are not normal nuclear structures, and thus are termed Cancer-Associated Polycomb “CAP” bodies. While Sat II loci with CAP bodies remain silent, Sat II RNA foci emanate from smaller Sat II loci in the broader nuclear compartment thus depleted of BMI-1. In addition, MeCP2 then redistributes to accumulate on the abnormal Sat II RNA foci, forming Cancer-associated Satellite Transcript (“CAST”) bodies. The prominent 1q12 associated PcG bodies are rapidly induced in normal cells by treatment with a DNA demethylating agent.

FIGS. 9A-9H. FIGS. 9A-9D are fluorescent photomicrographs showing that Cot-1 RNA signals in a number of different cancer cell lines, including Hela (FIG. 9A), MCF-7 (FIG. 9B), HCC1937 (FIG. 9C), and SUM-149PT (FIG. 9D), show bright repeat RNA foci. FIGS. 9E and 9F are duplicate photomicrographs showing that Sat II RNA and Poly-A RNA in U2OS cells indicate that these RNA foci are not polyadenylated since they reside in a “hole” in the Poly-A signal (see the arrow in FIG. 9F). FIGS. 9G and 9H are fluorescent photomicrographs of DAPI-stained U2OS cells showing that Sat II RNA foci are removed with RNAse treatment (FIG. 9G shows control cells, while FIG. 9H shows RNase treated cells). Similar levels of nucleoplasmic signals are present in all cell lines but this is less apparent in images where the focal RNA is extremely bright, as is the case for all lines except Hela.

FIGS. 10A-10F. FIG. 10A is a photomicrograph of DAPI-stained Tig-1 cells showing that alpha-satellite RNA foci were unexpectedly visible, clearly and consistently, in all normal cell samples examined. FIG. 10B is a photomicrograph of DAPI-stained HSMM myotube cells showing that alpha-satellite RNA foci were even apparent in non-cycling cells like these G0 differentiated myotube cells (as well as the cycling myoblasts). These alpha-satellite signals were confirmed as RNA by their removal by RNAse, as shown in FIGS. 10C (control) and 10D (RNase treated) as well as by their absence on centromeres of mitotic chromosomes. FIG. 10E is a photomicrograph of DAPI-stained HT1080 cells showing that alpha-satellite RNA foci are sometimes seen in the cytoplasm of mitotic cells where they have been released from the nucleus during mitosis. FIG. 10F is a graph showing that normal-cell alpha-sat RNA foci were not as large and robust as the Cot-1 RNA or Sat II RNA foci in cancer cell nuclei, but nonetheless 2-20 small RNA foci were readily apparent, without image processing in 65% to 97% of the normal cell populations.

FIGS. 11A-11F. FIG. 11A is a photomicrograph of DAPI-stained chromosomes from US02 cells. The Sat 2-59 oligo and the PCR generated Sat 2-160 probe both label the Sat II loci at Chr1q12 and Chr 16, as well as a small Sat II loci on a few other chromosomes (Chrs. 2, 10, 15). FIG. 11B is a photomicrograph of DAPI-stained US02 cells showing that the Sat II RNA signal detected by the Sat 2-24 LNA oligo is not significantly diminished when hybridized at higher stringency (40% formamide). FIGS. 11C-11F are photomicrograph of showing that the lqt 2 Sat II loci and the tiny sat 2 DNA loci labeled with the Sat 2-160 PCR probe are associated with BMI-1 CAP bodies (separated channels to the right). The Sat II DNA image is enhanced in FIG. 11F to show the dimer Sat II DNA loci associated with BMI-1.

FIGS. 12A-12D. FIGS. 12A and 12 are photomicrograph of stained US02 cells showing. Because Sat 2 sequences are degenerate versions of the more conserved 5 bp Sat 3 sequence and often contain these sequences, the Sat 3 oligo, under low stringency, could detect the same Sat II RNA foci as the Sat 2-24 LNA oligo. Only rarely, in unusual U2OS cells (<1%), were there one or two RNA foci that contained only Sat 3 sequences (top right in FIG. 12A). FIG. 12C is a photomicrograph of showing that the Sat 3 oligo hybridized to DNA predominantly on the Sat III locus on Chr 9 in US02 cells. FIG. 12D a photomicrograph of showing that, after enhancement of the image of FIG. 12C, very dim signals can be seen on Sat II loci on other chromosomes, including Chr 1.

FIGS. 13A-13K. FIGS. 13A-C are photomicrographs of US02 cells showing that the PcG protein EZH2 (from the PRC2 complex) is usually not found in the same CAP bodies as BMI-1 in U2OS cancer cells, as previously reported. FIGS. 13D-13F are photomicrographs showing that in the PC3 cancer cell line, EZH2 is more concentrated in BMI-bodies but that nucleoplasmic levels of EZH2 are not as depleted as for BMI-1. FIGS. 13G-13I are photomicrographs showing that RING1B is also found in CAP bodies in U2OS, with very low nucleoplasmic levels, consistent with other studies showing colocalization with BMI-1 in bodies lacking EZH2. FIG. 13J is a photomicrographs showing staining of Phc-1, which is also a member of the PRC1 complex, in CAP bodies with BMI-1 in PC3 cells. FIG. 13K is a photomicrograph showing that Sat II RNA is not associated with the perinucleolar compartment (identified using the PTBP1 protein), despite Sat 2 RNA foci often being peripheral to the nucleolus.

FIGS. 14A-14E. FIGS. 14A-14C are photographs showing larger versions of the low-mag images of breast tumor #23341 sections showing H & E staining (FIG. 14A), Sat II RNA (FIG. 14B), and the DNA staining of the same image (FIG. 14C). FIG. 14D is a photograph showing that despite high cytoplasmic autofluorescence, ascites samples exhibited both high levels (++) of cells with Sat II RNA foci, as well as lower levels (+). FIG. 14E is a table showing the detection of Sat II RNA foci in five of nine samples screened. Three of the four negative samples that were screened were benign. All samples were screened blind.

FIGS. 15A-15C. FIG. 15A is a photomicrograph showing that MECP2 or “CAST” bodies are strikingly apparent in the breast tumor sections even at low magnification. DNA (FIG. 15B) and MECP2 (FIG. 15C) color channels are separated for a small section of the field of FIG. 15A.

FIGS. 16A-16B. FIG. 16A is a fluorescent photomicrograph showing that cancer cells in a breast carcinoma contain bright Sat II RNA foci (red) while the normal cells surrounding it do not (lower right). BMI-1 is also shown (green). FIG. 16B shows the same photomicrograph of FIG. 16A but without fluorescence.

FIGS. 17A-17B. FIGS. 17A and 17B are photomicrographs showing that Sat II RNA is overexpressed in cancer cells (HCC-1937; FIG. 17A), but not in normal diploid fibroblasts (Tig-1; FIG. 17B).

FIG. 18. FIG. 18 is a photomicrograph showing PcG protein sequestration. One cell (left) shows BMI protein localized into bodies and no nucleoplasmic signal, while the other cell (right) shows only dispersed BMI and no bodies.

FIGS. 19A and 19B. FIGS. 19A and 19B are schematics showing genome-wide UbH2A ChIP-seq results in U2OS osteosarcoma cells (FIG. 19A) compared to Tig-1 normal fibroblasts (FIG. 19B). The results show an imbalanced distribution of ubiquitylated histone H2A (laid down by PRC1 complex) in the U2OS cancer cells relative to the Tig-1 normal fibroblasts.

FIG. 20A-20C. FIG. 20A is a fluorescent photomicrograph showing labeling of BRCA1 in mouse nuclei, which have prominent chromocenters reflecting a defined organization of centric and pericentric heterochromatin. FIG. 20B is a fluorescent photomicrograph showing mouse nuclei labeled for UbH2A. The overlap and association of BRCA1 foci with UbH2A can be striking, particularly in a subset of cells that label with PCNA, a replication marker (see FIG. 20C and three inset images which are magnified from the larger image).

### DETAILED DESCRIPTION OF THE INVENTION

Currently pathologists rely on changes in nuclear morphology to facilitate diagnosis of many cancers, but this is a relatively crude assay. Our discovery is that prominent nuclear accumulations of Sat-II RNA are a common property of cancer cells in vitro, and in vivo, reflecting compromised heterochromatic silencing in cancer cells, and that these RNA accumulations are capable of sequestering large amounts of regulatory proteins, which may further affect the cancer epigenome. Thus, we discovered that the mis-regulation of satellite RNAs is a characteristic “signature” of cancer cells. Our discovery suggests that gross over-expression of certain repeat RNAs is a common and robust manifestation of cancer cells, which differentiates it from normal cells. This usually involves the over-expression of satellite II (Sat II) RNA primarily, but there are also indications that other satellite sequences may be mis-regulated in cancers as well, such as alpha-satellite RNA.

Thus, a first aspect of the invention features the use of Sat II RNA as a biomarker for diagnosing cancer (e.g., metastatic cancer) in a mammal (e.g., a human).

The abundant Sat II repeat transcripts seen in cancer cells are not just inert by-products of epigenetic dysregulation, but can contribute to further imbalance of the epigenome. We find that Sat II RNA foci are associated with large amounts of the methyl-DNA binding protein, MeCP2 in cancer cells. This suggestion that abnormal conglomerations of repeat RNAs could “compartmentalize” nuclear factors, and thereby potentially impact expression of other genes, has strong precedence based on “toxic repeat RNAs” in certain triplet repeat diseases. Nuclear accumulations of mRNA containing CUG repeats sequester MBNL1, an alternative splicing factor, causing inappropriate splicing patterns that generate the Myotonic Dystrophy (DM1) phenotype. It is also notable that MeCP2, like MBNL1, is implicated in alternative splicing, and is also frequently altered in cancer. We reason that the abundant Sat II RNAs in cancer nuclei may have as much or more capacity to “soak up” regulatory factors as do the repeat containing RNA in DM1.

We conducted a broad survey of Cot-1 repeat RNA expression and distribution in human interphase nuclei. While competition with unlabelled Cot-1 DNA (repetitive genomic fraction) is often used to suppress hybridization to repeats, instead we labeled human Cot-1 DNA as a probe to examine the distribution of transcripts from the repeat genome by RNA FISH. In 2002 we were the first to publish that hybridization to Cot-1 RNA provides a convenient assay to evaluate chromosome inactivation within nuclei, and in 2007 used it in a manuscript to reveal breakdown of the peripheral heterochromatic compartment in cancer cells. However, the discovery that repeat RNAs were aberrantly expressed in cancer began when we initially observed large localized foci of Cot-1 RNA in several cancer cell lines in 2002, which were largely absent in normal cells. Because Cot-1 DNA is a complex probe containing several major classes of repeats, in 2005 we began to use probes to specific repeats to better define the content of these large Cot-1 RNA foci.

We found that interspersed repeats like long interspersed elements (LINEs) or short interspersed elements (SINEs) were not responsible for the large repeat RNA foci, and alpha-satellite accounted for some foci in only a few lines, but the majority of Cot-1 RNA foci in most cancer cell lines are comprised primarily of Sat II RNAs. A survey of cell lines shows that several cancer lines, representing different types of cancers (see Tables 2-6 below), exhibit prominent foci of Sat II RNA in the vast majority (70-100%) of cells, while none of the normal lines did. Prominent foci of alpha-sat RNA were also observed in some of the cancer tissues (see, e.g., Tables 3 and 4 below), but not in matched normal tissue. Evidence also suggests this is single-stranded and non-polyadenylated RNA, and shows some expression from the “reverse” strand. Similarly, although RNA preservation was often compromised in human primary samples, we also find large Sat II RNA foci in 5 of 6 malignant human effusions and 0 of 3 benign effusions, and in 5 of 6 solid human tumor samples (from breast, kidney, ovary and pancreas) while none of 3 matched normal samples nor the normal cell types present in the tumor samples had them. Several cancer tissues tested also exhibited prominent foci of Sat II DNA and its associated proteins (see Table 4 below). Thus, we find that gross over expression of satellite RNAs, and the presence bodies associated with Sat II DNA, is a common and previously unrecognized “hallmark” of many cancers.

The Sat II RNA over-expression itself provides a potentially useful biomarker, and indicator of heterochromatic instability, but these repeat RNAs would clearly have additional significance if they actually impact the cell and/or epigenome in some way, like the “toxic repeat RNAs” in certain triplet repeat expansion diseases (see above). We find that the DNA methyl binding protein, MeCP2, which plays a role in mRNA processing and splice site recognition and shows altered expression in cancer, sharply accumulates in several bright nuclear foci in cancer cells, distinct from the more dispersed and punctuate distribution in normal cells. Co-staining showed that MeCP2 foci do not overlap the Sat II DNA, but rather strictly co-localize with Sat II RNA. In most cells every Sat II RNA focus coincides precisely with an MeCP2 focus both in vitro and in vivo. The MeCP2 foci in primary tumor samples are particularly striking. We find many cells exhibit a pattern of one or a few large, round bright MeCP2 “bodies”, often contrasting with a much darker nucleoplasm, while matched normal tissue showed a higher nucleoplasmic stain with a somewhat variable punctuate pattern, but not large bodies against a dark nucleoplasm. Thus, we refer to this dramatic accumulation of MeCP2 at just a few sites as “cancer-associated satellite transcript” (CAST) bodies, and further corroborates the results suggesting that MeCP2 becomes sequestered with Sat II repeat RNAs in cancer lines. Thus, the aberrant accumulations of Sat II repeat RNAs are not without impact on epigenetic factors in the cell, and MeCP2 “CAST” bodies are another potential biomarker that reflects a highly abnormal cancer epigenome.

Thus, a second aspect of the invention features the use of CAST bodies as a biomarker for diagnosing cancer (e.g., metastatic cancer) in a mammal (e.g., a human).

The presence of satellite RNA and MeCP2 foci provide a readout of cancer cell epigenetics, and may provide robust biomarkers for cancer in general with potential diagnostic value. An important challenge in cancer biology is to identify specific, readily assayed changes that occur in neoplastic progression, which may be common to many cancers, specific to particular types, or indicators of progression level (grade). Knowledge of these changes and how to detect them will be vital for surveillance, recognition and proper classification of different cancers and for designing/evaluating therapeutic interventions. A biomarker could be a cellular, genetic or epigenetic change, such as p53 mutations common in many cancers or a marker such as CYP2W1 that is highly expressed in colorectal tumors. While biomarker discovery is an active area of research, we believe the use of “repeat RNA signatures” or MeCP2 “CAST” bodies as a biomarker for cancer would provide further information on the cancer biology and its aberrant epigenome.

Our studies also show that in cancer nuclei, but not normal nuclei, aberrant aggregations of certain PcG proteins are common (in vitro and in vivo), and form on specific Sat II DNA domains, possibly due to changes in their DNA methylation status. We refer to these aggregations as “cancer associated PcG” bodies (CAP bodies). A third aspect of the invention features the use of CAP bodies as a biomarker for diagnosing cancer. Our discovery provides the first evidence that changes in global methylation (a common hallmark of cancer) particularly at satellite repeats can trigger the dramatic redistribution of epigenetic factors in these cells. The sequestering of these important regulatory factors away from the remaining nucleoplasm is important, and could play a role in the activation of other previously silent genomic loci, like oncogenes or the pericentric satellites (Satellite II) (see above).

Our discovery finds that repeats in the genome (DNA and RNA) organize the distribution of important epigenetic regulators in the nucleus and this goes awry in cancer. We demonstrate that a common feature of cancer nuclei, in vitro and in vivo, is a grossly abnormal nuclear compartmentalization of master epigenetic regulators controlled by changes in methylation of satellite repeats. The hypermethylation and silencing of tumor suppressor genes is a critical mechanistic event in cancer which paradoxically often co-occurs with global hypomethylation, for reasons that are not at all understood. The grossly imbalanced nuclear distribution of master regulatory factors and their link to global demethylating events shown here provides a new way to think about what generates this epigenetic imbalance. In addition to this significance for understanding cancer epigenetics and human satellites, the cancer-specific Sat II RNA and MeCP2 “CAST” bodies (see above) as well as these important related PcG “CAP” bodies, provide new candidate cancer biomarkers, that offer a readout of the “heterochromatic instability” in cancer cells.

Thus, a third aspect of the invention features the use of CAP bodies as a biomarker for diagnosing cancer (e.g., metastatic cancer) in a mammal (e.g., a human).

The invention also features a method for identifying an agent for the treatment of a cancer in a mammal by contacting a cancer cell having a biomarker selected from a cancer-associated polycomb group (CAP) body, a cancer-associated satellite transcript (CAST) body, and a satellite II RNA molecule with a test agent and determining whether the test agent reduces the level of the biomarker by detecting a reduction in the formation of the CAP body or CAST body, or a reduction in expression of the satellite II RNA molecule, in the cancer cell, wherein a reduction in the level of the biomarker in the cancer cell relative to the level of the biomarker in a cancer cell not contacted with the test agent, indicates that the test agent is suitable for the treatment of the cancer.

At minimum, we believe the unusual foci (Sat II RNA and CAST and CAP bodies) that we detect in cancer cells are large and bright enough to provide a useful diagnostic adjunct to the pathologist. The methods of the invention can be used alone or can be used in conjuction with other assays, e.g., cytological assays, for detecting cancer in a subject. Sat II RNA is particularly attractive as a biomarker because it is essentially negative in normal cells, making this a sensitive assay that would also be amenable to extraction-based methodologies like RNA microarrays or a deep-sequencing approach, and possibly through serum screens as well. We also find that these bright foci lend themselves easily to simple digital quantification, which can be utilized in automated pathology platforms currently being designed by many companies (e.g. GE Global). For example, quantifying the single brightest pixel per nucleus clearly differentiated normal cells from cancer cells, and suggested a 175 fold difference between normal and cancer cells (see Example I below). This direct visualization of epigenetic regulatory factors within the nucleus of single cells can overcome the limitations of extraction based methodologies that may be “contaminated” by normal cells in the tumor sample. In addition, the methods described herein can be used to diagnose cancer by detecting aberrant localization of at least one (or two or more) protein(s) (e.g., one or more of MeCP2, SIN3A, CDKL5, DNMT1, HDAC1, ATRX, DNMT3B, SMARCA2, DLX5, BDNF, UBE3A, MBNL 1, 2, and 3, hnRNP H, G, A, and K, proteosome 20Sα, 11Sγ and 11sαsubunits, Y12, Y14, 9G8, snRNP Sm antigen, SAM68, SLM 1 and 2, Tra2β, Purα, or CPEB proteins in CAST bodies or one or more of BMI-1, RING 1B, Phc1, Phc2, CBX4, CBX8, RNF2, SUZ12, EED, RBBP4, JARID2, EZH2, EZH1, RBBP7, GLI1, MYC, CDKN2A, or HST2H2AC in CAP bodies) that may not exhibit altered expression in the cancer cell (e.g., the protein levels of the biomarkers in the cancer cell may remain normal relative to a normal, non-cancer cell, but the distribution of the biomarkers across the nucleus in the cancer cell is not “normal” relative to a non-cancer cell). The presence of aberrant accumulations and mis-compartmentalization of key regulatory components of the nucleus in cancer cells provides a robust assay for gross epigenetic mis-regulation in cancer cells and facilitates the evaluation of the tumor or therapy.

These new cancer properties (Sat II RNA and CAST and CAP bodies) are potential “red flags” for cancers in which failed maintenance of chromatin regulation is prominent. Such epigenetic biomarkers are particularly relevant in light of current new chemotherapeutics being tested that target histone modifications or DNA methylation of tumor suppressor genes, but which will likely have unintended consequences on pericentric satellite heterochromatin. Cytopathological changes in nuclear morphology, particularly heterochromatin patterns, are important diagnostic indicators of many cancers, however the distinctions can be subtle and difficult to accurately identify. Since excised tumors often contain just a sub-set of tumor cells mixed with normal, extraction-based assays will dilute the mark present in a small fraction of cells, and, in addition, do not allow direct correlation with the specific diagnostic structural changes upon which the pathologist relies. Thus, an advantage of the biomarkers and approach shown here is that it retains important cytopathology by overlaying these epigenetic hallmarks with cancer morphology at the single cell level, and highlights that epigenomic changes will be more fully understood if the cancer genome is considered as a complex three dimensional entity within a highly subcompartmentalized nuclear structure.

We initially observed the mis-regulation of heterochromatic satellite repeats in cancer cell lines and observed that prominent nuclear accumulations of Sat II RNA were common in many cancer samples in vitro and in vivo, and largely absent in normal cells. Thus, cancer cells show highly aberrant expression of a very abundant satellite repeat which reflects compromised heterochromatic silencing in cancer cells.

To understand why these satellites were being aberrantly expressed in cancer, we examined the proteins known to regulate satellite heterochromatin, the repressive Polycomb Group (PcG) proteins. The PRC1 complex proteins, BMI-1, RING 1B and Phc-1, were of particular interest since these were reported to form Polycomb bodies (PcG bodies) and localize to Sat II DNA domains, particularly tile very large (6 Mb) Sat II block at 1q12, which is commonly hypomethylated in cancer. Although PcG bodies are described as normal nuclear structures we see a dramatic difference between cancer and normal cells. We observed that the PcG proteins are found in a few very prominent nuclear bodies in most cells (70-100%) in 7 of 8 cancer lines, and 4 breast cancer samples, while non-neoplastic cell lines and match normal tissue samples have a more uniform granular or particulate distribution throughout the nucleoplasm. Digital quantification of the high contrast ratio between the PcG bodies versus the nucleoplasm in cancer cells (and normal cells) makes the point that this is a markedly different distribution in cancer, not just higher overall levels. And even if the overall level of the protein is higher in the cancer cell, BMI-1 piles up sharply at a few sites, while the nucleoplasm (where most chromatin resides), has lower levels. Thus, some regions of the cancer nucleus have abundant access to repressive factors, while other regions do not.

We also find that these large aberrant PcG bodies are the same “PcG bodies” that had been previously reported to localize to the large Sat II block on 1q12 (studied in HT1080 cells, a fibrosarcoma cell line). They clearly and consistently (˜100%) co-localize with the 1q12 DNA locus in cancer cells, suggesting a direct relationship between these nuclear elements. Thus, these prominent PcG accumulations which exhibit a high contrast ratio with the nucleoplasm and preferentially “cap” the Sat II locus at 1q12, are a hallmark of cancer cells, and are not a normal nuclear structure. To avoid confusion with the smaller, more numerous and widely dispersed particulate PcG foci in normal human cells, often referred to as “PcG bodies” we refer to the less numerous and larger conglomerations of PcG proteins at 1q12 in cancer cells as “CAP” bodies, for “cancer associated PcG” bodies. Importantly, Sat II DNA loci in other regions of the same nucleus, which contain significantly less PcG proteins than 1q12, are where the aberrant Sat II RNA expression is occurring. This suggests that the mis-compartmentalization of the repressive PRC1 complex from the rest of the nucleus may result in abnormal expression in some areas (e.g. Sat II expression and possibly oncogenes) and abnormal repression in others (possibly at tumor suppressor genes).

The large (6 Mb) Sat II domain on 1q12 is also commonly found hypomethylated in many cancers, and has been reported to be the region most sensitive to changes in methylation. 5-aza-2′-deoxycytidine is a pharmacologic inhibitor of DNA methylation in clinical trials as a chemotherapeutic agent for certain cancers and has also been shown to effectively demethylate Sat II on Chromosome 1. We find that when normal human fibroblasts are treated with this chemotherapeutic agent to hypomethylate 1q12, the PcG proteins of the PRC1 complex re-distribute into large accumulations at 1q12 similar to the CAP bodies seen in cancer cells. Prolonged treatment with this drug (8 days) eventually results in the aberrant expression of Sat II RNA in these normal cells similar to that seen in cancer. This suggests that the abundant satellite repeats have enormous capacity to “soak-up” large quantities of regulatory proteins if the conditions are right (e.g. global demethylation especially at 1q12), resulting in abnormal repression in certain regions of the nucleus, while other regions are abnormally de—repressed.

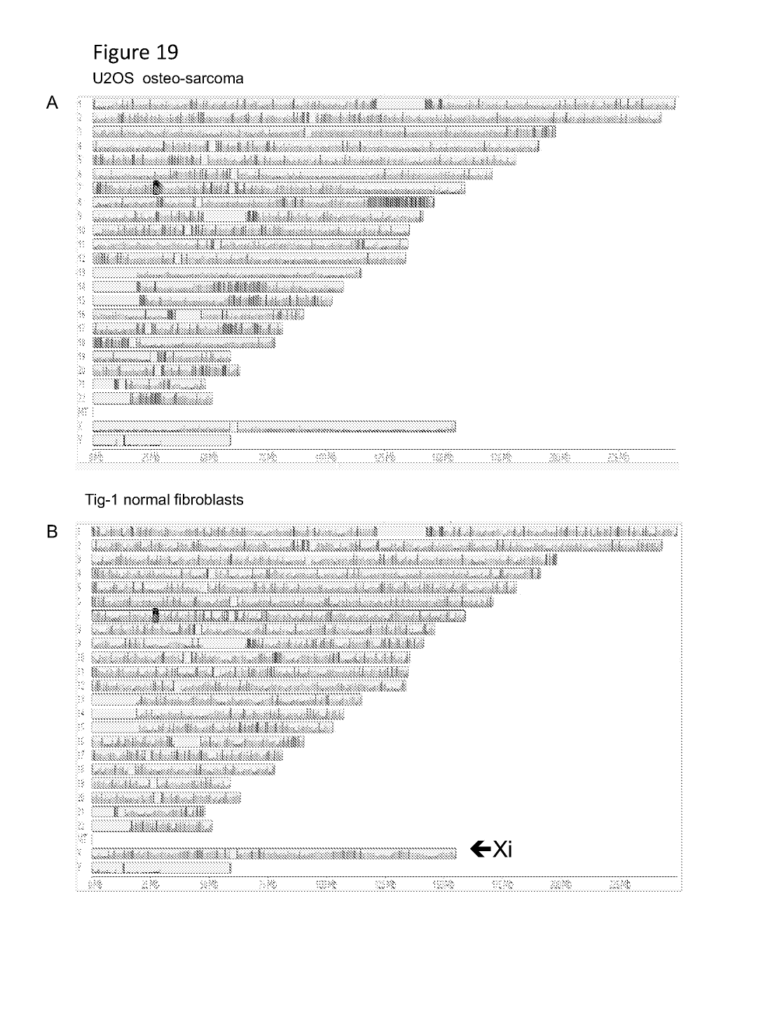

In addition to the use of CAST bodies and their associated Sat II RNA foci or CAP bodies and their associated Sat II DNA foci as biomarkers for the detection of many cancers (see Tables 3 and 4), we have also discovered that cancers can be detected by assaying the unbalanced distribution of heterochromatic markers (e.g., one or more of ubiquitylated histone H2A, H3K27me, H3K9me2, HP1, H4K20me, loss of H3K4me, loss of H4Ac, DNA methylation (5-mC), and macroH2A) in the nucleus of a cell. Our molecular cytology indicates that the cancer nuclear genome has imbalanced (less homogenous) distribution of chromatin regulatory factors due to demethylation of Sat II on 1q12 and its subsequent recruitment of chromatin regulators. Screening for this “unbalanced epigenome” can be done as described above (e.g., by assaying for the presence of cancer associated bodies) or by using a whole genome ChIP-Seq approach (using, e.g., the repressive mark ubiquitin H2A). As shown in FIGS. 19A and 19B, a genome-wide UbH2A ChIP-seq analysis shows the imbalanced distribution of ubiquitylated histone H2A (“UbH2A”, which is laid down by PRC1 complex) across the cancer genome (e.g., U2OS osteosarcoma cells (FIG. 19A) as compared to Tig-1 normal fibroblasts (FIG. 19B)). Red bars indicate regions enriched for UbH2A while blue bars denote regions depleted in UbH2A. This genome wide view clearly shows a more “patchy” distribution of UbH2A across the cancer genome compared to the normal cell, with some very large regions of depletion (blue) suggesting sequestration of PcG protein affects in these regions. Thus, the UbH2A status of a cell can also be used to detect the presence of cancer in a sample from a patient.

Our discovery uses the visualization of important epigenetic regulatory proteins to provide a low resolution but “whole genome” synoptic view of their nuclear and genomic distribution, the dramatic nature of which may be less apparent by extraction-based analyses, and provides information on their function even in situations where these key regulatory proteins may not show altered expression levels or functional mutations.

Importantly, many new compounds being investigated for chemotherapy agents (e.g. 5-aza-2′-deoxycytidine and HDAC inhibitors) are known to affect gross epigenetic regulation across the nucleus, and not only at the targeted tumor suppressor gene. It is highly likely that more of these chemotherapeutic agents will produce imbalanced epigenomes in cancer and possibly non-cancer cells, similar to 5-aza-2′-deoxycytidine seen here. Reports suggest that although many patients initially respond well too many of these agents, there are high recurrence rates. We believe that these hallmarks of an imbalanced epigenome will be key in evaluating the effect of these broad range epigenetic inhibitors on normal and cancer cells, the therapeutic outcomes of treatment and recurrence after treatment.

Thus, the presence of large conglomerations of regulatory proteins in cancer cells, such as CAST bodies and their associated Sat II RNA foci or CAP bodies and their associated Sat II DNA foci, as well as changes in the distribution of heterochromatic markers (e.g., ubiquintinated proteins, such as histone H2A, H3K27me, H3K9me2, HP1, H4K20me, loss of H3K4me, loss of H4Ac, DNA methylation (5-mC), and macroH2A), across the genome, are not only common and previously unrecognized “hallmarks” of many cancers, but are robust biomarkers indicative of gross imbalance of epigenetic regulation in the cell. The methods described herein utilize robust biomarkers that can be used to not only diagnose the presence of cancer in a sample from a subject (and thus cancer in the subject), they can also be used to assess whether the cancer is an aggressive cancer. A common thread in the methods described herein is the imbalanced distribution of key chromatin regulators (e.g., PcG proteins and/or MeCP2 proteins, etc.), which is in turn reflected in imbalanced distribution of epigenetic chromatin marks (heterochromatin versus euchromatin), as we demonstrate directly for UbH2A. Knowledge of these changes and how to detect them can be used to provide surveillance, recognition, and proper classification of different cancers, and for designing/evaluating appropriate therapeutic interventions (e.g., avoiding the use of chemotherapeutic agents, such as 5-aza-2′-deoxycytidine, known to produce imbalanced epigenomes).

### EXAMPLES

The following examples are to illustrate the invention. They are not meant to limit the invention in any way.

### Example 1

### Satellite II DNA and Abnormal Nuclear Accumulations of Sat II RNA Mediate Failed Compartmentalization of Master Epigenetic Regulators BMI-1 and MeCP2

### Summary

Epigenomic changes in cancer involve paradoxical gains and losses of heterochromatin within the same nucleus. We report that failed nuclear compartmentalization of polycomb proteins, master regulators of heterochromatin, is prevalent in cancer, and links to locus-specific over-expression of human Satellite II. In cancer, BMI-1 and Ring 1B aggregate in prominent Cancer-Associated PcG (CAP) bodies on the large ˜6 Mb locus at 1q12, which remains silent. In the nucleoplasm low in BMI-1, other Sat II loci express abundant RNA foci; these repeat RNAs accumulate methyl-cytosine binding protein, forming Cancer-Associated Satellite Transcript (CAST) bodies (previously referred to in U.S. 61/507,937 as Cancer-Associated MeCP2 (CAM) bodies). BMI-1 body formation on 1q12, a region commonly hypomethylated in cancer, is induced in normal cells by a DNA demethylating chemotherapeutic. All of these hallmarks of epigenetic dysregulation were readily apparent in vivo, in several breast and other tumors. This study connects novel biology of poorly studied Satellite II, DNA and RNA, to mis-regulation of epigenetic factors in cancer, linked to DNA demethylation at 1q12.

### Highlights:

Large nuclear accumulations of human Sat II RNA are common in many cancer but emanate from only a subset of Sat II chromosomal loci
The normally more dispersed distribution of PcG proteins becomes markedly compartmentalized in cancer, in a manner that mirrors imbalanced expression of different Sat II chromosomal loci
BMI-1 and Ring1B form Cancer-Associated PcG (CAP) Bodies primarily on the large 1q12 Sat II locus, which remains silent, and appears enriched for a distinct sub-set of Sat II repeats
RNA emanates from Sat II loci in the BMI-1 depleted nucleoplasm, which leads to redistribution of another epigenetic regulator, MeCP2, on Sat II RNA foci
Formation of prominent PcG bodies on 1q12 is rapidly induced in normal cells by treatment with a DNA demethylating agent in trial as a chemotherapeutic
Nuclear bodies of Sat II RNA, BMI-1, and MeCP2 are robustly manifest in ascites and primary tumors, including breast and ovarian cancer

### Introduction

In recent years changes in the epigenome have been increasingly recognized as important to tumorigenesis (reviewed in Feinberg and Tycko, 2004; Fraga and Esteller, 2005; Jones and Baylin, 2007). While most attention has focused on silencing of tumor suppressor genes, recent studies recognize a major paradox: this often occurs in the context of broader genomic hypomethylation and/or loss of heterochromatin marks at centric/pericentric satellites (reviewed in Ehrlich, 2009). Centric/pericentric heterochromatin is populated by several classes of satellite sequences. The human satellites (alpha, beta, Sat I, II, III) are comprised of high copy tandem repeats packaged in constitutive heterochromatin and comprise ˜15% of the genome (Richard et al., 2008). In contrast to the 171 bp alpha-satellite repeat at the centromere proper of all human chromosomes, classical Sat II and III are comprised of highly repeated shorter Sat 2 and Sat 3 sequences, respectively, which form larger pericentric blocks on only a subset of human chromosomes. The largest Sat II DNA blocks on chr. 1 and 16 span several megabases of Sat 2 repeats. Sat II is a ˜26 bp degenerate form (Jeanpierre, 1994) of the more conserved 5 bp Sat 3 motif (ATTCC; SEQ ID NO: 1), which comprises the singular large Sat III locus on Chr 9 (Prosser et al., 1986). While a few reports have linked expression of Sat III on Chr 9 to the heat shock response and nuclear “stress bodies” (Jolly et al., 2004; Rizzi et al., 2004), Sat II has long received little attention and remains one of the most poorly-studied prominent features of the human genome.

Despite its abundance Sat II has no known function in normal cells or in disease, although several studies have noted common hypomethylation of Sat II in cancer (Cadieux et al., 2006; Ehrlich, 2009). Satellite heterochromatin has long been believed silent, but recent evidence indicates that certain murine satellites can be expressed at low levels, possibly linked to stress or cell-cycle changes (reviewed in Lu and Gilbert, 2007; Probst and Almouzni, 2007; Vourc'h and Biamonti, 2011). The fact that satellite sequences were so long considered transcriptionally silent is testimony to the fact that their expression has been difficult to detect using standard molecular techniques. However, RNAs tightly associated with chromatin or nuclear structure may be more amenable to analysis in situ; this also preserves molecular information in chromosomal and structural context, which proved key to most findings presented here.

Polycomb group (PcG) proteins, are a family of master epigenetic regulators that control most early developmental pathways, primarily through repressive chromatin modifications (reviewed in (Sparmann and van Lohuizen, 2006), and also function in the formation and maintenance of constitutive peri/centric satellite heterochromatin. Polycomb repressive complex 2 (PRC2) includes the EZH2 protein, which introduces trimethylation of histone H3 lysine 27 (reviewed in Valk-Lingbeek et al., 2004), whereas PRC1 includes BMI-1 and RING1B, which promotes histone ubiquitination (reviewed in Niessen et al., 2009), DNA compaction (Eskeland et al., 2010) and other modifications. In Drosophila embryos “PcG bodies” are believed to contribute to gene silencing via differential organization and access of gene loci to these concentrated repressive factors (Bantignies et al., 2011). In mammalian cells, prominent PcG bodies (with BMI-1 and RING1B) have also been described and are widely considered to be part of normal nuclear structure. BMI-1 is a key component of PRC1 and is essential for self-renewal of neuronal and hematopoietic stem cells, as well as suppression of the tumor suppressor locus Ink4a/Atf (Jacobs et al., 1999). Although BMI-1 over-expression has been linked to cancer progression (reviewed in Valk-Lingbeek et al., 2004), other evidence indicates a more complex relationship such that over-expression can correlate with a good prognosis in breast cancer (Pietersen et al., 2008). Thus the role of BMI-1 in cancer is currently intensively studied but unresolved (Glinsky, 2008; Lukacs et al., 2010; Riis et al., 2010).

The dichotomy regarding TS gene silencing versus broader breakdown of heterochromatin components (Pageau et al., 2007), suggests to us an imbalanced nuclear epigenome, the basis for which is unknown. Studies from our lab and others have shown that in normal somatic cells, specific genomic loci reside in distinct nuclear sub-compartments enriched for specific metabolic and regulatory factors (Hall et al., 2006; Misteli, 2000, 2004). This nuclear compartmentalization is increasingly recognized as an important contributor to the overall epigenetic program of particular cell types. Non-coding RNAs are being recognized for their normal role in recruitment of epigenetic regulators (Hall and Lawrence, 2011; Koziol and Rinn, 2010; Masui and Heard, 2006) as well as the structural underpinning for nuclear bodies (Clemson et al., 2009; Wilusz et al., 2009). In addition, repeat RNAs have been shown to underlie pathology in certain triplet repeat diseases (Osborne and Thornton, 2006). In this study, we provide evidence that key epigenetic regulators show aberrant compartmentalization within cancer nuclei that is intimately connected to localization on certain Sat II loci and to inappropriate expression of Sat II RNA from others.

This study began with a broad survey of Cot-1 repeat RNA expression and distribution in human interphase nuclei. While competition with unlabelled Cot-1 DNA (repetitive genomic fraction) (Britten and Kohne, 1968) is often used to suppress hybridization to repeats, here we labeled human Cot-1 DNA as a probe to examine the distribution of transcripts from the repeat genome by RNA FISH. We previously showed that hybridization to Cot-1 RNA provides a convenient assay to evaluate chromosome inactivation within nuclei (Clemson et al., 2006; Hall et al., 2002), and also reveals breakdown of the peripheral heterochromatic compartment in cancer (Pageau et al., 2007b). However, this study began when large localized foci of Cot-1 RNA were initially observed and then shown to be exclusive to cancer cells.

### Results

Expression of the Cot-1 Genomic Fraction Reveals Large Nuclear Foci of Repeat RNAs in Cancer but not Normal Cells:

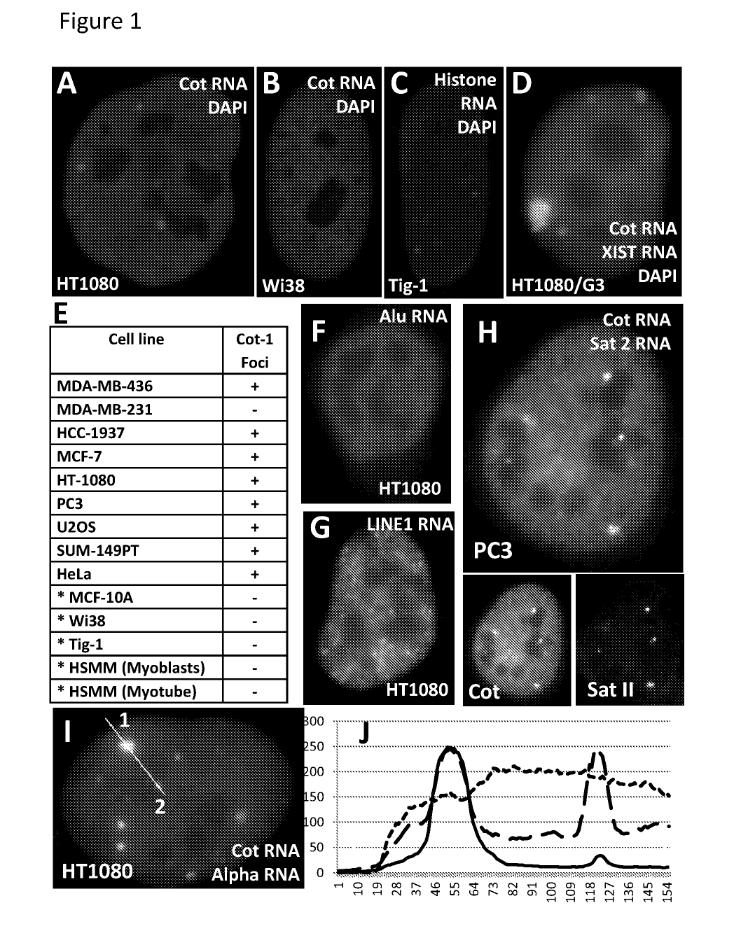

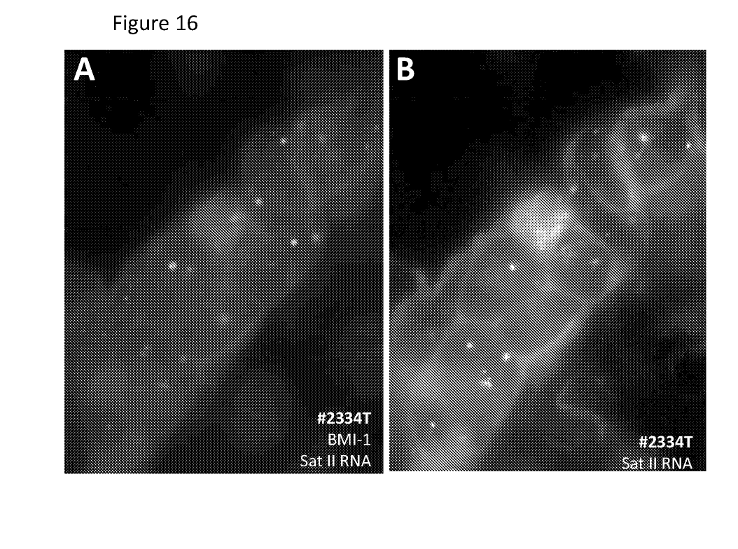

In situ hybridization to repeat RNAs using a Cot-1 probe consistently produces a substantial disperse nucleoplasmic signal in all mammalian cells examined with essentially no cytoplasmic signal (FIG. 1B). However, we noted that some cell lines also contained multiple prominent localized concentrations of repeat RNA in nuclei (FIGS. 1A and 1D). The typically large (˜0.4-1 micron) very bright foci suggest abundant localized repeat RNA, as illustrated by comparison to exceptionally bright nuclear RNA signals generated by XIST RNA (which paints the whole inactive X chromosome) (FIG. 1D) or the more typical RNA signal seen with transcription foci from individual genes (e.g. histone RNA) (FIG. 1C). Since not all cell samples contained Cot-1 RNA foci, expanded analysis of numerous cell lines revealed they were present in most of the neoplastic cell lines examined (FIG. 1E and FIGS. 16A and 16B), but none of several normal, non-neoplastic cell lines. This suggests a common dysregulation of some component(s) of the “repeat genome” in cancer.

Cot-1 RNA Nuclear Foci are Primarily Satellite II RNA, which is Undetectable or Negligible in Normal Cells:

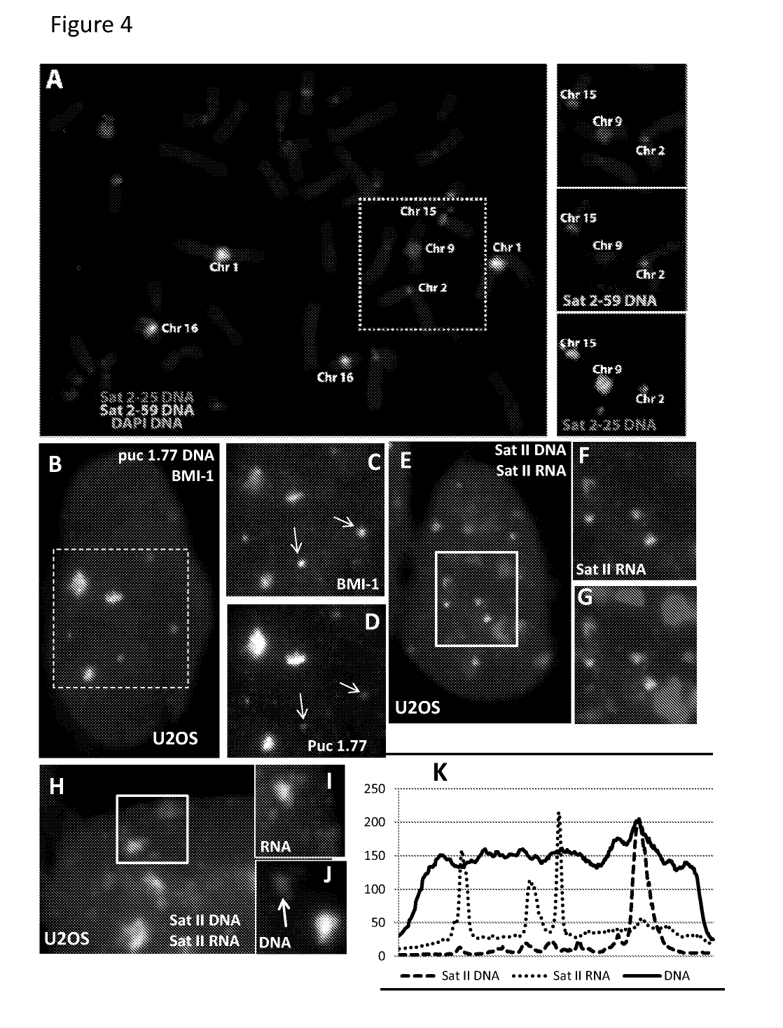

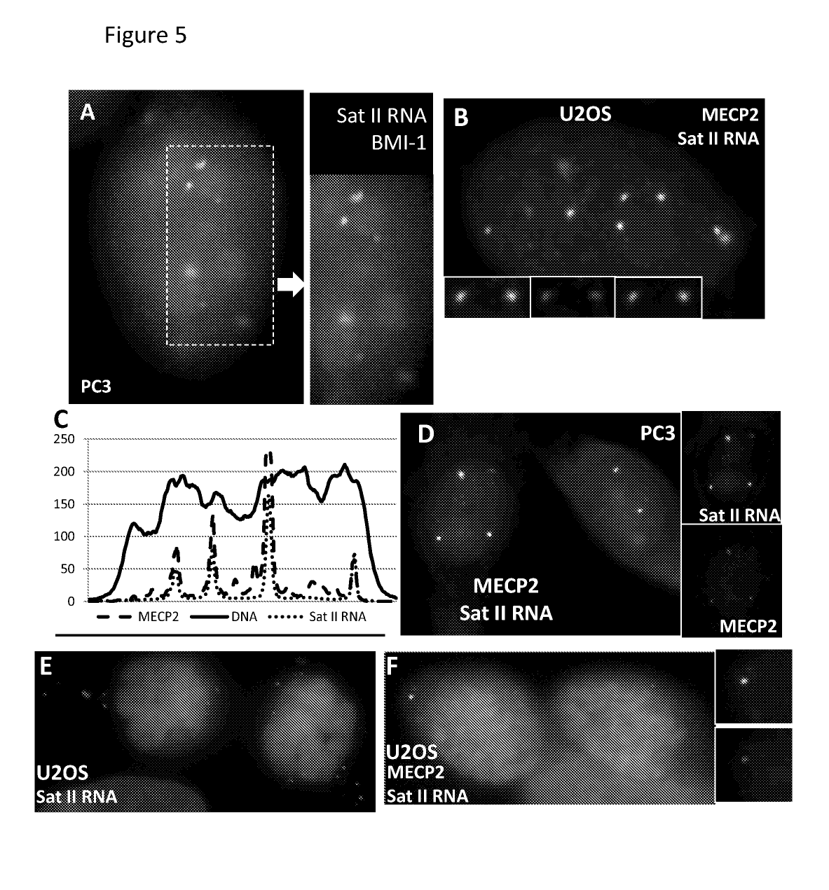

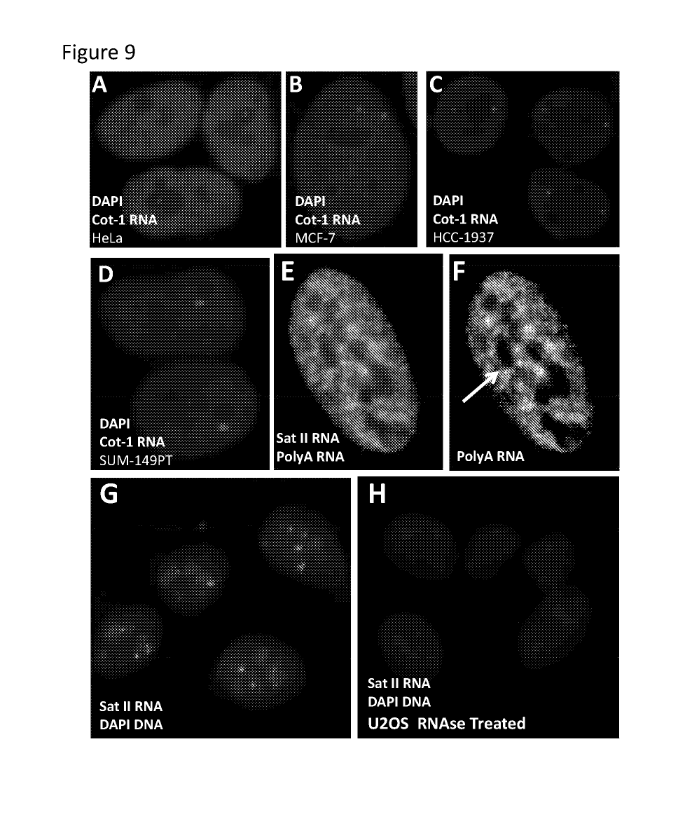

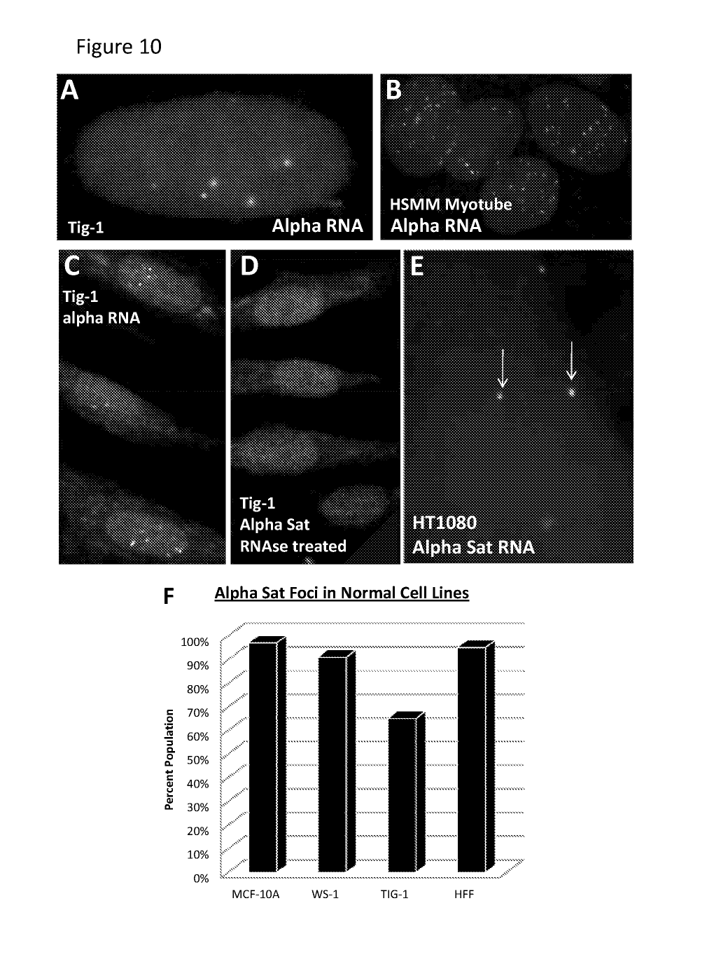

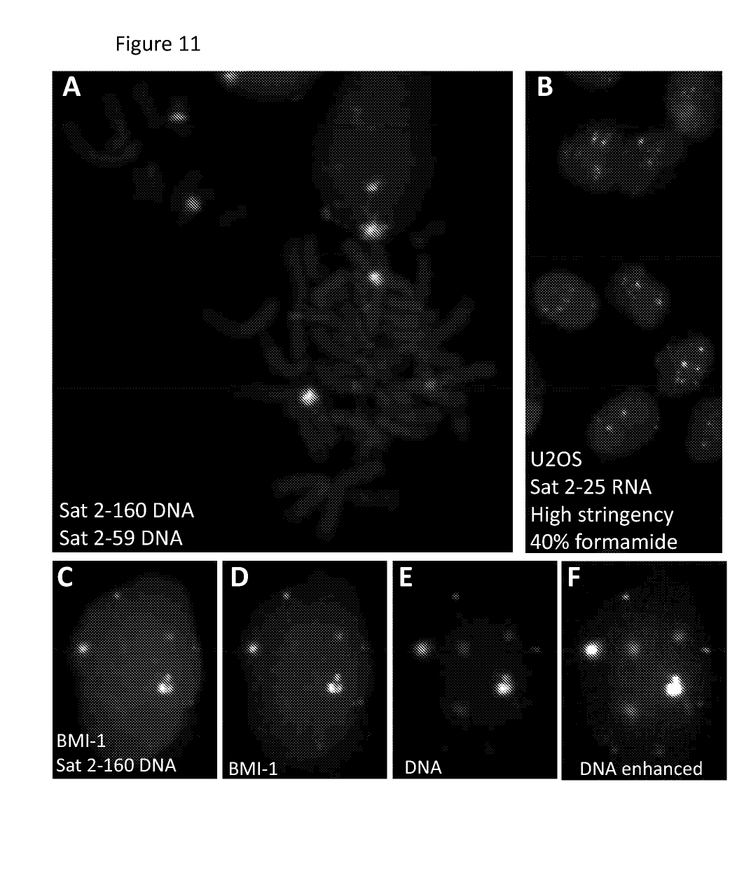

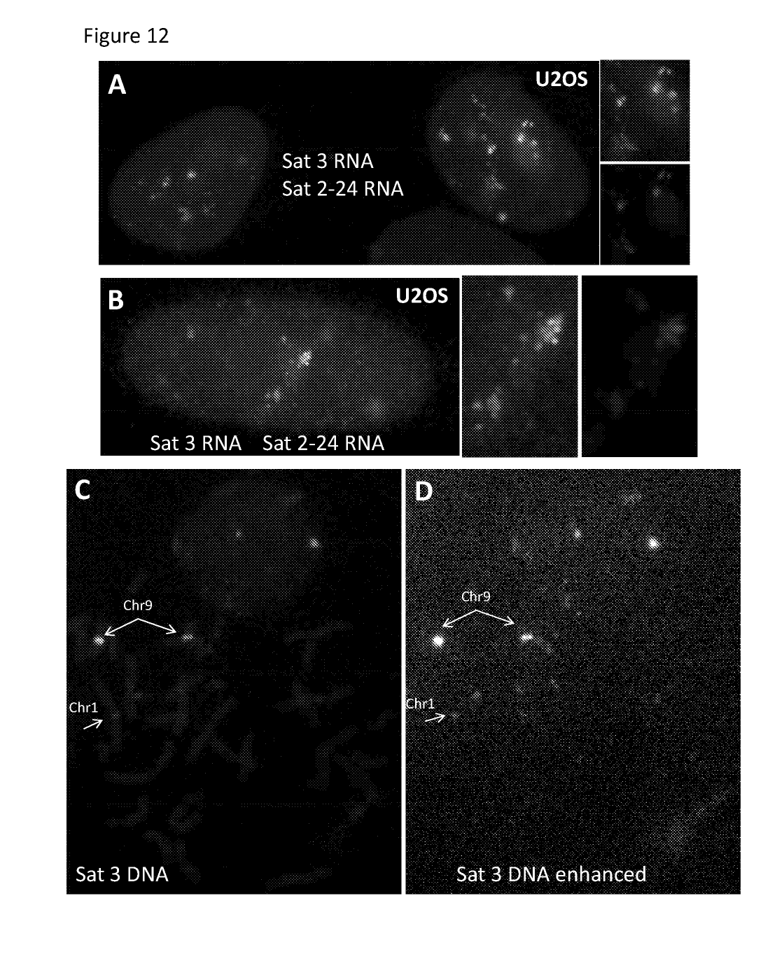

Cot-1 DNA is a complex probe containing several major classes of repeats. Therefore we used probes to specific repeats to better define the content of these large Cot-1 RNA foci. RNA hybridization with probes for LINE (L1) and SINE (Alu) repeats generally did not detect localized concentrations of RNA (FIGS. 1F-1G), and alpha-satellite RNA was also not coincident with Cot-1 RNA foci in most cancer lines, although it did label a subset of Cot-1 RNA foci in HT1080 (FIGS. 1I-1J) and MDA-MB-436 cells. In contrast, the majority of Cot-1 RNA foci in most cancer lines was comprised of Sat II RNAs (FIG. 1H). Table 2 (below) summarizes that eight of twelve cancer cell lines, representing different types of cancers, exhibit prominent nuclear foci of Sat II RNA in the vast majority (70-100%) of cells. Several observations support that these are RNA signals: 1) hybridization without denaturation of cellular DNA, 2) removal with RNAse (FIGS. 9G and 9H and FIGS. 10C and 10D) or NaOH treatment 3) absence in some cell lines, and 4) absence on mitotic chromosomes but frequent detection in the cytoplasm of mitotic cells (FIG. 10E and FIGS. 5E and 5F). Evidence also suggests this is single-stranded and non-polyadenylated RNA (FIGS. 10E and 10F), and shows predominantly only a single direction of synthesis with little expression from the “reverse” strand. We utilized a number of different Satellite probes (see FIGS. 11A-11F and FIGS. 12A-12D, and methods) including four different Sat II probes. These included standard Sat II oligos as well as a highly sensitive 24 nt LNA oligo (Sat 2-24) which maximized detection of Sat 2 family sequences at low stringency. As shown in FIG. 4A, hybridization to metaphase chromosomes with the Sat 2-24 LNA oligo detects large Sat II loci in Chr 1 and 16 pericentromeres and small signals on several other chromosomes, consistent with a prior report (Silahtaroglu et al., 2004), whereas the Sat 2-59 probe is more restricted to Chrs 1 and 16 and a few other loci (Chrs 2, 15, 10). (We note that the LNA probe can detect the related Sat III locus on Chr 9 at lower stringency). Both probes detect similar Sat II RNA foci in cancer cell nuclei, however the Sat 2-24 LNA probe produced especially robust signals at both lower and higher stringency (FIG. 11B).

It was important to address whether normal human cells show significant expression of Sat II and alpha-Sat RNA using specific probes that are more sensitive for a given sequence. In fact, we surprisingly found that nuclear foci of alpha-satellite RNA are readily detected by FISH in normal cells (FIGS. 10A-10F). This illustrates the high sensitivity of in situ hybridization for nuclear embedded RNAs, but contrasts to results for Sat II RNA, as further detailed below. Since the difference between alpha-satellite RNA expression in normal and cancer cell lines was not as marked as for Sat II RNA, our focus in the rest of this study is on Sat II RNA.

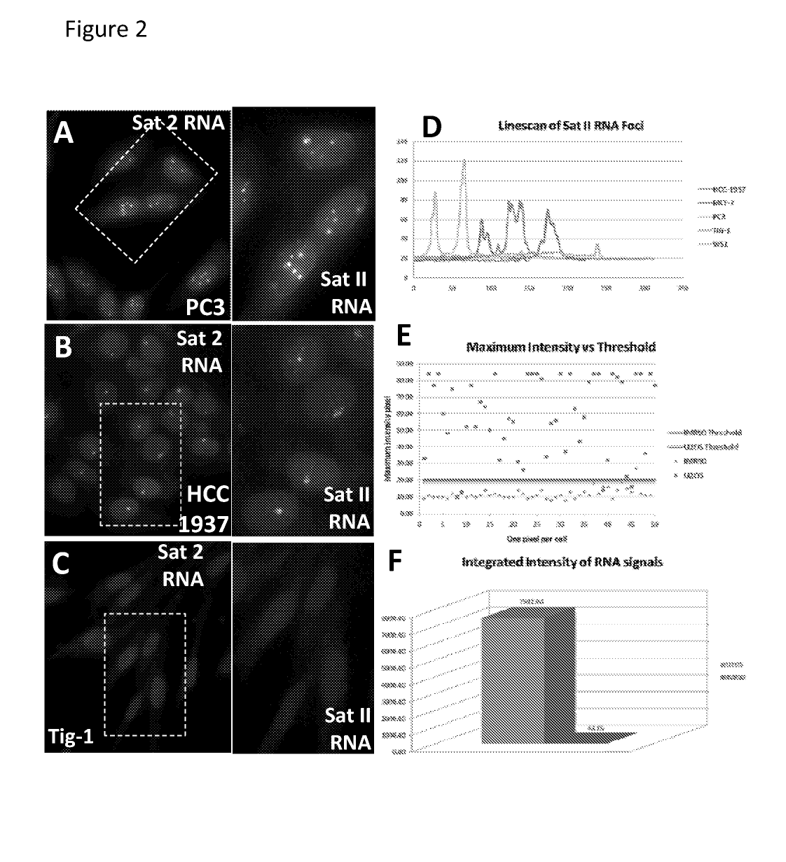

The difference between Sat II RNA expression in cancer versus normal cells was easily discerned by eye, was scored consistently by multiple investigators, and moreover, could be quantified by digital microfluorimetry (FIGS. 2A-2F). Unlike what was seen with alpha-sat, normal cells were mostly negative for Sat II foci, with only a very small subset showing one or two tiny fluorescent pinpoints that could be detected using digital imaging but were undetectable or barely detectable by direct visualization (FIG. 2C). The linescan in FIG. 2D quantifies this difference in single cells, while FIG. 2E shows that a straightforward measurement of highest pixel intensity in a population of cells clearly distinguishes cancer from normal. Further quantification of total RNA signals in 10 random cells/sample (see methods) indicates Sat II RNA in U2OS cancer cells is at least ˜175 fold greater than in normal cells (FIG. 2F). Thus, prominent aberrant foci of Sat II RNA are a unique “signature” of cancer cells, which can mark even a single cancer cell as distinct from normal (see tumor tissues below), by direct visual analysis or quantitative digital microscopy.

Cancer Associated Polycomb (CAP) Bodies Form on Sat II Loci at 1q12 in Neoplastic but not Normal Cells:

To gain insight into potential causes of Sat II expression in cancer cells we examined the proteins known to regulate satellite heterochromatin, the repressive Polycomb Group (PcG) proteins. PcG proteins, including BMI-1, are also linked broadly to developmental gene regulation and stem-cell self-renewal, and are increasingly implicated in cancer pathogenesis. The PRC1 complex proteins, BMI-1 and Ring1B, were of particular interest since these were reported to form Polycomb bodies (PcG bodies) and localize to Sat II loci, particularly the very large (6 Mb) Sat II block at 1q12 (Saurin et al., 1998). Mammalian PcG bodies were initially described as normal nuclear structures (Saurin et al., 1998) and are currently considered and studied as such (reviewed in (Bernardi and Pandolfi, 2007; Spector, 2006)). However, when we initially examined BMI-1 staining in a panel of various cell types, there was a key difference between normal and cancer cells.

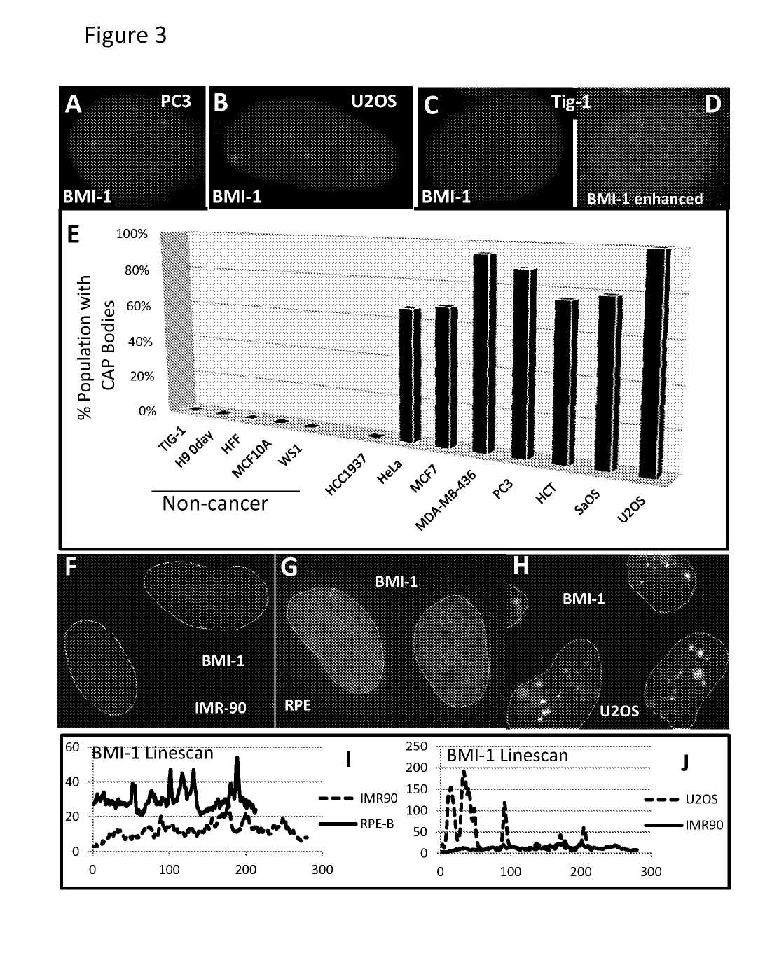

We found that BMI-1 staining brightly labeled a few very prominent nuclear bodies in most cells in 7 of 8 neoplastic lines, which were not seen in non-neoplastic cells. For example, as seen in FIGS. 3A-3J, over 90% of U2OS cells exhibit large (0.4-1.5 microns) discrete bodies, which contrast sharply with a much darker nucleoplasm. In contrast, although the normal staining in non-neoplastic cells can vary somewhat between cell types, they have a more uniform granular or particulate BMI-1 distribution throughout the nucleoplasm, but not the same large, prominent bodies seen in cancer cells. The difference between normal and cancer cells is exemplified further in FIGS. 3F-3J by comparison of U2OS cells to IMR-90 fibroblasts or telomerase immortalized RPE cells, which have a small subset of cells with 1-2 small dim BMI-1 “punctuates.” The contrast ratio for the brightest PcG punctate in RPE cells was ˜4:1, whereas this is conservatively 20:1 for the 6-8 larger PcG bodies in U2OS cells (see quantitative linescans, FIGS. 3I and 3J). The high contrast ratio between the BMI-1 in bodies versus the nucleoplasm makes the point that this is a markedly different distribution in cancer, not just higher overall levels. Even if the overall level of the protein is higher, as illustrated for U2OS BMI-1 piles up sharply at a few sites, while the nucleoplasm (where most chromatin resides), has much lower levels. Thus, the normal nuclear genome has more uniform access to these factors, whereas cancer nuclei have grossly aberrant compartmentalization of these master epigenetic regulators, with a few “hot spots” of concentrated factors and a generally more restricted access through much of the nucleoplasm.

As mentioned above, PcG bodies, which are repressive proteins, have been reported to localize to the large Sat II block on 1q12 (initially studied in HT1080 cells, a fibrosarcoma cell line) (Saurin et al., 1998). We confirm that the PcG bodies previously reported to localize to 1q12 are the same large aberrant PcG bodies studied here. Using dual labeling with 1q12 specific probes (puC 1.77 kb and Sat2-160 bp) and BMI-1 in U2OS and PC3 cells, we show that these large PcG bodies clearly and consistently (˜100%) co-localize with the 1q12 DNA signal in these cancer cells (FIGS. 4B-4D and FIGS. 11C-11F). Thus, these large, prominent PcG accumulations, which exhibit a high contrast ratio with the nucleoplasm and preferentially “cap” the Sat II locus at 1q12, are a hallmark of cancer cells, and are not a normal nuclear structure. To avoid confusion with the smaller, more numerous and widely dispersed particulate PcG foci in normal human cells, often referred to as “PcG bodies” (Grimaud et al., 2006; Saurin et al., 1998), we refer to the less numerous and larger conglomerations of PcG proteins at 1q12 in cancer cells as “CAP” bodies, for “cancer associated PcG” bodies.

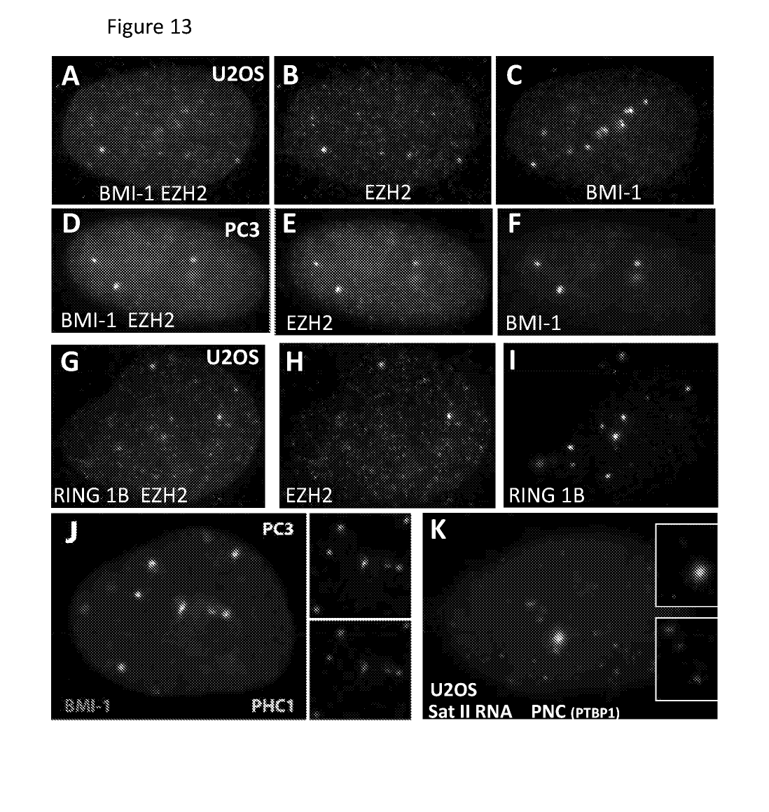

While most of our analyses utilized BMI-1 staining, we confirmed that RING 1B and Phc1, also in the PRC1 complex, concentrate sharply in the same CAP bodies (FIGS. 13A-13J). We also briefly examined EZH2 (in the PRC2 complex) and confirmed that this did not overlap either BMI-1 or RING 1B bodies in U2OS cells (Hernandez-Munoz et al., 2005), although it was somewhat elevated there in PC3 cells (FIGS. 13A-13J). However, EZH2 remained higher throughout the nucleoplasm, in contrast to the nucleoplasmic depletion of the PRC1 components (BMI-1, Ring1B, Phc1).

Imbalanced Expression of Sat II Loci on Different Chromosomes Inversely Correlates with Aberrant Compartmentalization and Sequestration of PcG Proteins:

Sat II RNA over-expression could reflect failed maintenance of Sat II heterochromatin throughout the entire cancer genome. However, given the imbalanced nuclear compartmentalization of repressive polycomb proteins shown above, it was important to assess if all Sat II loci express RNA, and if not, determine if there was a random or non-random relationship between locus expression and PcG protein nuclear distribution. A priori we considered two alternate possibilities for a potential relationship between PcG proteins and Sat II RNA distributions. Since ncRNAs can recruit PcG proteins including BMI-1, Sat II RNA foci might emanate from the largest Sat II loci in the pericentromeres of Chrs 1 and 16, and induce PcG proteins to form CAP bodies there. Alternatively, the abundant PRC1 factors in CAP bodies on 1q12 and 16q11 may maintain repression of Sat II at these loci, while in the same nucleus relative depletion of these repressive factors from the rest of the nucleoplasm could contribute to aberrant expression from other Sat II loci.

The number of Sat II RNA foci varied in a manner characteristic for a given line (see Table 2), but this did not correlate with ploidy differences (see legend, Table 2), suggesting that only a subset of Sat II loci are expressed. To determine this directly, we used a sequential hybridization strategy to RNA and then to DNA (Smith et al., 2007; Xing et al., 1995) to visualize these simultaneously in two different colors (using the same Sat 2-24 sequence as probe) (see methods). As apparent even in U2OS, which has the most RNA foci of any tumor line, not all Sat II DNA loci are associated with an RNA signal, whereas RNA foci usually abut or partially overlap a DNA signal (FIGS. 4E-4K). Interestingly, the RNA foci typically emanated from the very small or medium Sat II DNA loci, but consistently not from the largest Sat II DNA loci (on 1 and 16). In fact, since Sat II RNA and CAP bodies are largely mutually exclusive (0% overlapping, 6% adjacent and 94% no association) (FIG. 5A), this reveals not only that mis-regulation of different Sat II loci is not equal, but further demonstrates a clearly inverse relationship to the nuclear organization of PcG proteins in bodies, predominantly at 1q12 and 16q11. While these large Sat II domains which amass PRC-1 CAPs remain silent, large nuclear foci of Sat II RNA emanate from much smaller Sat II DNA loci in the nucleoplasmic compartment sharply lower in PRC1 proteins. Thus, these results show a marked imbalance in expression/repression of Sat II loci on different chromosomes in cancer, which in turn mirrors the aberrant nuclear compartmentalization of these key epigenetic regulatory factors.

MeCP2 Accumulates with the Sat II RNA Foci and not with Sat II DNA at 1q12 Associated with CAP Bodies:

While this aberrant compartmentalization of epigenetic factors was previously unknown in cancer, abnormal DNA methylation has been intensely studied, and it would be important if our studies would reveal a link between these two major areas of epigenetic regulation. Given that the 1q12 Sat II locus accumulates PRC1 and is repressed, we considered it may be hypermethylated. On the other hand, substantial literature reports that Sat II at Chr 1 and 16 is commonly hypomethylated in many cancers. Thus we examined whether antibodies to MeCP2, a methyl-DNA binding protein, labeled the 1q12 domain associated with the PRC1 CAP bodies in cancer cells. Staining in U2OS cells revealed that MeCP2 sharply accumulates in several bright nuclear foci (FIG. 5B), distinct from the more dispersed and punctuate distribution in normal cells. This appeared to suggest methylation “hot-spots” in cancer nuclei. Since these foci were not unlike the distribution of BMI-1 in cancer, it was initially expected that co-staining would show a relationship. However we found that BMI-1 (or Sat II DNA) and MeCP2 foci do not overlap, but rather are mutually exclusive. Since these large MeCP2 foci in cancer cells also appear similar to Sat II RNA foci, we hybridized to Sat II RNA in cells also stained for MeCP2, and, remarkably, these two strictly co-localized. As shown in FIGS. 5B-5D for U2OS and PC3, in most cells every Sat II RNA focus coincides precisely with an MeCP2 focus. Note that, as shown above (FIGS. 4E-4G and 4K), Sat II RNA foci often do not precisely coincide with or “paint” their associated Sat II DNA loci, but accumulate mostly adjacent to the DNA signal; thus the precise correlation of MeCP2 with RNA foci is not due to a relationship to the DNA. The surprising association of MeCP2 with Sat II RNA is further indicated by their precise co-localization in small punctate cytoplasmic signals released from nuclei in mitotic cells (FIG. 5F). While MeCP2 is mostly studied as a DNA binding protein, several studies have reported it can also bind RNA, in vitro and in vivo, and impacts mRNA processing and splice site recognition (Hite et al., 2009; Jeffery and Nakielny, 2004; Long et al., 2010; Young et al., 2005). In addition, some RNAs, such as tRNAs, contain 5-methylcytosine, which can impact RNA stability (Motorin et al., 2010). In any case, the precise accumulation of MeCP2 with Sat II RNA foci suggests that these abundant satellite repeat RNAs impact the distribution of this methyl-DNA binding protein, and potentially other factors involved in epigenetic regulation of the nuclear genome, as further considered in the Discussion.

CAP Bodies Accumulate on 1q12 in Normal Fibroblasts Treated with a Global DNA Demethylating Agent in Development as a Chemotherapeutic:

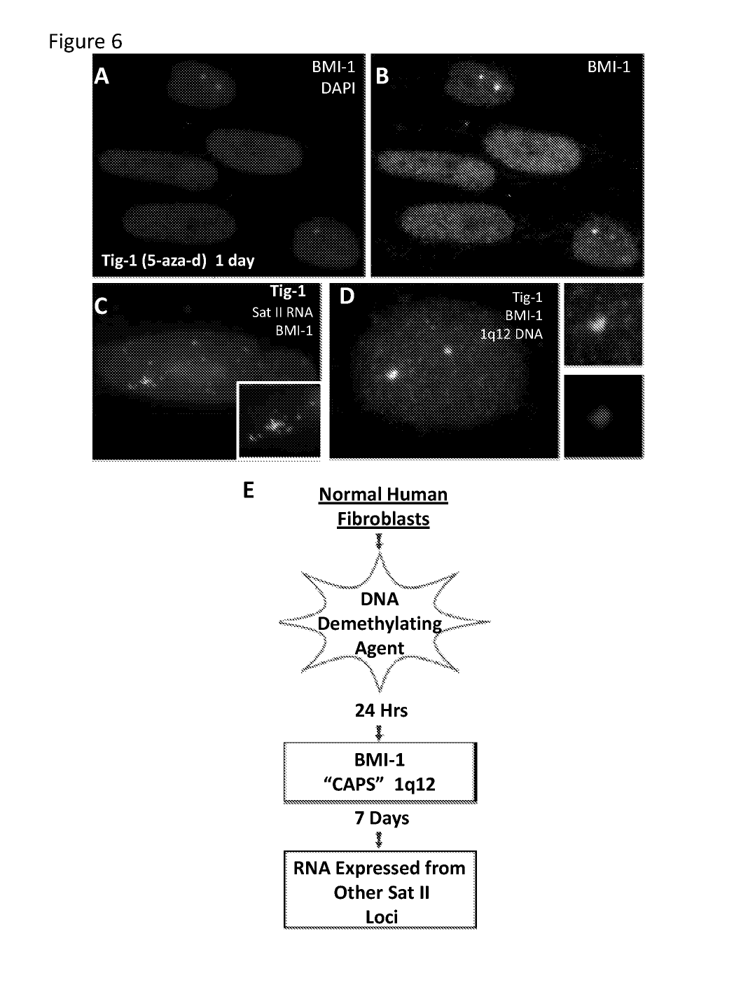

The fact that MeCP2 does not localize to 1q12 is consistent with reported Sat II hypomethylation in many cancers, particularly breast, ovarian, Wilms tumor, multiple myeloma, glioblastoma, among others (reviewed in (Ehrlich, 2009). In fact, it has been reported that the 1q12 satellite is the region most susceptible to hypo-methylation in tumors, although it is not clear that the assays used could discriminate Sat II at 1q12 from other Sat II loci. Since DNA methylation changes are extensively documented in cancer, it would be important if these had an impact on the distribution of PcG proteins. To investigate this, we treated normal human fibroblasts with 5-aza-2′-deoxycytidine (5-aza-2d or decitabine), a pharmacologic inhibitor of DNA cytosine methylation, in limited clinical use and in trials as a chemotherapeutic for other cancers (reviewed in (Kelly et al., 2010). 5-aza-2′d has also been shown to effectively demethylate Sat II on Chromosome 1 (Ji et al., 1997), allowing us to test the possibility that this would in turn impact BMI-1 distribution in normal cells. Remarkably, within 24 hours of a single treatment, a marked accumulation of PRC1 components (BMI-1 and Ring1B) was seen at two large “bodies” within nuclei of ˜15% of primary human fibroblasts (consistent with the effect requiring transition through S-phase); these were similar in size and shape to the 1q12 DNA signal (FIGS. 6A-6B). Subsequent hybridization with the 1q12 specific DNA probe (puc 1.77 kb) directly confirmed that PRC1 PcG proteins are induced to “cap” the 1q12 Sat II loci in normal cells, shortly after treatment with 5-aza-2d (FIG. 6D). Longer treatment increased both the number of CAP positive cells (Day 1=15%, Day 3=52%, Day 8=80%), as well as the number of CAP bodies per cell (from ˜2 on Day 1, to ˜4 on Day 8). Importantly, aberrant Sat II RNA foci also appeared with longer term 5-aza-2d treatment, and importantly from sites distinct from 1q12 bearing the accumulated BMI-1 bodies (FIG. 6C). Aberrant Sat II expression was not seen in control or 1 day treated cells, was rare on day 3 and in ˜5-10% of cells by day 8. These findings reveal significant mechanistic insight into why PcG proteins so markedly accumulate at 1q12 in cancer but not normal cells, and provide important evidence of a link between two intensely studied areas of cancer epigenetics, DNA methylation and polycomb proteins. Results indicate that loss of cytosine methylation at 1q12 is not only correlated with, but precedes and leads to abnormal PRC1 binding. Additionally, these results, in normal diploid human cells, provide new perspective (and a potential biomarker) for the impact of chemotherapeutic agents on the broader epigenome of patient cells.

While hypomethylation at 1q12 leads to BMI-1 body formation there, a related question arises as to why PRC1 proteins do not aggregate proportionally on the other Sat II loci, since RNA expression from other loci indicates that they are likely also hypomethylated. In the course of these experiments we tested four different Sat II probes (see methods), which suggested that distinct sub-types of Sat II DNA correlate with CAP body formation. While details are in the methods, in sum the results suggest enrichment for different Sat 2 sequence sub-types on different Sat II chromosomal loci, which correspond to the distribution of CAP bodies. Sat 2 probes derived from the 1q12 sequence, which have a more restricted distribution on Chrs. 1 and 16 (FIG. 11A), exhibit good coincidence with CAP bodies (FIGS. 4B-4D and FIGS. 11C-11F), and do not detect appreciable RNA. Thus, a Sat 2 repeat sub-type with strong affinity for PcG complexes appears to populate Sat II loci on Chrs 1 and 16, but not other secondary Sat II loci; this may explain the higher sensitivity of 1q12 to hypomethylation and accumulation of PRC-1 proteins. While a fuller characterization of Sat II sub-types is beyond the scope of this study, these findings highlight a complex organization of human satellite II, and most importantly, demonstrate that certain DNA sequences within the human Sat II family underlie compartmentalization of epigenetic regulators and aberrant nuclear bodies, linked to hypomethylation of these repeats in the cancer epigenome.

Aberrant Satellite RNA Foci, CAP Bodies, and “CAST” Bodies in Tumors In Vivo:

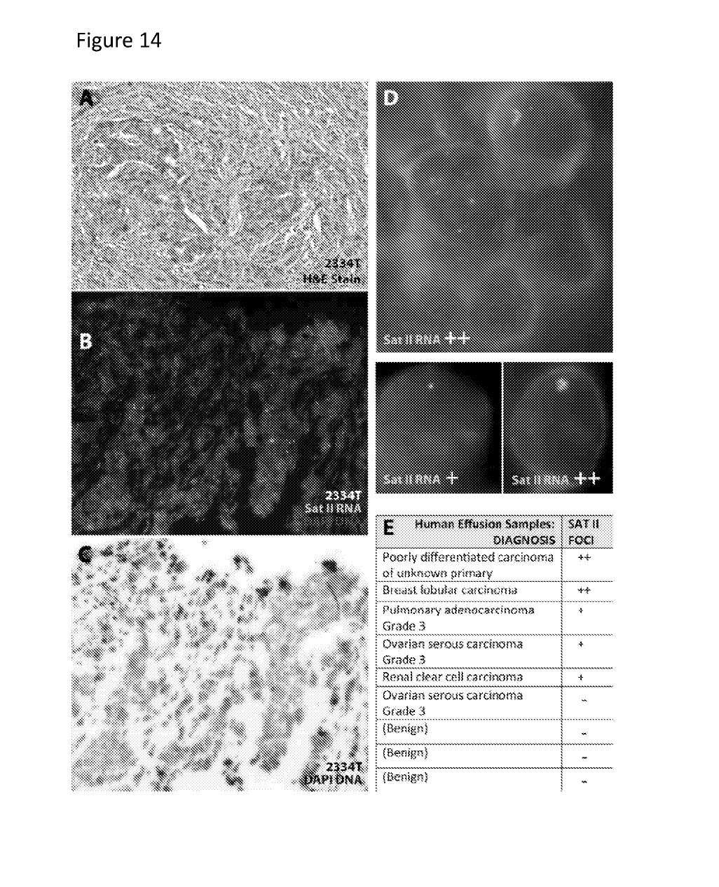

Since Sat II RNA foci are not in normal cultured cells, they cannot arise only as a consequence of cell culture. Nonetheless, a key question is whether these changes arise in vivo and would be detectable directly in tumor tissues. We began with abdominal and pleural effusions from 10 patients. Despite a high auto-fluorescence of these initial preparations, Sat II RNA foci were evident in five of nine samples examined by two blinded investigators (FIGS. 14D-14E). Of the four negative samples, three were from patients without evidence of cancer based on cytopathological analysis in clinical follow-up, whereas all other cases were from patients with malignant effusions. Sat II RNA foci were not seen in all cells of positive cases, but were restricted to cells primarily in clusters, showing nuclear enlargement and irregularity consistent with malignancy.

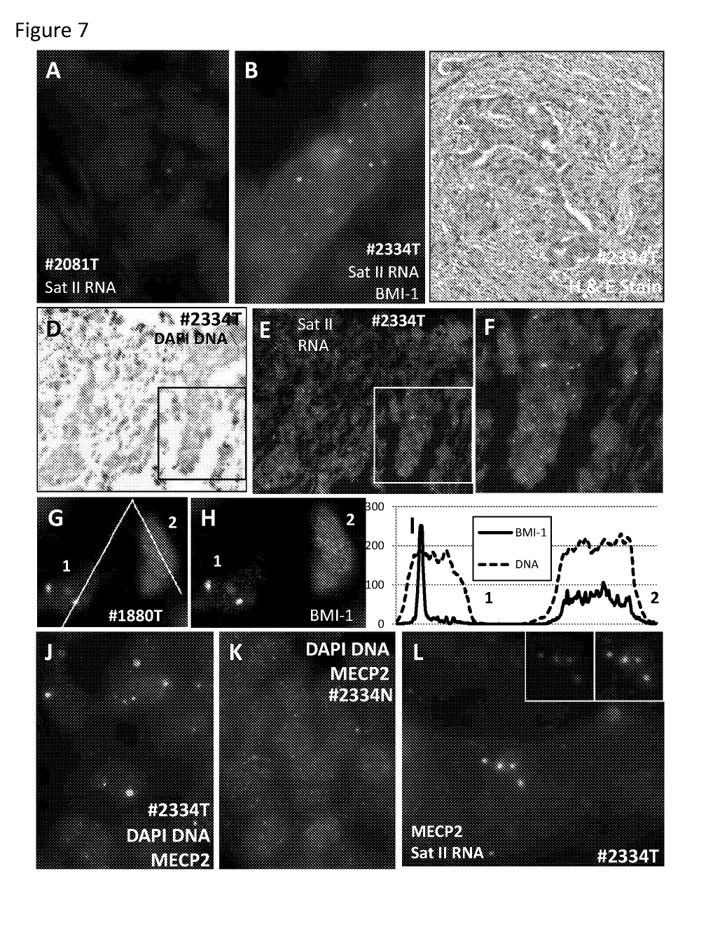

Next, we examined several primary solid tumors in cryostat sections (which are readily amenable to fluorescence analyses), obtained through the UMMS tissue bank, with some matched normals. Given that RNA preservation in such pathology samples can be a challenge, we used FISH to poly A RNA as a positive control and tested three different fixation protocols to determine the most effective one (see Methods). The poly A RNA preservation varied with the sample and was generally poor to moderate as compared to cultured cells. Nonetheless, the first tumor sample examined (Block #2334T) displayed remarkably robust and prevalent Sat II RNA foci (FIGS. 7B and 7C), apparent even at low (10×) magnification (FIGS. 7D-7F and FIGS. 14A-14C). This ductal breast carcinoma had a very high frequency of cells with typically 1-3 prominent Sat II RNA foci; these cells clustered around ducts and displayed other nuclear and morphological features of cancer. In contrast, this was not seen in either the matched normal sample (#2334N), other normal breast samples, nor in other normal cell types within the tumor sample. As shown in Table 3, five of six primary tumor samples examined (by two independent investigators scanning at least 500-1000 cells per sample) contained cells positive for Sat II RNA over-expression (FIG. 7A), unlike the matched normal samples. Similar to the human effusion samples the single negative tumor sample was also benign. The Sat II RNA was detectable even in tumors in which poly A RNA detection was sub-optimal, suggesting the Sat II RNA is stable and/or potentially even more abundant than it appeared.

Based on above results with cultured cells, we hypothesized that CAP bodies would be in the same tumor cell nuclei with Sat II RNA foci, but in separate nuclear locations. As illustrated in FIG. 7B for the 2334T breast ductal carcinoma, this is precisely what was seen. Sat II RNA foci were apparent in 80% of nuclei that exhibited CAP bodies, further supporting a relationship between them. As expected the matched normal tissue had particulate nucleoplasmic BMI-1 staining but not the prominent CAP bodies. The normal nucleoplasmic levels of BMI-1 staining showed some fluctuation between tissues; for example, in the 2312N (normal pancreas) the generally high punctate staining in normal cells may preclude analysis of CAP bodies in this tissue. Importantly, as illustrated in a renal tumor sample (#1880T) (FIGS. 7G-7I), the presence of one or more prominent CAP bodies was often accompanied by marked sequestration of BMI-1 from the rest of the nucleoplasm.

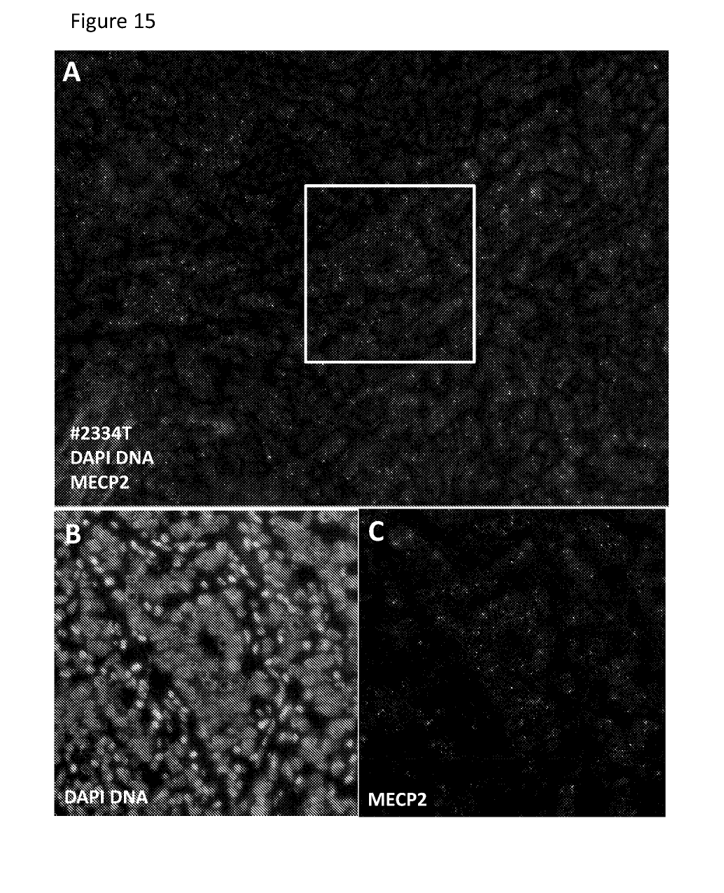

Finally, we also confirmed that the aberrant MeCP2 foci shown above in several cancer lines also occur in vivo. As shown in FIG. 7J for the breast tumor #2334T (and larger tissue image in FIGS. 15A-15C), many cells exhibit a striking pattern of one or a few large, round bright MeCP2 “bodies”, often contrasting with a much darker nucleoplasm. Matched normal tissue (#2334N) had a higher nucleoplasmic stain with a somewhat variable punctate pattern (FIG. 7K), but not large bodies against a dark nucleoplasm. Thus, this dramatic accumulation of MeCP2 at just a few sites we refer to as “Cancer Associated Satellite Transcript” (CAST) bodies. Importantly, these in vivo CAST bodies were separate from BMI-1 bodies but precisely overlapped Sat II RNA foci (FIG. 7L), further corroborating the results above suggesting that MeCP2 proteins localize to Sat II repeat RNAs in cancer.

### Discussion

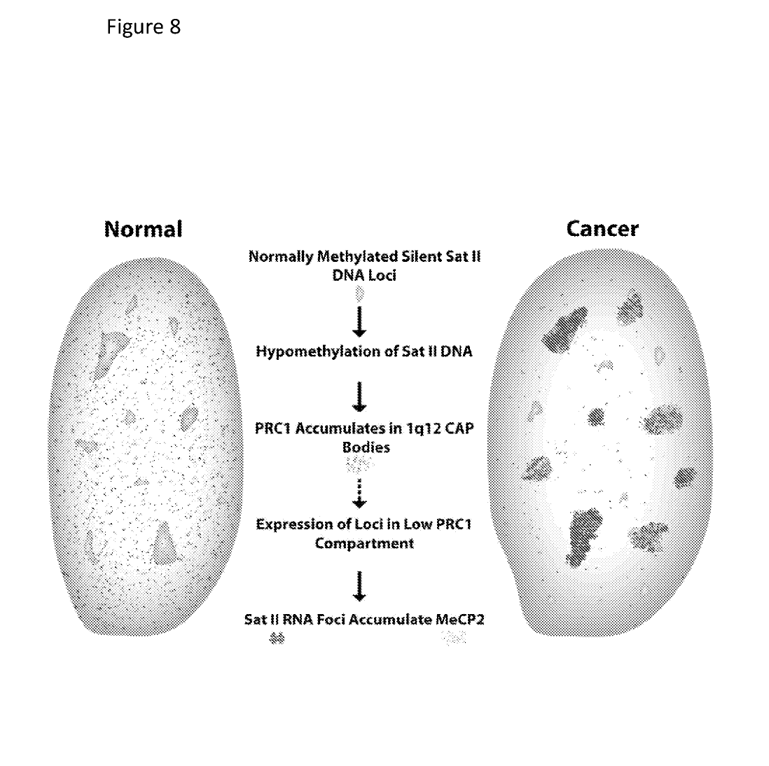

As summarized in the model in FIG. 8, this study demonstrates several new fundamental properties of cancer cells which collectively provide novel and fundamental insights into epigenetic dysregulation in cancer. It points to the unanticipated importance of human satellite II DNA and RNA in epigenetics and disease, via the capacity of high copy repeats to impact the nuclear distribution of regulatory factors. Importantly, despite many studies noting hypomethylation of Sat II repeats in cancer (particularly at 1q12), we demonstrate for the first time that this connects to marked change in nuclear compartmentalization of PcG proteins in cancer. In addition, the cancer-specific Sat II RNA signature, and related CAP and CAST bodies, provide new candidate biomarkers of “heterochromatic instability”, and provide insight into the broader impact of epigenetic chemotherapeutics on the epigenome of normal and cancer cells. Each of these three areas of major contribution, for cancer epigenomics, satellite II biology, and biomarker discovery will be further discussed below.

Nuclear Re-Distribution of Chromatin Regulators and Epigenetic Imbalance in Cancer:

Tumor suppressor (TS) gene silencing paradoxically often co-occurs with the more global loss of repressive chromatin marks, particularly on repeats throughout the genome (Fraga et al., 2005). The grossly imbalanced nuclear distribution of master epigenetic regulators shown here, including polycomb proteins (PRC1) and methyl-binding proteins (MeCP2), provides a new way to think about how this epigenomic imbalance evolves in cancer cells. In a sense, visualization of these key regulatory factors and Sat II DNA/RNA provides a low resolution but “whole genome” synoptic view of their changed nuclear distribution and expression patterns, which may be less apparent by extraction-based analyses, particularly if repeats are excluded or if the protein levels are normal and believed to be unaltered.

Since mammalian PcG bodies have been studied almost exclusively using cell lines with tumor origins(Hernandez-Munoz et al., 2005; Saurin et al., 1998), our conclusion that prominent PcG bodies are aberrations of cancer is not inconsistent with prior studies (Voncken et al., 1999). Our results demonstrate that cancer nuclei commonly aggregate PcG proteins on particular Sat II domains that remain silent, while other Sat II loci in regions relatively depleted of these repressive factors now aberrantly express RNA. The fact that Sat II RNA mis-regulation is locus specific and co-occurs with, and is inversely related to, the marked redistribution of BMI-1 (and other PRC1 proteins) provides evidence for a functional relationship between the aberrant nuclear compartmentalization of regulatory factors and changes in locus-specific expression in cancer cells. Studies in Drosophila embryos demonstrate that access of specific genes to concentrated accumulations of PcG proteins is important to their regulation (Bantignies et al., 2011; Grimaud et al., 2006), supporting the importance of our findings that some regions of the cancer nuclear genome have dramatically higher access to PcG proteins than others. Our results predict that some regions of the cancer genome will contain hot spots of repression, whereas other regions will show wide scale reduction in repression, consistent with the loss of the silent peripheral heterochromatic compartment (Pageau et al., 2007). We demonstrate that this relates to locus-specific misregulation of Sat II loci, but it also could play a role in TS gene silencing or oncogene upregulation. Our findings would predict that many aberrantly expressed loci may be BMI-1 regulated, such as stem cell and neuronal genes (as well as Sat II loci). Importantly, our results further show that the abnormal satellite RNA accumulations have impact on the distribution of MeCP2 (and possibly other epigenetic factors), which we suggest likely further contributes to a downward spiral of the cancer methylome, and epigenomic imbalance.

Additionally, our results demonstrate an important new finding that link the nuclear distribution of these key cellular regulatory proteins of the PRC-1 complex to the vast literature on DNA methylation changes in cancer, particularly at the Sat II locus on 1q12. As further discussed below, the fact that chemotherapeutic demethylating agents rapidly induce PRC1 capping of 1q12 in normal cells is consistent with reports that Sat II at 1q12 is especially sensitive to de-methylation, and suggests that this may reflect an early event in the evolution of the cancer epigenome. Interestingly, the demethylation at 1q12 does not result in its expression when bound with PRC1 complexes, instead nuclear repeat RNA foci subsequently emanate from other de-repressed Sat II loci. Thus, it is the presence of the repressive PRC1 CAP bodies that rescues the affects of demethylation at this region. Notably, cellular demethylation through the use of 5-aza-2d is assumed to be responsible for the aberrant expression of numerous genes across the nucleoplasm in treated cells (Fabiani et al., 2010); however, the fact that Polycomb target genes are overrepresented in this group suggests that the redistribution of PcGs to CAP bodies may play a major significant role. Thus, methylation changes that result in the failed nuclear compartmentalization of repressive factors can promote broad heterochromatic instability (including further methylation changes); this in turn would generate an array of diverse expression profiles, any one of which might be selected for if it promoted neoplastic cell growth (Pageau et al., 2007).

Implications for the Biology of Human Satellite II:

Study of the abundant Sat II repeats in all human genomes has lagged far behind the rest of the genome; however, lack of known function is not evidence for no function. This study now implicates this repeat family as both reflecting and contributing to the epigenomic imbalance in cancer. Work presented here suggests new avenues of investigation for the potential biological import of Sat II DNA (and RNAs, below), based on the capacity of high copy simple repeats to underlie abnormal compartmentalization and sequestration of chromatin regulatory factors. This is most apparent for the very large pericentromere at 1q12, which is a universal but unexplained component of all human genomes. Theoretically, if each 26 bp Sat 2 repeat in two ˜6 Mb 1q12 loci could bind BMI-1 or a PRC1 complex, this locus alone could corral roughly 5×10⁵ such factors. Interestingly, BMI-1 proteins within PcG bodies have been shown to have low mobility (Hernandez-Munoz et al., 2005); since that study used U2OS osteosarcoma cells, our interpretation is that in cancer BMI-1 accumulates stably on 1q12.

Why PRC1 factors “pile up” on particular Sat II loci (primarily 1q12) in cancer nuclei remains an open question, but our results clearly link this to cytosine demethylation, which is the “switch” that promotes abnormal PRC1 binding to repeats across this huge locus. Results further suggest that this likely involves a distinct Sat 2 sequence sub-type at these loci, which BMI-1 may preferentially bind when demethylated. It is possible that the 1q12 locus undergoes similar changes during early development linked to some role in nuclear remodeling, since Sat II hypomethylation is reported in extra embryonic tissue (Zagradisnik and Kokalj-Vokac, 2000), although this remains speculative. Several earlier studies pointed out that 1q12 changes (breaks, amplications and gains of 1q) are unusually prominent in many cancers, with 1q gains in breast carcinoma long noted as particularly striking (Mertens et al., 1997). The findings here provide a clear path for further studies to understand how Sat II and DNA methylation changes relate to the abnormal compartmentalization of epigenetic factors shown here.

Sat II RNA, MeCP2, and the Concept of “Toxic Repeat RNAs”:

Another surprising aspect of our findings is the accumulations of MeCP2, which were clearly coincident with Sat II RNA foci in cancer cells. Of dozens of RNAs studied in our lab, such precisely overlapping RNA/protein signals (with same size and shape) were seen previously only for mutant CUG repeat RNAs, which we confirmed sequester MBNL1 in Myotonic Dystrophy (DM1) (reviewed in Osborne and Thornton, 2006; Smith et al., 2007), and NEAT I RNA which we showed is the structural scaffold for paraspeckle proteins (Clemson et al., 2009). Thus, this precise co-localization of an RNA and protein is significant, and suggest that they interact in some way. As noted above, these findings lend support for other evidence that MeCP2 can bind RNA (Hite et al., 2009), and acknowledge that the role(s) of methyl-binding proteins are not well explained by existing paradigms (Joulie et al., 2010). Another implication is that the satellite RNAs may themselves be cytosine methylated, as is known to occur for tRNAs and rRNA (Motorin et al., 2010). Since cytosine methylation can increase RNA stability, we note that aspects of our results hint that the accumulated Sat II transcripts are likely quite stable.

The accumulation of MeCP2 with Sat II RNA can be so marked in some tumor samples that just one or a few prominent “CAST” bodies are present in an otherwise dark nucleoplasm. As mentioned above, this suggests that these abundant repeat transcripts are not merely inert bi-products of epigenetic dysregulation, but can also impact the distribution of cellular factors and possibly contribute to further epigenetic imbalance. The potential for repeat RNAs to impact the distribution and availability of nuclear regulatory factors, and thereby impact expression of other genes, has strong precedence based on toxic repeat RNAs in certain triplet repeat diseases (Kanadia et al., 2003). Nuclear RNA accumulations of DMPK mRNA containing expanded CUG repeats sequester MBNL1, an alternative splicing factor, causing inappropriate splicing patterns that generate the Myotonic Dystrophy (DM1) phenotype (Osborne and Thornton, 2006). While neither Sat II RNA foci nor PcG bodies co-localize with MBNL1 or the “PNC compartment” linked to breast cancer (Kamath et al., 2005) (FIG. 13K), the paradigm that nuclear accumulations of “toxic repeat RNAs” can cause disease due to sequestration of regulatory factors that bind those repeats has been firmly established.

It is interesting to consider that Sat II RNA may also have a normal role during some developmental or cell cycle stage, which we think plausible despite the negative or negligible levels in normal cycling cells. For example, repeat RNAs may be involved in maintaining heterochromatin structure (Probst and Almouzni, 2007) and our results suggest, for example, that Sat II transcripts could recruit methyl-binding proteins.

Potential New Biomarkers Indicative of Heterochromatin Instability in Single Cells:

Finally, this study provides evidence for new epigenetic biomarkers in cancer, each visible in as little as a single cell in pathology sections of primary tumors. Sat II RNA is particularly attractive as a biomarker because it is essentially negative in normal cells, making this a sensitive assay that would also be amenable to extraction-based methodologies. While more extensive studies of tumor samples will be required, the case for Sat II RNA as a candidate biomarker is strengthened by a wholly independent study (Ting et al., 2011). Using deep sequencing, Ting et al investigated over-expression of repeat RNAs, and found Satellite II most clearly different from normal, in ten pancreatic cancers and in a few other tumor samples. Although neither study examined a large tumor sample, both came to similar conclusions about Sat II RNA over-expression using completely different approaches and tumor types, and found similar levels of Sat II up-regulation (130 fold in Ting et al. and 175 fold here). While we strongly detect satellite over-expression in most human cancer lines in vitro, Ting et al. concluded that this RNA was not over-expressed in cultured cancer cells (in three mouse tumor lines examined). This may either reflect a species difference or greater sensitivity of the fluorescence in situ assay. However, our study extends well beyond the initial discovery of satellite over-expression to investigate the basic biology behind it, leading to several novel and fundamental insights regarding nuclear compartmentalization and the imbalanced cancer genome. Ting et al. speculate that general de-repression of genomic repeats could arise by some common mechanism, but state that the concomitant “upregulation of diverse mRNAs is less readily explained” (Ting et al., 2011). Our findings not only provide an explanation for what we show is locus-specific de-repression of Sat II loci, but potentially why there would be broader de-repression of mRNA encoding genes, involving the sequestration of BMI-1 and MeCP2 on some genomic sites, at the expense of others. In support of this concept, we note that Ting et al. report that the mRNAs over-expressed were predominantly neuronal (which BMI-1 has been strongly linked to). In addition, inappropriate expression of neuroendocrine markers is common in many epithelial cancers and linked to aggressiveness (Cindolo et al., 2007).

Thus, Sat II RNA, CAP bodies, and CAST bodies are all potential “red flags” for major epigenetic dysregulation in cancer, which may prove to be a poor prognostic indicator. Cytopathological changes in nuclear and heterochromatin morphology are important diagnostic indicators of many cancers (Fischer et al., 2010), however the distinctions can be subtle and difficult to accurately identify. An advantage of the biomarkers and approach shown here is the potential to directly correlate these specific molecular signatures with the cytological diagnostic structural changes upon which the pathologist relies. In addition, our findings that the 5-aza 2′deoxycytidine (decitabine) can induce prominent BMI-1 bodies on 1q12, is revealing not only mechanistically but in terms of the often high toxicity of this drug (Gore et al., 2006), which likely will have unintended consequences on satellite and other heterochromatin (Jones and Baylin, 2007).

In conclusion, this study highlights that epigenomic changes will be more fully understood if the cancer genome is considered as a complex three dimensional entity within a highly sub-compartmentalized nuclear structure. As illustrated here, it will be necessary to examine DNA, RNA, and protein in precise relation within nuclear structure to uncover potentially key aspects of cancer biology. While many questions remain, these findings provide a foundation for new avenues of research bridging cancer epigenetics, nuclear structure, and the novel biology of DNA and RNAs from the repeat genome.

### Experimental Procedures:

Cell Lines, Growth Conditions & Fixation:

Twenty two cell lines were examined in this study (list in Supplement), and grown in conditions recommended by suppliers (ATCC, Cambrex, and Coriell). 5-azacytidine (6 mM) and 5-aza-2′deoxycytidine (0.2 ug/ml) was added fresh daily to asynchronously growing cultures and refreshed every day. Our standard fixation protocols have been detailed previously (Johnson et al., 1991; Tam et al., 2002), and summarized in the Supplement. Human effusions were fixed as for cultured cells and tissue blocks were cryosectioned onto cold glass slides (HistoBond+), and stored at −80 briefly until fixation. Of four fixations tested (Supplement) the one that gave best results was brief triton extraction followed by paraformaldehyde fixation and storage in ETOH.

FISH and IF:

Probes: L1 ORF2 (gift from J. Moran), XIST pG1A (from H. Willard & C.

Brown), and human Cot-1 DNA (Roche). Information on the Sat 2 probes used (Sat2-24 nt oligo, Sat2-59 nt oligo, Sat2-169 bp, & puc 1.77 kb), as well as Sat3 & HuAlphaSat (59 nt & 33 nt) oligos, is provided below.

Hybridization:

Sat2-24 nt LNA was used for most images unless otherwise indicated. Several methods of Sat 2 probe labeling and detection were tested (see below). RNA-specific hybridization was carried out under non-denaturing conditions where the DNA was not accessible. Oligos were usually hybridized at 15% formamide conditions, but were also compared to higher stringency hybridizations at 40% and 50% formamide.

Antibodies:

BMI-1 (from Dr. David Weaver, Upstate & Abcam), Ring 1B and EZH2 (Active Motif), MeCP2 and PTBP1 (Abcam), and MBNL (from Dr. Charles Thorton).

Microscopy and Quantitative Digital Imaging:

Digital imaging was performed using an Axiovert 200 or an Axiophot Zeiss microscope equipped with a 100× PlanApo objective (NA 1.4) and Chroma 83000 multi-bandpass dichroic and emission filter sets (Brattleboro, Vt.), set up in a wheel to prevent optical shift. Images were captured with the Zeiss AxioVision software, and an Orca-ER camera (Hamamatsu, N.J.) or a Photometrics 200 series CCD camera. Digital imaging software (Metamorph) was used to quantify signals (see below for details). Where required, care was taken to eliminate any bleed-thru of Texas-red fluorescence into the fluorescein channel. Most experiments were carried out a minimum of 3 times, and scored by at least two independent investigators. All findings were easily visible by eye through the microscope (unless otherwise noted), and images were minimally enhanced for brightness and contrast in Photoshop for publication (unless otherwise noted).

### Supplemental Methods

Human Cell Lines:

1) HSMM: Skeletal Myoblasts (Cambrex)

2) SUM 149PT: Inflammatory Breast Cancer (Asterand)

3) TIG-1: Fetal Lung Fibroblast (Coriell)

4) HCC1937: Breast Ductal Carcinoma (ATCC)

5) HCT: Colon Adenocarcinoma (ATCC)

6) HeLa: Cervical Adenocarcinoma (ATCC)

7) Hep-G2: Hepatocellular carcinoma (ATCC)

8) HFF: Foreskin Fibroblast (ATCC)

9) HT1080: Fibrosarcoma (ATCC)

10) IMR-90: Lung Fibroblast (ATCC)

11) JAR: Choriocarcinoma (ATCC)

12) MCF7: Breast Adenocarcinoma (ATCC)

13) MCF-10A: Breast Fibrocystic Disease (ATCC)

14) MDA-MB-231: Breast Adenocarcinoma (ATCC)

15) MDA-MB-436: Breast Adenocarcinoma (ATCC)

16) PC3: Prostate Adenocarcinoma (ATCC)

17) hTERT RPE-1: Telomerase immortalized retinal epithelial (ATCC)

18) SAOS-2: Osteosarcoma (ATCC)

19) T-47D: Breast Ductal Carcinoma (ATCC)

20) U2OS: Osteosarcoma (ATCC)

21) Wi38: Fetal Lung Fibroblast (ATCC)

22) WS-1: Embryonic Skin Fibroblast (ATCC)

Probe Sequences:

Sat 2 probes (Sat2-24 nt, Sat2-59 nt, Sat2-169 bp, & puc 1.77 kb) are distinct from one another (probes would not cross-hybridize), and appear to detect different “families” of Sat II. Sat II sequences contain degenerate forms of the 5 bp (ATTCC) Sat III motif, and consistent with this close relationship, the Sat 3 probe overlapped some Sat II RNA foci when used for RNA hybridizations (FIGS. 12A-12D); however the signal was reduced under higher stringency hybridization conditions (see below & Methods).

TABLE 1

| | | | | SEQ ID |
|---|---|---|---|---|
| Probe Name | Sequence | Label | Reference | NO. |
| Sat2-24nt LNA | 5'-ATTCCATTCAGA | 5' Biotin | Exiqon, Product # | 2 |
| (Exiqon) | TTCCATTCGATC-3' | | 200501-03 | |
| Sat2-24nt | 5'-ATTCCATTCAGA | 5' Alexa 488 | | 2 |
| (Invitrogen) | TTCCATTCGATC-3' | | | |
| Sat2-59nt | 5′-ANTCCATTCGGGTCC | 3′ Biotin or | Prosser et al., 1986 | 3 |
| Forward strand | ATTCGATGATGATCACACT | 5′FITC | | |
| (Invitrogen) | GGATTTCATTCCATAATTCT-3′ | | | |
| Sat2-59nt | 5′-CGAATAGAATTATGG | 5′FITC | | 4 |
| Reverse | AATGAAATCCAGTGTGATC | | | |
| compliment | ATCATCGAATGGACCCGAA | | | |
| (Invitrogen) | TGGANT-3′ | | | |
| Sat2-160bp | Fwd primer: Sat2 PCR F | Biotin PCR | Alexiadis et al., | 5 |
| | 5′-CATCGAATGGAAATG | label | 2007 | |
| | AAAGGAGTC-3′ | | | |
| | Rev primer: Sat2 PCR R-inv | | | 6 |
| | 5′-TTGACTGCAATCAT | | | |
| | CCAATGGT-3′ | | | |
| | Full Sequence Provided in | | | |
| | Appendix Below | | | |
| pUC1.77 (1q12) | 1.77kb, Partial sequence in | Biotin or | Cooke, 1979 | |
| | Cooke, 1979 | digoxigenin | | |
| | Full Sequence Provided in | nick translation | | |
| | Appendix Below | | | |
| Sat2_1q12 | Fwd primer: | (Full Sequence | | 7 |
| | 5′-GGAACCGAATGAATC | Provided in | | |
| | CTCATTGAATG-3′ | Appendix | | |
| | | Below) | | |
| | Rev primer: | | | 8 |
| | 5′-ATGATTCCATTCGATT | | | |
| | CAATGTTCCAT-3′ | | | |
| Sat2_7 | Fwd primer: | (Full Sequence | | 9 |
| | 5′ ATTCGATTCCATTCGA | Provided in | | |
| | TGATGATTCC-3′ | Appendix | | |
| | | Below) | | |
| | Rev primer: | | | 10 |
| | 5′-GGAACCGAATGAATC | | | |
| | CTCATTGAATG-3′ | | | |
| Sat2_16 | Full Sequence Provided in | | | |
| | Appendix Below | | | |
| Sat3 | 5′-CCATTCCATT | 3′Biotin | Prosser et al., 1986 | 11 |
| (Invitrogen) | CCATTCCATT-3′ | | | |

HuAlphaSat (59 mer) 5-‘CCT TTT GAT AGA GCA GTT TTG AAA CAC TCT TTT TGT AGA ATC TGC AAG TGG ATA TTT GG-3’ (Biosource & Invitrogen; SEQ ID NO: 12).
HuAlu (33 mer) 5′-CCC AAA GTG CTG GGA TTA CAG GCG TGA GCC ACC-3′ (Biosource; SEQ ID NO: 13).

Sat II probes can be used to detect different “families” of Sat II that show differential affinity for PcG proteins and for expression.

A highly sensitive 24 nt LNA oligo (Sat 2-24) was designed to maximize detection of Sat 2 family sequences. Hybridization to metaphase chromosomes with this LNA oligo detects Sat II loci on several chromosomes (including 1 and 16), consistent with a prior report (Silahtaroglu et al., 2004). This probe (under low stringency conditions) is also capable of detecting the more conserved Sat III locus on Chr 9. It also detects the highest number of expressed Sat II sequences in CAST bodies in cancer nuclei.

The 59 nt standard oligo to Sat II (Sat 2-59), described by (Prosser et al., 1986), detects Sat II of fewer chromosomes than Sat 2-24 (e.g. Chr 1, 16, 2, and 15), and none on the Sat III locus on Chr 9, and detects CAST bodies less robustly than Sat 2-24.

The PCR probe (Sat2_{—}7) detects a smaller subset of CAST bodies eminating from Chromosome 7 in some cancer samples, representing 4 different organ systems, suggesting that this locus may be susceptible to misregulation in a number of cancers.

Other Sat 2 probes (Sat2-160 bp, Sat2_{—}16, and puc 1.77 kb) have the most restricted distribution on Chrs. 1 and 16. These sequences correlate best with PcG distribution and do not detect appreciable RNA.

Because Sat II sequences are degenerate versions of the more conserved 5 bp Sat 3 sequence and often contain these sequences, the Sat 3 oligo (see table above), under low stringency, can also detect the same Sat II RNA foci as the Sat 2-24 LNA oligo.

Cell Fixation:

For our standard fixation conditions used in most experiments (Tam et al., 2002), cultured cells were grown on glass coverslips, and extracted in CSK buffer, 5% triton, and VRC (vanadyl ribonucleoside complex) for 1-3 min. Cells were fixed in 4% Paraformaldehyde for 10 min, then stored in 1×PBS or 70% ETOH. Four fixations were tested on frozen tissue sections: 1) our standard fixation protocol summarized above (this produced the best results), 2) Fixed first, extracted second, and stored in ETOH. 3) Fixed (4% Paraformaldehyde) for 10 min, no extraction, and stored in ETOH, and 4) 10 min incubation in PreservCyt (Cytic Corp) at rm temp and storage in ETOH.

RNA and DNA FISH & IF:

Our standard hybridization conditions for RNA, DNA, simultaneous DNA/RNA, and simultaneous DNA/IF or RNA/IF detection was performed as previously described (Johnson, Singer et al. 1991; Tam, Shopland et al. 2002), and briefly described below.

Oligo hybridizations were done overnight at 37 C, in 2×SSC, 1 U/ul RNasin and 15% formamide, with 5 pmol oligo or 0.1 pmol LNA oligo as indicated for lower stringency, or at 40-50% formamide for higher stringency.

Larger probe hybridizations were overnight at 37 C, in 2×SSC, 1 U/ul RNasin and 50% formamide, with 2.5 ug/ml of DNA probe. Cells were washed: 15% formamide/2×SSC at 37 C (20 min); 2×SSC at 37 C (20 min); 1×SSC at RT (20 min); and 4×SSC at RT (5 min).

Labeling and detection: Four methods of labeling and detection were used: 1) Larger (non-oligo) DNA probes were nick translated with biotin-11-dUTP or digoxigenin-16-dUTP (Roche Diagnostics, Indianapolis, Ind.), 2) the LNA oligo was end-labeled with either biotin or dig, 3), Sat2-59 nt was end-labeled with direct fluorochrome (Fite) or biotin, 4) and the PCR generated probe (Sat2-169 bp) used biotin. Detection utilized Alexa 488 or Alexa 549 Streptavidin (Invitrogen) in 1% BSA/4×SSC for 1 hr at 37 C. Postdetection washes: 4×SSC; 4×SSC with 0.1% Triton; and 4×SSC, each for 10 min at RT, in the dark.

For simultaneous RNA/DNA hybridizations, RNA hybridization was performed first (as above), fixed in 4% Paraformaldehyde for 10 min, then NaOH treatment, DNA denaturation and DNA hybridization. DNA was hybridized following denaturation. Briefly, the cells were treated with 0.2N NaOH in 70% ETOH for 5 min, rinsed with 70% ETOH then denatured in 70% formamide, 2×SSC, at 75 C for 2 min, before ethanol dehydration, and air-drying. Hybridization and detection was carried out as described above.

Simultaneous DNA/RNA and antibody detection: Most antibodies were used prior to RNA or DNA hybridization. Briefly, slides were incubated in the appropriate dilution of primary antibody in 1% BSA, 1xPBS and 1 U/ul RNasin, for 1 hour at 37 C. Slides were washed, and immunodetection was performed using 1:500 dilution of appropriately conjugated (Alexa 488 or Alexa 594, Invitrogen) secondary (anti-goat, mouse or rabbit) antibody, in 1×PBS with 1% BSA. The antibody signal is fixed in 4% paraformaldehyde for 10 min prior to hybridization (performed as detailed above), and all slides were counter stained with DAPI. Vectashield (Vector Labs) was used as mounting media for all fluorescence imaging.

Digital Quantification:

All images compared or quantified for signal intensity were taken with the same exposure on the same day with the same microscope and fluorochrome.

Linescans: The Linescan function in the Metamorph Image analysis software (Molecular Devices, Inc.) was used to measure relative signal intensities for each channel of a 3 color digital image of cell nuclei. Line regions were drawn across the entire nucleus of individual cells (unless otherwise noted) and pixel intensity along the line measured. Y-axis is intensity of each pixel across the length of the line (X-axis).

Maximum pixel intensity vs. threshold: Metamorph software was used to measure the single maximum pixel intensity of each cell nucleus. Three color images were used and the color channels separated. The regions outlining the nuclei on the DNA color channel were transferred to the channel containing the RNA signals. The single brightest pixel in each nuclear region was measured. This was then plotted against a threshold calculated for each cell line using 3× the average lowest intensity pixel in each nucleus for that cell line.

Total Sat RNA signal/cell: Metamorph software was used, and color channels separated for 3 color images. Computer generated regions were drawn around all RNA signals in each nucleus. The average pixel intensity for each region was multiplied by the area of each region, and then all regions in each nucleus were added to give the integrated intensity (area and brightness) for each nucleus.

TABLE 2
Aberrant Sat II foci present in most cancer cell lines and not normal cells.
Datasheets supplied with the cell lines report chromosome numbers for
T47D (~65), MCF-7 (~82), PC3 (~62) and U2OS (~70).

| | | | # of | Size of RNA |
|---|---|---|---|---|
| | | Aberrant | aberrant | Foci (microns) |
| | | Sat 2 | Foci Average | Average |
| Cell Line | Cell Type | RNA Foci | (Range) | (Range) |
| U2OS | Osteosarcoma | +++ | 6 (3-11) | 1.2 (0.9-1.6) |
| PC3 | Prostate Adenocarcinoma | ++ | 3 (1-6) | 0.9 (0.8-1.0) |
| MCF-7 | Breast Adenocarcinoma | ++ | 2 (1-4) | 0.98 (0.94-1.0) |
| HT-1080 | Fibrosarcoma | ++ | | |
| HCC-1937 | Breast Ductal Carcinoma | ++ | | |
| T47D | Breast Ductal Carcinoma | + | 2 (1-2) | 0.35 (0.25-0.56) |
| SAOS-2 | Osteosarcoma | + | 1 (1-2) | 0.2 (0.1-0.5) |
| HEP-G2 | Hepatocellular carcinoma | + | | |
| JAR | Choriocarcinoma | − | | |
| HELA | Cervical Adenocarcinoma | − | | |
| HCT | Colon Adenocarcinoma | − | | |
| MDA-MB-231 | Breast Adenocarcinoma | − | | |

Normal and non-cancerous cell lines

| | | |
|---|---|---|
| MCF-10A | Breast Fibrocystic Disease | − |
| IMR-90 | Lung Fibroblast | − |
| WS1 | Embryonic Skin Fibroblast | − |
| TIG-1 | Fetal Lung Fibroblast | − |
| HFF | Foreskin Fibroblast | − |
| HSMM | Skeletal Myoblasts | − |
| HSMM | Differentiated Myotubes | − |

TABLE 3
Fifteen out of thirty-seven human solid tumors are positive for aberrant
Sat II Foci, while none of the matched normals exhibit them.

| Tissue Bank | Patient | | | | Aberrant | Aberrant |
|---|---|---|---|---|---|---|
| Identifier | Age | Organ | Disease | Grade | Sat II RNA | Alpha Sat |
| 4386T | 33 | Breast | Adenocarcinoma | 3 | −/− | |
| 2597T*(−) | 46 | Breast | Carcinosarcoma | 3 | +/++ | −/− |
| 1403T | 30 | Breast | Ductal Carcinoma | 2 | +++/+++ | |
| 1533T | 71 | Breast | Ductal Carcinoma | 2 | −/− | −/− |
| 1659T | 57 | Breast | Ductal Carcinoma | 3 | +++/++ | +/+ |
| 1659N | 57 | Breast | Matched Normal | n/a | −/− | |
| 2205T | 37 | Breast | Ductal Carcinoma | 2 | −/− | −/− |
| 2334T | 53 | Breast | Ductal Carcinoma | 3 | +++/+++ | ++/+++ |
| 2334N | 53 | Breast | Matched Normal | n/a | −/− | −/− |
| 2356T | 48 | Breast | Ductal Carcinoma | 3 | +/++ | +/++ |
| 2389T | Unknown | Breast | Ductal Carcinoma | 1 | −/− | −/− |
| 4596T | 81 | Breast | Ductal Carcinoma | 2 | −/− | |
| 0934T | 85 | Breast | Ductal Carcinoma IS | 2 | −/− | −/− |
| 0934N | 85 | Breast | Matched Normal | n/a | −/− | −/− |
| 1404T*(−) | 30 | Breast | Lobular Carcinoma | 2 | −/− | −/− |
| 1645T | 67 | Breast | Lobular Carcinoma | n/a | −/− | −/− |
| 2175T | 56 | Breast | Lobular Carcinoma | 3 | −/− | −/− |
| 2175N | 56 | Breast | Matched Normal | n/a | −/− | −/− |
| 4267T | 47 | Breast | Lobular Carcinoma | 2 | −/− | −/− |
| 2004T | 71 | Breast | Metaplastic Carcinoma | 2 | ++/++ | −/− |
| 2734T*(−) | 46 Male | Breast | Metaplastic Carcinoma | 3 | ++/+ | |
| 0853T | 48 | Breast | Papillary Carcinoma | 3 | ++/+ | +/+++ |
| 0853N | 48 | Breast | Matched Normal | n/a | −/− | −/− |
| 2243N | 36 | Breast | Normal | n/a | −/− | |
| 2081T | 48 | Ovary | Adenocarcinoma | 3 | ++/++ | −/− |
| 2081N | 48 | Ovary | Not Malignant | n/a | −/− | −/− |
| 2142M*(+) | 50 | Ovary | Carcinoma (Metastatic) | 3 | ++++/++ | ++/+++ |
| 2980Ta | 75 | Brain | Glioblastoma | 4 | +/++++ | −/− |
| 2373T | 66 | Colon | Adenocarcinoma | 2 | −/− | +++/+++ |
| 1880T | 64 | Kidney | Renal Cell Carcinoma | 3 | +/+ | ++/++ |
| 1880N | 64 | Kidney | Matched Normal | n/a | −/− | −/− |
| 2311T | 66 | Lung | Squamous Cell | 2 | −/− | −/− |
| | | | Carcinoma | | | |
| 2312B | 87 | Pancreas | Serous Cystadenoma- | n/a | −/− | −/− |
| | | | Benign | | | |
| 2312N | 87 | Pancreas | Matched Normal | n/a | −/− | |
| 0520T Lt | 62 | Prostate | Adenocarcinoma | 7 (4 + 3) | −/− | −/− |
| 0540T | 62 | Prostate | Adenocarcinoma | 9 (4 + 5) | −/− | −/− |
| 0827T | 68 | Prostate | Adenocarcinoma | 7 (3 + 4) | −/− | −/− |
| 1630T RT | 57 | Prostate | Adenocarcinoma | 6 (3 + 3) | −/− | −/− |
| 1673T | 85 | Stomach | Adenocarcinoma | 2 | −/− | |
| 1673N | 85 | Stomach | Matched Normal | n/a | −/− | −/− |
| 2036T | 43 | Stomach | Adenocarcinoma | 3 | −/− | −/− |
| 2210T | 60 | Stomach | Adenocarcinoma | 3 | ++/++++ | |
| 2210N | 60 | Stomach | Matched Normal | n/a | −/− | −/− |
| 2233T | 47 | Stomach | Adenocarcinoma | 2 | −/− | −/− |
| 2539T | 68 | Stomach | GIST | 2 | +++/+ | −/− |
| 2539N | 68 | Stomach | Matched Normal | n/a | −/− | −/− |
| 2824T | 48 | Stomach | GIST | n/a | +/++ | |
| 2632T | 59 | Thyroid | Papillary Carcinoma | n/a | −/− | ++/+ |
| 2632N | 59 | Thyroid | Matched Normal | n/a | −/− | −/− |

TABLE 4
Presence of CAST and/or CAP Bodies
in Several Cancer Tissues Tested.

| Tissue Bank | | |
|---|---|---|
| Identifier | CAST | CAPS |
| 0853T | Yes | Yes |
| 0934N | No | No |
| 1403T | Yes | No |
| 1404T*(−) | No | Yes |
| 1533T | No | No |
| 1645T | No | Yes |
| 2004T | Yes | Yes |
| 2175T | No | No |
| 2334T | Yes | Yes |
| 2356T | Yes | Yes |
| 2597T*(−) | Yes | Yes |
| 2734T*(−) | Yes | Yes |
| 4267T | No | No |
| 4386T | No | No |
| 4596T | No | Yes |
| 2081T | Yes | Yes |
| 2142M*(+) | Yes | No |
| 2980Ta | Yes | No |
| 1880T | Yes | Yes |
| 2036T | No | Yes |
| 2210T | Yes | Yes |
| 2824T | Yes | No |

### Example 2

### Over-Expression of Satellite II RNA and Failed Nuclear Compartmentalization of Polycomb Proteins is Common in Human Breast Cancers and Provides a Sensitive Biomarker of Epigenetic Instability, Potentially Linked to Tumor Type, Stage or Aggressiveness

Human Pericentromeric Satellite II Repeats are Aberrantly and Grossly Expressed in Cancer:

Almost 50% of the human genome consists of repetitive sequence elements with high-copy tandem satellite repeats associated with centromeric regions, such as Satellite II, representing a major portion of the repeat fraction. While alpha-satellite (α-Sat) is at the centromere proper of all human chromosomes, Satellite II (Sat II) defines the pericentromere of several chromosomes, the largest (˜6 Mb) on Chr 1q12 and also Chr 16, and smaller Sat II on several other chromosomes. Sat II is comprised of thousands of ˜25 bp repeats, evolved from the 5 bp more conserved Sat III repeat on Chr. 9 (Richard et al. 2008). While long thought to be silent and have no known function (reviewed in Richard et al. 2007, Plohl et al. 2008), in yeast centromeric satellite siRNAs are implicated in heterochromatin maintenance (Volpe et al. 2002), although it is not clear these findings apply to mammalian satellites (reviewed in Probst et al 2007). We have discovered that in many cancer cells there is over-expression of “COT-1” RNA, which represents the broad repetitive fraction. After a comprehensive analysis of numerous repeat types, including SINES, LINES, alpha-Sat, Sat III, and Sat II, we discovered that grossly aberrant Sat II RNA expression is linked to cancer. Importantly, this robust Sat II expression is negative or negligible in normal cells, suggesting a highly sensitive and potentially specific marker. Moreover, it is readily visualized in single cells in a pathology section, indicating this assay can be both qualitative as well as quantitative.

Polycomb Proteins and Satellite Heterochromatin:

More recently we have uncovered an exciting connection between Sat II mis-regulation and the exceptionally important polycomb group (PcG) proteins which control much of the epigenome and are intensely studied for their strong links to cancer. PcG proteins induce repressive chromatin modifications on heterochromatin, thereby controlling most key developmental pathways in ES cells and embryos (Lee et al. 2006; Muyrers-Chen et al. 2004). BMI-1 is a key component of the PRC1 complex necessary for self-renewal of stem cells and suppression of the tumor suppressor locus Ink4a/Arf in stem cells and cancer (O'Carroll et al. 2001; Valk-Lingbeek et al. 2004). While over-expression of BMI-1 has been described in several cancers including breast (Pietersen et al. 2008), colorectal, liver, and lung (reviewed in Valk-Lingbeek et al. 2004) other results find its down-regulation is a poor prognostic indicator in breast cancer; thus its role in cancer progression and prognosis is currently unresolved but intensively studied (Glinsky et al. 2005; Pietersen et al. 2008).

We have discovered in cancer gross perturbation in the nuclear organization of PcG proteins (e.g., one or more of BMI-1, RING 1B, Phc1, Phc2, CBX4, CBX8, RNF2, SUZ12, EED, RBBP4, JARID2, EZH2, EZH1, RBBP7, GLI1, MYC, CDKN2A, and HST2H2AC) into prominent “Cancer-Associated Polycomb” (CAP) bodies. These CAP bodies form on the large 1q12 Sat II locus which remains silent, whereas PcG proteins are sequestered from the rest of the nucleoplasm, where other loci are inappropriately expressed.

Satellite RNA Misregulation is a Hallmark of Epigenomic and Heterochromatic Instability in Cancer:

Inappropriate expression of satellite repeat RNAs, coupled with aggregation of polycomb heterochromatin regulators into abnormal bodies, is an indicator of “heterochromatic instability”, which may be more common in cancers than realized, and has unexplored but important implications for cancer etiology, and potentially diagnostics. Given that this involves defective centromere associated heterochromatin, it has implications for chromosome segregation and for genetic as well as epigenetic instability. And while satellite over-expression may arise during cancer progression, it is likely linked to abnormal mitosis and epigenetic regulation and thus may contribute to progression.

Bioinarkers and Breast Cancer:

An important challenge in cancer medicine is to identify specific changes that occur in neoplastic progression, which may be common to many cancers, specific to particular types, or indicators of progression level (grade), aggressiveness or response to therapy. This will be vital for surveillance, recognition and proper classification of different cancer sub-types and for designing/evaluating therapeutic interventions. The cancer biomarkers described herein are “red flags” for major aberrations in epigenetic state, increasingly recognized as important to cancer progression and aggressiveness. The Sat II RNA promises high sensitivity, assayable in pathology tissue or extraction based methods, including potentially in blood or other bodily fluids, which would be extremely valuable. While cytopathological changes in nuclear morphology are important diagnostic indicators of many cancers, the distinctions can be subtle and would benefit from biomarkers that confirm cancer cell diagnosis in as little as a single cell. While the PcG protein sequestration requires immunohistochemical analysis, the Sat II RNA assay can be done rapidly on tissue with LNA oligos, or RT-PCR or microarray of lysates or blood.

A biomarker may be useful if it enhances detection of many cancers, or if it discriminates certain cancer sub-types or grades, or correlates with response to therapy. For example, in breast cancer there is a strong need for more biomarkers (Hinestrosa et al., 2007) to determine which in situ cancers or occult metastases are more prone to invasive progression. Improved biomarkers have potential to spare some patients unnecessary treatments and discriminate those who require more aggressive therapies. In fact, these may constitute “red flags” for a category of more “epigenetic cancers”, in which failed maintenance of chromatin state (defective chromatin remodeling) is particularly prominent or an early contributor to cancer development. As a biomarker, epigenetic instability has important implications for treatment, given the availability of newer pharmacologic agents that modulate histone modifications or DNA methylation state, and many have unintended impact on pericentric satellite heterochromatin. Compared to chromosomal instability, epigenetic alterations are also theoretically reversible.

Bridging Molecular and Cellular Information:

Studies on epigenetic components in cancer usually employ molecular analyses of extracted tissues, such as DNA methylation. Sat II RNA expression can be studied by, e.g., RT-PCR, while FISH and PcG (BMI-1 antibody) assays can be used to provide the advantage of epigenetic markers overlayed with key tissue and cell context for the pathologist.

As illustrated in FIGS. 16A and 16B and detailed in the Example 1 above, cancer cells in a breast carcinoma contain bright Sat II RNA foci (red) while the normal cells surrounding it do not (lower right). Quantitative microfluorimetry indicates Sat II signal is >175 fold above normal background fluorescence, in good agreement with recent findings from RNA sequencing analysis in pancreatic cancer (Ting et al., 2011). This Sat II RNA comprises a major portion of total RNA and is assayable by both in situ and extraction based methods. Moreover, we have also discovered (Example 1 above) a compelling link between aberrant nuclear bodies of polycomb (PcG) proteins (e.g., one or more of the PcG proteins described herein as being associated with CAP bodies, in particular BMI-1 and Ring1B) and Sat II RNA in cancer. These cancer-associated PcG bodies reflect highly abnormal compartmentalization of key regulatory factors highly concentrated at some genomic repeat regions but depleted from much of the nucleoplasm.

We have discovered that Sat II RNA can be used as a biomarker to provide a “black and white” difference between normal cells and cancer cells. Our results in cell lines and a limited sample of tumors suggest a high incidence of Sat II RNA expression in breast cancer, which impacts 1 in 9 women (Tables 5 and 6). Both RT-PCR and molecular cytology, as well as other RNA biomarker assays (see, e.g., Tafe et al., 2010), can be used to assay the presence of Sat II RNA, which is expected to provide higher sensitivity than other biomarkers, in a panel of breast cancer sentinel lymph nodes (SLN) and other available well characterized tumors. Sat II RNA can also be detected in other bodily fluids, such as blood, using approaches similar to those currently pursued for microRNAs (see, e.g., Gao et al., 2011), which tend to have much less marked expression differences compared to Sat II RNA.

Sat II RNA Expression and CAP Bodies as Biomarkers in a Panel of Primary Breast Tumor Samples of Different Types and Grades.

Sat II RNA and CAP bodies are epigenetic “signatures” that can be used as robust cytological biomarkers of particular sub-types or stages of breast cancer, and these biomarkers can be used for cancer diagnosis and prognosis. Results in cell lines and several tumor samples predict Sat II RNA expression (and PcG bodies) will be seen in many breast tumors.

Sat II RNA Expression Detection by RT-PCR in a Panel of 59 Breast Cancer Sentinel Lymph Nodes.

Sat II RNA as a biomarker for breast cancer detection can be confirmed by using RT-PCR in already available lysates for comparison as a biomarker of occult metastasis and/or poor prognostic indicator. Analysis of pathology sections of nodes could also be used to determine if micrometastasis differ in expression of “epigenetic biomarkers” and whether this links to known survival and clinical pathology data.

Satellite II is Very Commonly Aberrantly Expressed in Cancer Lines and is Absent or Negligible in Normal Cells.

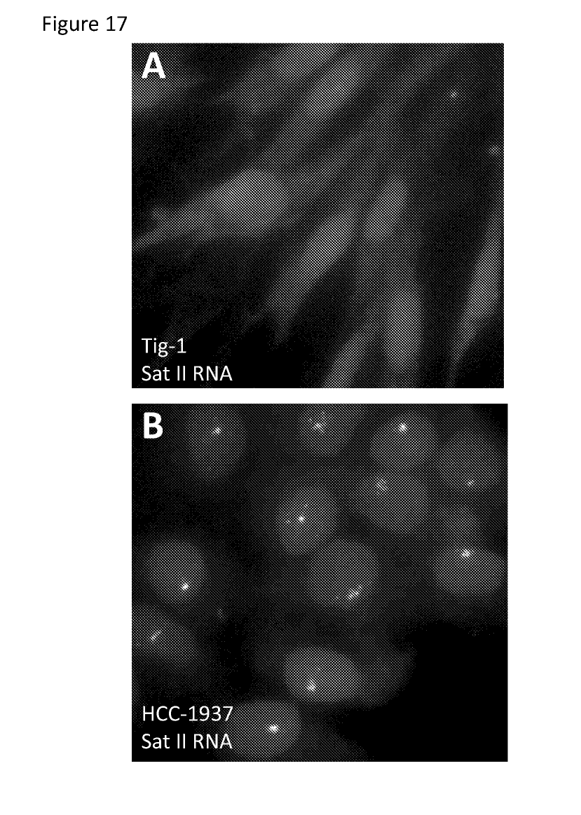

Use of a number of oligonucleotide probes for Sat II has revealed that prominent, aberrant foci of Sat II RNA are seen in eight of twelve cancer cell lines, whereas Sat II RNA is absent or negligible in all six normal somatic cell lines (Table 5). The clear difference between cancer and normal cells was very distinct (FIGS. 17A and 17B). Not only was it visible easily by eye through the microscope (scored by four independent investigators), but was obvious at low magnification (as used for pathology slides) and was easily confirmed by several methods of quantitative digital microfluorimetry (e.g., FIG. 2E), some of which may be amenable to automation.

TABLE 5
Eight of the twelve cancer lines examined showed over-
expression of satellite II RNA, and none of the normal.

| | | Aberrant |
|---|---|---|
| | | Sat II |
| Cell Line | Cell Type | RNA Foci |
| U2OS | Osteosarcoma | +++ |
| PC3 | Prostate | ++ |
| | Adenocarcinoma | |
| MCF-7 | Breast Adenocarcinoma | ++ |
| HT-1080 | Fibrosarcoma | ++ |
| HCC-1937 | Breast Ductal Carcinoma | ++ |
| T47D | Breast Ductal Carcinoma | + |
| SAOS-2 | Osteosarcoma | + |
| HEP-G2 | Hepatocellular carcinoma | + |
| JAR | Choriocarcinoma | − |
| HELA | Cervical | − |
| | Adenocarcinoma | |
| HCT | Colon Adenocarcinoma | − |
| MDA-MB-231 | Breast Adenocarcinoma | − |
| −−−−−−−−−−−−−−−−−−−− | −−−−−−−−−−−−−−−−−−−−−−−−−−−−−−− | −−−−−−−−−−−−−−− |
| *MCF-10A | Breast Fibrocystic | − |
| | Disease | |
| *IMR-90 | Lung Fibroblast | − |
| *WS1 | Embryonic Skin | − |
| | Fibroblast | |
| *TIG-1 | Fetal Lung Fibroblast | − |
| *HFF | Foreskin Fibroblast | − |
| *HSMM | Skeletal Myoblasts | − |
| *HSMM | Differentiated Myotubes | − |

Accumulations of Polycomb Proteins into Polycomb “Bodies” (PcG Bodies) is not a Feature of Normal Cells, but are Only Commonly Seen in Cancer Cells.

We find PcG bodies are almost exclusively found in cancer cells (7 out of 8 cancer lines were positive) and not normal cells (none of 5 non-neoplastic lines examined). Thus, we believe that the presence of PcG bodies is a hallmark of human cancer cells and are not structures of normal nuclei.

PcG Bodies are Associated with the Large Accumulations of Sat II DNA on Chromosomes 1, Which are not Expressing RNA.

PcG bodies form on the huge Sat II block on Chr 1q12 which remains transcriptionally silent. We find that PcG bodies and Sat II RNA appear to be mutually exclusive. Thus, Sat II RNA appears to be expressed only from loci that are not associated with accumulations of repressive PcG proteins. (Rather, PcG proteins may be sequestered away from loci that now inappropriately express Sat II.)

Aberrant Sat II RNA Foci and PcG Bodies are Also Observed in Solid Human Tumor Tissue and Not Normal Tissue.

Although aberrant satellite RNA and PcG bodies are not found in cultured normal cells, suggesting they did not arise as a consequence of cell culture, the question remained whether these foci can be seen in vivo (human tumors). We have also examined Sat II RNA over-expression and PcG protein distribution in frozen sections of 6 tumors from the Umass Tissue Bank and some of their matched normals. After working out proper fixation protocols that adequately preserved poly-A RNA (our positive control), we found that both PcG bodies and aberrant Sat II foci are commonly seen in human tumor tissue sections (5 of 6 tumors were positive) and not in matched normal tissue sections (FIGS. 9A and 9B and Table 6) or in normal cells in the tumor section.

TABLE 6
Aberrant Sat II foci in frozen human
tumor samples and matched normals.

| Tissue | | | | Differenti- | Sat II |
|---|---|---|---|---|---|
| Identifier | Organ | Disease | Grade | ation | RNA |
| 2334T | Breast | Ductal carcinoma | 3 | Poor | +++ |
| 2334N | Breast | Matched Normal | n/a | n/a | − |
| 2205T | Breast | Ductal carcinoma | 2 | Moderate | + |
| 1659T | Breast | Ductal carcinoma | 3 | Poor | ++ |
| 1880T | Kidney | Renal cell | 3 | ND | + |
| | | carcinoma | | | |
| 1880N | Kidney | Matched Normal | n/a | n/a | − |
| 2081T | Ovary | Cancer | 3 | Poor | +++ |
| 2312T | Pancreas | Serous cystadenoma | benign | n/a | − |
| 2312N | Pancreas | Matched Normal | n/a | n/a | − |

Evidence of Sequestration of PcG Proteins from the Rest of the Nucleus.

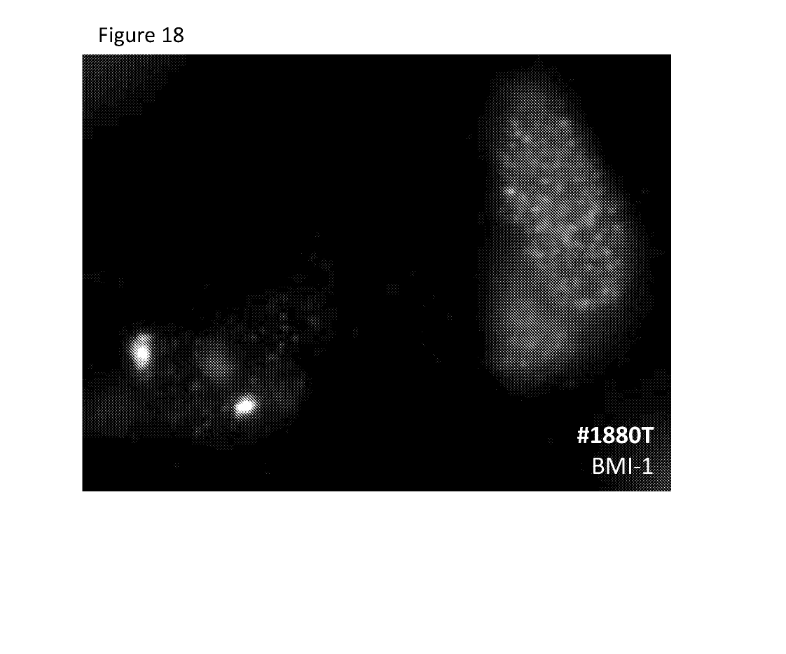

The presence of one or more prominent PcG bodies was often accompanied by marked sequestration of BMI-1 from the rest of the nucleoplasm (FIG. 18) including regions now expressing Sat II. Thus, the co-occurrence with PcG bodies further substantiates the link between abnormal PcG distribution and aberrant Sat II expression. Finally, this suggests that aberrant Sat II expression likely occurs via the sequestration or failed compartmentalization of the master developmental regulators of heterochromatin formation, polycomb proteins.

We have demonstrated that Sat II RNA is expressed in cancer but not normal cells, and co-occurs with formation of aberrant cancer-associated PcG bodies. This was shown in numerous cancer cell lines as well as a small sample of primary tumors and ascites, including three breast ductal carcinomas and one ovarian tumor, all of which showed these hallmarks. We believe that Sat II RNA, as a biomarker of cancer, can be as a hallmarks to determine the sub-type, grade and/or clinical outcome (prognosis) of cancer (e.g., primary breast tumor). We also believe that Sat II RNA can be used as a sensitive indicator of metastatic cells in sentinel lymph nodes, and that Sat II expression can be used to correlate clinical outcome. Sat II RNA can also be assayed from a patient's bodily fluid to detect metastatic disease.

Sat II RNA is negative in normal cells and thus can be used as a highly sensitive indicator for the presence of at least some types of cancers (e.g., breast cancer and pancreatic cancer), assayable by a number of methods. Very recently a study appeared in Science reporting over-expression of Sat II RNA in ten of ten pancreatic tumors examined and proposing it should be pursued as a potential biomarker (Ting et al., 2011). Our data show that Sat II RNA over-expression is linked to sequestration of essential epigenetic regulators (PcG proteins) into aberrant nuclear bodies, and thus both Sat II RNA and PcG bodies indicate major epigenetic dysregulation; the presence of one or both biomarkers in a cell of a patient likely indicates a poor prognosis.

Sat II Expression and CAP Bodies can be Used to Type and Grade Primary Breast Tumor Samples

The presence of Sat II RNA and PcG foci is common in many breast tumors and may be linked to cancer sub-type, aggressiveness, or grade. The prevalence of Sat II RNA over-expression and PcG mislocalization in a large number of primary breast tumors may be related to clinicopathologic data. As explained above, since Sat II and PcG bodies often co-occur and reinforce one another as indicators of epigenetic instability (FIGS. 16A and 16B) these can be analyzed together or in parallel. PCR analysis for Sat II RNA can be used, as well as molecular cytological analysis of cancer tissue sections to determine the extent of Sat II RNA and PcG body signatures in primary breast tumors of different types.

All UMass specimens are registered with the North American Assoc. of Central Cancer Registries (NAACCR), and NCI's SEER program and have long term clinical outcome data available. OCT blocks have about 5-10 years of outcome data, while the archival paraffin samples are longer. Although we will initially use frozen OCT specimens from The Tissue Bank, we will seek to expand this into archival paraffin specimens using antibodies to BMI-1 to mark PcG bodies and in situ hybridization to probes for Sat II RNA. Poly-A RNA hybridization will provide an internal control for RNA preservation in every sample.

The “epigenetic markers” described herein may be used to discriminate a specific known (or unknown) sub-type of breast cancer. Mis-regulation of Sat II and PcGs may be a feature of many or all types of breast cancer. Thus, the biomarkers described herein may be use to identify cancer sub-types and clinical/pathological parameters, including grade, lymph node and distant metastases (stage), ductal vs lobular type, the presence of lymphatic or vascular invasion, estrogen and progesterone receptor status, ploidy, growth fraction by Ki 67 immunostaining, Her2 status, BRCA1 mutation status, complete response to neo-adjuvant chemotherapy, and occurrence of triple negative and basal phenotypes.

The biomarkers identified herein may also be used for early tumor detection or to discriminate a progression-prone cancer. About 40% of samples available through the tissue bank will contain non-invasive carcinoma in situ and varying degrees of pre-cancerous hyperplastic changes, and we can ascertain the stage in the multistep process of breast cancer development at which Sat II RNA or PcG bodies develop. The Sat II RNA fluorescence signal can also be quantified by microfluorimetry, and show a good agreement with extraction based methodologies.

Statistical analysis: Differences between tumor categories can be evaluated by analysis of variance (ANOVA), and pairwise comparisons made using Tukey's HSD multiple comparisons procedure. The strength of correlation between the new biomarkers (Sat II RNA, CAP bodies, and CAST bodies) with each other and with the other clinically-significant descriptors of the tumor can be determined to assess relationships between biomarkers and clinical and pathologic variables, using Pearson product moment correlations for continuous normally distributed variables or Spearman's Rank Correlation Coefficient for non-normally distributed or rank order variables.

Primary tumor samples can be characterized for their Sat II RNA/CAST/CAP signatures, thereby identifying which primary tumor types exhibit these aberrant marks, similar to that performed for cancer cell lines and tumor samples (Tables 5 and 6). While initial scoring can be done through the microscope, quantitative digital microfluorimetry can also be used to quantify differences (e.g., FIG. 2E). For example, to be considered positive a sample might contain Sat II RNA foci intensity that is at least 3 fold above background levels. If the number of cells found positive in normal samples is essentially zero, then even 10% of positive cells in the tumor would have significance, although we would consider a strong positive to show RNA foci in 30-90% of cells, as seen in some of our cancer cell lines and tumor samples.

Sat II RNA can be Used as a Sensitive Detector or Prognostic Indicator of Metastases in Breast Sentinel Lymph Node by RT-PCR and Cytology and Initial Tests in Blood:

We have shown Sat II

RNA over-expression in primary breast tumors using in situ hybridization (FIGS. 16A and 16B and Tables 5 and 6). RT-PCR can also be used to assay for Sat II RNA. Primers described herein can be used in the RT-PCR assay to distinguish between known positive and negative cells and samples, and this technique can be applied to the analysis of lymph node samples to investigate detection sensitivity, and the results can be correlated to clinicopathologic data. RNA FISH assay can also be used to assay for Sat II RNA using, e.g., OCT preparations of the nodes.

SAT II RNA can be detected in breast sentinel lymph nodes via RT-PCR. Primers have already been made based on consensus sequences targeting all SAT II RNA elements as well as others specifically for the SAT II locus on Chr. 7, which analysis of available RNA sequence data indicates is particularly over-expressed. These primers can be used for specific detection of SAT II RNA, e.g., in U2OS osteosarcoma that highly express SAT II RNA relative to normal fibroblasts which show no expression. We will first do Trizol extractions of the RNA, treat the samples with RNase-free DNase, followed by RT-PCR with our SAT II primers with an RT-minus control, then visualize products by semi-quantitative gel electrophoresis. If initial results indicate a significant difference in expression levels of Sat II RNA, as predicted, we will perform quantitative Real Time RT-PCR. We will initially compare the U20S Sat II expression level with that of TIG-1 (fetal lung fibroblast) cells. Expression levels will be normalized to that of a housekeeping gene.

The primers can also be used to detect Sat II RNA in clinical samples, with emphasis on the 59 RNA lysates of breast sentinel lymph node biopsies. An appropriate normal mRNA can be included as a control for RNA preservation. The Sat II RNA assay can be used as a sensitive assay for the detection of micro-metastases.

The presence or absence of Sat II RNA in micro-metastases correlates with clinical outcome. We believe that whether a sub-type of breast tumor expresses or does not express Sat II RNA may correspond with aggressiveness. The absence of this hallmark of epigenetic instability may correlate with better outcome, e.g., if nodes known to contain metastatic cells differ with respect to whether they contain Sat II RNA.

SAT II RNA detection could be used for non-invasive testing. Currently, breast sentinel node biopsies are the standard for detecting invasive cancer, but clearly it would be enormously important if Sat II RNA could be detected in bodily fluids of women with metastatic or more localized disease. Because Sat II RNA appears to be unusually stable, possibly due to methylation, this biomarker could be used in a non-invasive assay to diagnose cancer. Current studies in various fields indicate the presence of cell-free RNA in the blood, which can potentially be used diagnostically. To test this approach, RT-PCR can be performed on U2OS cell culture media, and the presence of cell-free SAT II RNA can be detected in the filtered culture media. This approach could be used to test blood or lymph samples of women known who have breast tumors for the presence of SAT II RNA.

### Example 3

### DNA Hybridization with a Probe to the 1q12 Satellite II Locus to Assay for Aberrant Increase in Representation of this 1q12 Satellite in Cancer

All normal human cells have just two copies of the largest (6 Mb) satellite II locus on Chr 1q12, one on each of the two homologous chromosomes (illustrated in Example 1, FIG. 4A and FIG. 6D). Prior to our findings, this satellite II locus had no known function in normal cells or disease, but our findings show that it is the 1q12 satellite specifically that is involved in the mis-compartmentalization of polycomb proteins in cancer.

As shown in FIG. 4B (discussed in more detail in Example 1 above), cancer cells may be characterized by the presence of an increased number of this 1q12 satellite locus. Fluorescence in situ hybridization to cellular DNA using a cloned probe (puc 1.77 DNA) that specifically detects the 1q12 satellite locus clearly shows that in the nucleus of this U20S osteosarcoma cell there are three 1q12 satellite loci, instead of the normal two. FIG. 4C shows that each of these three 1q12 satellite loci specifically binds high concentrations of the polycomb group protein BMI-1 (and thus depletes this regulatory factor from the rest of the nucleoplasm). Therefore, DNA FISH or other methods to quantify 1q12 DNA in a cell may be used to examine aberrant copy numbers of this region in cancer, which in turn further promotes aberrant compartmentalization of polycomb group proteins. Similarly, other methods involving extraction of nuclear DNA followed by, e.g., Southern blot, PCR, or other sequence-determining methods can be used to quantify whether there are amplified levels of 1q12 satellite DNA in a sample. In addition, methods, such as bi-sulfite sequencing, can be used to determine not only the copy number but the methylation status of that 1q12 DNA.

As noted in Example 1, an earlier survey of chromosome aberrations in cancer (Mehrtens et al., 1997) noted that there is an unexplained correlation between increased copy number of the long arm of Chr 1q (over 100 Mb of DNA) and certain cancers, as was prominent in breast cancer. However, this finding was not useful diagnostically because such a broad and non-specific region of the largest human chromosome was examined, and it was unknown if any particular region of 1q might have an involvement in cancer. Our findings show for the first time that the 1q12 satellite locus is directly involved in the highly aberrant distribution of master epigenetic regulators in the cancer epigenome. Thus, either the formation of cancer-associated polycomb bodies (which form on 1q12) or the increased copy number of 1q12 satellite DNA can be assayed as an indicator of epigenetic dysregulation linked to cancer.

As shown in Example I, FIGS. 6A-6D, our findings further show that the aberrant compartmentalization of polycomb proteins, such as BMI-1, on 1q12 DNA is directly induced by DNA de-methylation of this large satellite locus. Studies focused primarily on DNA methylation changes of tumor suppressor genes have noted that 1q12 satellite DNA is very commonly demethylated in cancer, however this was not known to have a functional impact or significance for cancer progression. Our findings provide evidence that it is demethylation of 1q12 satellite II DNA that causes aberrant polycomb body formation, and thus show that the methylation status of 1q12 specifically contributes to broader epigenetic imbalance in the cancer nucleus.

### Example 4

### Epigenetic Imbalance in Cancer Cells Correlates with BRCA1 Deficiency

The BRCA1 protein contains a RING finger domain in the amino terminus with ubiquitin E3 ligase activity and two BRCT repeats in the carboxy terminus. BRCA1 is highly expressed in proliferative cells and its loss leads most prominently to genetic instability and growth arrest. BRCA1 is responsible for the monoubiquitylation of histone H2A and disruption in this process impairs the integrity of constitutive heterochromatin, which leads to a disruption of gene silencing at tandemly repeated DNA regions, in particular in regions containing satellite DNA.

Defects in BRCA1 increase the risk of cancer in patients, in particular breast and ovarian cancer. As is known, a diagnosis of cancer in a mammal (e.g., a human) can be made by detecting a mutation in a BRCA1 gene or in a BRCA1 protein that prevents the monoubiquitylation of histone H2A (see Zhu et al., Nature 477:179, 2011). Also, a diagnosis of cancer in a mammal can be made by detecting a decrease in the monoubiquitylation of histone H2A. Furthermore, mutations that prevent BRCA1 from ubiquitylating histone H2A produce an imbalance in the epigenome that results in an increase in the expression of satellite II RNA and the formation of CAP and CAST bodies. Thus, the methods of this application, such as the detection of an increase in the expression of satellite II RNA and detection of the formation of CAP and CAST bodies, can be performed in combination with the detection of mutations in a BRCA1 gene or in a BRCA1 protein or a detection of the decrease in the monoubiquitylation of histone H2A using a sample from a patient having, or at risk of, cancer.

In addition, in view of the role that mutations in a BRCA1 gene or in a BRCA1 protein that prevent the monoubiquitylation of histone H2A play in producing epigenetic imbalance, it is now possible to screen agents for their suitable in the treatment of a cancer in a mammal (e.g., a human) by contacting a cancer cell that includes a mutation in a BRCA1 gene or in a BRCA1 protein that prevents the monoubiquitylation of histone H2A, or a cell that exhibits a decrease in monoubiquitylated histone H2A, with the agent in order to determine whether the agent increases the monoubiquitylation of histone H2A in the cell. This assay can be performed as the sole assay or it can be performed by also determining the effect of the agent on other biomarkers, such satellite II RNA molecules and CAP and CAST bodies, in the cancer cell, as is discussed herein.

Finally, increases in epigenetic imbalances caused by a chemotherapeutic agent can also be determined by contacting a cell (e.g., a non-cancer cell) with the chemotherapeutic agent and determining the level of monoubiquitylation of histone H2A in the cell. A determination that the chemotherapeutic agent decreases the monoubiquitylation of histone H2A in the cell (i.e., causes an increase in epigenetic imbalance) indicates that the chemotherapeutic agent should not be administered for the treatment of cancer.

### Example 5

### Imbalance of UbH2A Distribution in Cancer Cells Correlates with Cancer

An imbalance in the distribution of UbH2A has also been correlated with a cancer genome. As shown in FIGS. 19A and 19B, a ChIP-Seq approach was used to detect a “patchy” distribution of UbH2A in osteosarcoma cells (FIG. 19A) relative to Tig-1 cells (normal fibroblasts; FIG. 19B). Thus, an imbalance in UbH2A in the genome of a cell is a further hallmark of epigenetic imbalance that can be used to detect the risk of cancer in a patient.

The distribution of UbH2A (as seen in FIG. 19A) can be quantified by analyzing the standard variation of UbH2A distribution across the genome (e.g., large areas of depletion and accumulation). The distribution of UbH2A is much higher in a cancer sample, relative to a normal sample, and shows a clearly statistically significant difference.

ChIP is a powerful method to selectively enrich for DNA sequences bound by a particular protein in living cells, in this case UbH2A. The ChIP process enriches specific crosslinked DNA-protein complexes using an antibody against a protein of interest. After size selection, all of the resulting ChIP-DNA fragments are sequenced simultaneously using a genome sequencer. A single sequencing run can scan for genome-wide associations with high resolution, meaning that features can be located precisely on the chromosomes.

Methods can also be used that analyze the sequences by using cluster amplification of adapter-ligated ChIP DNA fragments on a solid flow cell substrate to create clusters of approximately 1000 clonal copies each. The resulting high density array of template clusters on the flow cell surface can be sequenced by a Genome analyzing program. Each template cluster undergoes sequencing-by-synthesis in parallel using novel fluorescently labelled reversible terminator nucleotides. Templates are sequenced base-by-base during each read. Then, the data collection and analysis software aligns sample sequences to a known genomic sequence to identify the ChIP-DNA fragments.

Sensitivity of this technology depends on the depth of the sequencing run (i.e. the number of mapped sequence tags), the size of the genome and the distribution of the target factor. Unlike microarray-based ChIP methods, the precision of the ChIP-Seq assay is not limited by the spacing of predetermined probes. By integrating a large number of short reads, highly precise binding site localization is obtained. Compared to ChIP-chip, ChIP-Seq data can be used to locate the binding site within few tens of base pairs of the actual protein binding site. Tag densities at the binding sites are a good indicator of protein-DNA binding affinity, which makes it easier to quantify and compare binding affinities of a protein to different DNA sites.

Methods

ChIP-seq was performed as previously described (Yildirim et al., 2011) with some modification. Approximately 1×10⁶ cells were crosslinked with formaldehyde to a final concentration of 1% for 10 minutes at room temperature and stopped by the addition of 125 mM glycine. Cells were washed twice with 1×PBS containing protease inhibitors (Roche complete Mini protease inhibitor tablets) and pelleted at 100 rpm at 4° C. for 5 min. Cell pellets were resuspended in SDS lysis buffer (1% SDS, 10 mM EDTA, 50 mM Tris-Cl pH 8.1) with protease inhibitors and incubated on ice for 10 min. Cells were then sonicated at 10% duty, setting 2 for 10 minutes to a fragment size of 150-500 nt followed by centrifugation at 3000 rpm for 10 min at 4° C. Supernatant was collected and 100 uL chromatin was incubated with an antibody against Ubiquityl Histone H2A (UbH2A, Cell Signaling #8240) as per manufacturer's recommended concentrations at 4° C. overnight with rotation in IP Buffer (0.01% SDS, 1.1% Triton X-100, 1.2 mM EDTA, 16.7 mM Tris-Cl, pH 8.1, 167 mM NaCl)+0.5% BSA. 50 uL protein G magnetic beads (Cell Signaling, #9006) was added to antibody-chromatin complex for 4 hours at 4° C. with rotation. CUP washes were as follows: 2×IP Buffer, 2×RIPA buffer (0.1% SDS, 10 mM Tris, pH 7.6, 1 mM EDTA, 0.1% Na-deoxycholate, 1% Triton X-100), 2×RIPA buffer+0.3M NaCl., 1× LiCl Buffer (0.25M LiCl, 0.5% NP-40, 0.5% Na-deoxycholate), 1×TE. Crosslinks were reversed overnight at 65° C. in 1×TE with the addition of 3% SDS, 1 mg/mL proteinase K, 200 mM NaCl. DNA was extracted with phenol:chloroform and precipitated with 0.1× volume 3M NaOAc, pH 5.2 and 2.5× volume 100% EtOH overnight at −20° C.

Preparation of Illumina paired end deep sequencing ChIP libraries was performed as described (Yildirim et al., 2011). Deep sequencing data was mapped to human genome build hg19 using Bowtie (Langmead, Trapnell, Pop, & Salzberg, 2009). Data normalization and peak calling was performed over a 10 kb sliding window using SeqMonk (Babraham Bioinformatics, Babraham Institute, Cambridge, UK).

### Example 6

### Mutations in the BRCA1 Gene Strongly Re-Dispose to Breast and Ovarian Cancer

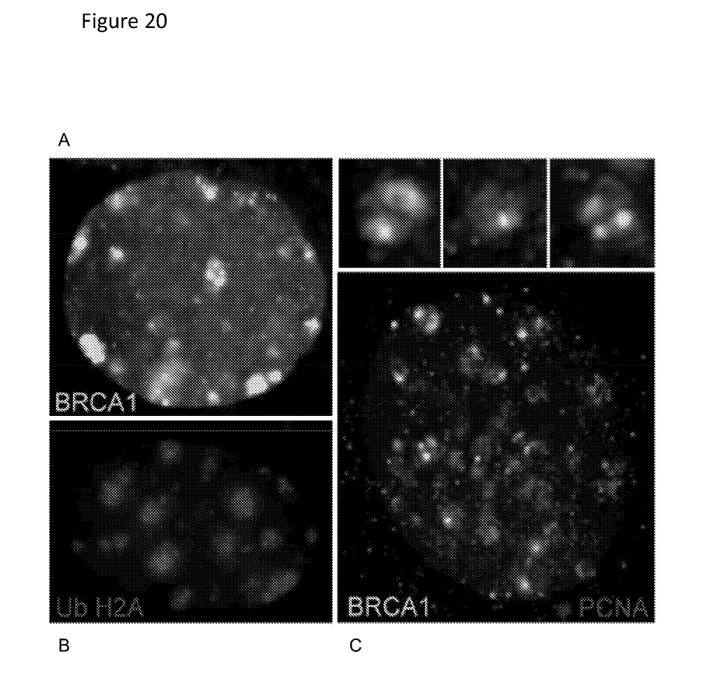

The BRCA1 tumor suppressor, a ubiquitin ligase, is implicated in multiple nuclear functions, including DNA repair and recombination. In irradiated nuclei, BRCA1 foci localize to sites of DNA repair with other repair proteins. While the link to DNA repair has been extensively studied, the potential role of BRCA1 foci in normal S-phase nuclei has been relatively ignored. The typical 5-15 foci consistently present in S-phase nuclei are widely presumed to be just storage sites or endogenous repair. However, these foci could actually reflect an undiscovered aspect of BRCA1 function; key to this question is whether they form at specific genomic sites. In the course of studying BRCA1 in relation to XIST RNA and X-inactivation, we recently discovered that many BRCA1 foci directly abut or overlap markers of the interphase centromere/kinetochore complex. Mouse nuclei have prominent chromocenters reflecting a defined organization of centric and pericentric heterochromatin; the association of BRCA1 foci with these can be striking, particularly in a subset of cells that label with PCNA, a replication marker (see FIG. 20A-20C). A recent study provided evidence showing that BRCA1 is involved in DNA decatenation in normal S-phase nuclei and that this is linked to the ubiquitination of topoisomerase II, however this study did not address the relationship to S-phase foci and whether BRCA1 may have a more localized function.

BRCA1 has a fundamental but previously unrecognized role in centromere structure and function; this in turn may impact chromosome segregation and maintenance of genomic stability. Our findings show that BRCA1 foci have a substantial though incomplete association with interphase centromere-linked structures.

BRCA1 functions routinely during S-phase. Rather than being required for segregation of sister chromatids, BRCA1's role may be more focused at centric or pericentromeric DNA, the highly repetitive nature of which may pose special requirements for decatenation and/or chromatin modification. The BRCA1 S-phase pattern does not simply mirror that of replicating DNA, but may reflect a subset of replicating DNA.

BRCA1 mutations may impact the structure and function of centromeres and/or pericentric heterochromatin. A host of chromatin modifications that characterize centric heterochromatin can be examined, and a comparison of BRCA1 deficient breast cancer cells (e.g., human HCC1937) with normal control cells or BRCA1+ breast cancer cells can be used to show the effect of BRCA1 in centromere and heterochromatin structure and function. Chromatin modifications include biochemical hallmarks, such as lysK9, methK27, HP1, as well structural condensation and nuclear organization of centromeres.

We have found that centromeres are markedly ubiquitinated in a subset of cells, and we believe that BRCA1 (a ubiquitin ligase) plays a role in ubiquitination at the centromere, including Ub of Topo II and histone H2A. In addition, the loss of BRCA1 causes defects in mitotic chromosome segregation. BRCA1 status is believed to be linked to defective centromere segregation or microtubule association. DNA “bridges” seen in mitotic or early G1 cells lacking BRCA1 may be composed of centromeric satellite DNA. Other factors, in addition to known BRCA1-associated proteins or chromatin remodeling or DNA repair factors may localize with BRCA1 at constitutive heterochromatin.

BRCA1 is believed to function at chromosomal centromeres, structures critical for proper chromosome segregation. This constitutes a fundamentally new paradigm for how BRCA1 defects cause genomic stability and cancer.

### REFERENCES

Bantignies, F., Roure, V., Comet, I., Leblanc, B., Schuettengruber, B., Bonnet, J., Tixier, V., Mas, A., and Cavalli, G. (2011). Polycomb-dependent regulatory contacts between distant Hox loci in Drosophila. Cell 144, 214-226.
Bernardi, R., and Pandolfi, P. P. (2007). Structure, dynamics and functions of promyelocytic leukaemia nuclear bodies. Nat Rev Mol Cell Biol 8, 1006-1016.
Britten, R. J., and Kohne, D. E. (1968). Repeated sequences in DNA: Hundreds of thousands of copies of DNA sequences have been incorporated into the genomes of higher organisms. Science 161, 529-540.
Cadieux, B., Ching, T. T., VandenBerg, S. R., and Costello, J. F. (2006). Genome-wide hypomethylation in human glioblastomas associated with specific copy number alteration, methylenetetrahydrofolate reductase allele status, and increased proliferation. Cancer Res 66, 8469-8476.
Cindolo, L., Cantile, M., Vacherot, F., Terry, S., and de la Taille, A. (2007). Neuroendocrine differentiation in prostate cancer: from lab to bedside. Urol Int 79, 287-296.
Clemson, C. M., Hall, L. L., Byron, M., McNeil, J., and Lawrence, J. B. (2006). The X chromosome is organized into a gene-rich outer rim and an internal core containing silenced nongenic sequences. Proc Natl Acad Sci USA 103, 7688-7693.
Clemson, C. M., Hutchinson, J. N., Sara, S. A., Ensminger, A. W., Fox, A. H., Chess, A., and Lawrence, J. B. (2009). An architectural role for a nuclear noncoding RNA: NEAT1 RNA is essential for the structure of paraspeckles. Mol Cell 33, 717-726.
Ehrlich, M. (2009). DNA hypomethylation in cancer cells. Epigenomics 1, 239-259.
Eskeland, R., Leeb, M., Grimes, G. R., Kress, C., Boyle, S., Sproul, D., Gilbert, N., Fan, Y., Skoultchi, A. I., Wutz, A., et al. (2010). Ring1B compacts chromatin structure and represses gene expression independent of histone ubiquitination. Mol Cell 38, 452-464.
Fabiani, E., Leone, G., Giachelia, M., D'Alo, F., Greco, M., Criscuolo, M., Guidi, F., Rutella, S., Hohaus, S., and Voso, M. T. (2010). Analysis of genome-wide methylation and gene expression induced by 5-aza-2′-deoxycytidine identifies BCL2L10 as a frequent methylation target in acute myeloid leukemia. Leuk Lymphoma 51, 2275-2284.
Feinberg, A. P., and Tycko, B. (2004). The history of cancer epigenetics. Nat Rev Cancer 4, 143-153.
Fischer, A. H., Zhao, C., Li, Q. K., Gustafson, K. S., Eltoum, I. E., Tambouret, R., Benstein, B., Savaloja, L. C., and Kulesza, P. (2010). The cytologic criteria of malignancy. J Cell Biochem 110, 795-811.
Fraga, M. F., Ballestar, E., Villar-Garea, A., Boix-Chornet, M., Espada, J., Schotta, G., Bonaldi, T., Haydon, C., Ropero, S., Petrie, K., et al. (2005). Loss of acetylation at Lys16 and trimethylation at Lys20 of histone H4 is a common hallmark of human cancer. Nat Genet 37, 391-400.
Fraga, M. F., and Esteller, M. (2005). Towards the human cancer epigenome: a first draft of histone modifications. Cell Cycle 4, 1377-1381.
Glinsky, G. V. (2008). “Sternness” genomics law governs clinical behavior of human cancer: implications for decision making in disease management. J Clin Oncol 26, 2846-2853.
Gore, S. D., Baylin, S., Sugar, E., Carraway, H., Miller, C. B., Carducci, M., Greyer, M., Galm, O., Dauses, T., Karp, J. E., et al. (2006). Combined DNA methyltransferase and histone deacetylase inhibition in the treatment of myeloid neoplasms. Cancer Res 66, 6361-6369.
Grimaud, C., Negre, N., and Cavalli, G. (2006). From genetics to epigenetics: the tale of Polycomb group and trithorax group genes. Chromosome Res 14, 363-375.
Hall, L., and Lawrence, J. (2011). XIST RNA and Architecture of the Inactive X chromosome: Implications for the Repeat Genome. Cold Spring Harb Perspect Biol in press.
Hall, L. L., Byron, M., Sakai, K., Carrel, L., Willard, H. F., and Lawrence, J. B. (2002). An ectopic human XIST gene can induce chromosome inactivation in postdifferentiation human HT-1080 cells. Proc Natl Acad Sci USA 99, 8677-8682.
Hall, L. L., Smith, K. P., Byron, M., and Lawrence, J. B. (2006). Molecular anatomy of a speckle. Anat Rec A Discov Mol Cell Evol Biol 288, 664-675.
Hernandez-Munoz, I., Taghavi, P., Kuijl, C., Neefjes, J., and van Lohuizen, M. (2005). Association of BMI1 with polycomb bodies is dynamic and requires PRC2/EZH2 and the maintenance DNA methyltransferase DNMT1. Mol Cell Biol 25, 11047-11058.
Hite, K. C., Adams, V. H., and Hansen, J. C. (2009). Recent advances in MeCP2 structure and function. Biochem Cell Biol 87, 219-227.
Jacobs, J. J., Kieboom, K., Marino, S., DePinho, R. A., and van Lohuizen, M. (1999). The oncogene and Polycomb-group gene bmi-1 regulates cell proliferation and senescence through the ink4a locus. Nature 397, 164-168.
Jeanpierre, M. (1994). Human satellites 2 and 3. Ann Genet 37, 163-171.
Jeffery, L., and Nakielny, S. (2004). Components of the DNA methylation system of chromatin control are RNA-binding proteins. J Biol Chem 279, 49479-49487.
Ji, W., Hernandez, R., Zhang, X. Y., Qu, G. Z., Frady, A., Varela, M., and Ehrlich, M. (1997). DNA demethylation and pericentromeric rearrangements of chromosome 1. Mutat Res 379, 33-41.
Johnson, C. V., Singer, R. H., and Lawrence, J. B. (1991). Fluorescent detection of nuclear RNA and DNA: Implication for genome organization. Methods Cell Biol 35, 73-99.
Jolly, C., Metz, A., Govin, J., Vigneron, M., Turner, B. M., Khochbin, S., and Vourc'h, C. (2004). Stress-induced transcription of satellite III repeats. J Cell Biol 164, 25-33.
Jones, P. A., and Baylin, S. B. (2007). The epigenomics of cancer. Cell 128, 683-692.
Joulie, M., Miotto, B., and Defossez, P. A. (2010). Mammalian methyl-binding proteins: what might they do? Bioessays 32, 1025-1032.
Kanadia, R. N., Johnstone, K. A., Mankodi, A., Lungu, C., Thornton, C. A., Esson, D., Timmers, A. M., Hauswirth, W. W., and Swanson, M. S. (2003). A muscleblind knockout model for myotonic dystrophy. Science 302, 1978-1980.
Kelly, T. K., De Carvalho, D. D., and Jones, P. A. (2010). Epigenetic modifications as therapeutic targets. Nat Biotechnol 28, 1069-1078.
Koziol, M. J., and Rinn, J. L. (2010). RNA traffic control of chromatin complexes. Curr Opin Genet Dev 20, 142-148.
Langmead, B., Trapnell, C., Pop, M., & Salzberg, S. L. (2009). Ultrafast and memory-efficient alignment of short DNA sequences to the human genome. Genome Biology, 10(3), R25. doi:10.1186/gb-2009-10-3-r25.
Long, S. W., Ooi, J. Y., Yau, P. M., and Jones, P. L. (2010). A brain-derived mecp2 complex supports a role for MeCP2 in RNA processing. Biosci Rep.
Lu, J., and Gilbert, D. M. (2007). Proliferation-dependent and cell cycle regulated transcription of mouse pericentric heterochromatin. J Cell Biol 179, 411-421.
Lukacs, R. U., Memarzadeh, S., Wu, H., and Witte, O. N. (2010). Bmi-1 is a crucial regulator of prostate stem cell self-renewal and malignant transformation. Cell Stem Cell 7, 682-693.
Masui, O., and Heard, E. (2006). RNA and protein actors in X-chromosome inactivation. Cold Spring Harb Symp Quant Biol 71, 419-428.
Mertens, F., Johansson, B., Hoglund, M., and Mitelman, F. (1997). Chromosomal imbalance maps of malignant solid tumors: a cytogenetic survey of 3185 neoplasms. Cancer Res 57, 2765-2780.
Misteli, T. (2000). Cell biology of transcription and pre-mRNA splicing: nuclear architecture meets nuclear function. J Cell Sci 113, 1841-1849.
Misteli, T. (2004). Spatial positioning; a new dimension in genome function. Cell 119, 153-156.
Motorin, Y., Lyko, F., and Helm, M. (2010). 5-methylcytosine in RNA: detection, enzymatic formation and biological functions. Nucleic Acids Res 38, 1415-1430.
Niessen, H. E., Demmers, J. A., and Voncken, J. W. (2009). Talking to chromatin: post-translational modulation of polycomb group function. Epigenetics Chromatin 2, 10.
Osborne, R. J., and Thornton, C. A. (2006). RNA-dominant diseases. Hum Mol Genet 15 Spec No 2, R162-169.
Pageau, G1, Hall, L. L., Ganesan, S., Livingston, D. M., and Lawrence, J. B. (2007). The disappearing Barr body in breast and ovarian cancers. Nat Rev Cancer 7, 628-633.
Probst, A. V., and Almouzni, G. (2007). Pericentric heterochromatin: dynamic organization during early development in mammals. Differentiation.
Prosser, J., Frommer, M., Paul, C., and Vincent, P. C. (1986). Sequence relationships of three human satellite DNAs. J Mol Biol 187, 145-155.
Richard, G. F., Kerrest, A., and Dujon, B. (2008). Comparative genomics and molecular dynamics of DNA repeats in eukaryotes. Microbiol Mol Biol Rev 72, 686-727.
Riis, M. L., Luders, T., Nesbakken, A. J., Vollan, H. S., Kristensen, V., and Bukholm, I. R. (2010). Expression of BMI-1 and Mel-18 in breast tissue—a diagnostic marker in patients with breast cancer. BMC Cancer 10, 686.
Rizzi, N., Denegri, M., Chiodi, I., Corioni, M., Valgardsdottir, R., Cobianchi, F., Riva, S., and Biamonti, G. (2004). Transcriptional activation of a constitutive heterochromatic domain of the human genome in response to heat shock. Mol Biol Cell 15, 543-551.
Saurin, A. J., Shiels, C., Williamson, J., Satijn, D. P., Otte, A. P., Sheer, D., and Freemont, P. S. (1998). The human polycomb group complex associates with pericentromeric heterochromatin to form a novel nuclear domain. J Cell Biol 142, 887-898.
Silahtaroglu, A., Pfundheller, H., Koshkin, A., Tommerup, N., and Kauppinen, S. (2004). LNA-modified oligonucleotides are highly efficient as FISH probes. Cytogenet Genome Res 107, 32-37.
Smith, K., Byron, M., Johnson, C., Xing, Y., and Lawrence, J. B. (2007). Defining early steps in mRNA transport: Mutant mRNA in Myotonic Dystrophy Type I is blocked at entry into SC-35 domains. Journal of Cell Biology In Press.
Sparmann, A., and van Lohuizen, M. (2006). Polycomb silencers control cell fate, development and cancer. Nat Rev Cancer 6, 846-856.
Spector, D. L. (2006). SnapShot: Cellular bodies. Cell 127, 1071.
Tam, R., Shopland, L. S., Johnson, C. V., McNeil, J., and Lawrence, J. B. (2002). Applications of RNA FISH for visualizing gene expression and nuclear architecture”, Vol 260 (New York, Oxford University Press).
Ting, D. T., Lipson, D., Paul, S., Brannigan, B. W., Akhavanfard, S., Coffman, E. J., Contino, G.,
Deshpande, V., Iafrate, A. J., Letovsky, S., et al. (2011). Aberrant overexpression of satellite repeats in pancreatic and other epithelial cancers. Science 331, 593-596.
Valk-Lingbeek, M. E., Bruggeman, S. W., and van Lohuizen, M. (2004). Stem cells and cancer; the polycomb connection. Cell 118, 409-418.
Voncken, J. W., Schweizer, D., Aagaard, L., Sattler, L., Jantsch, M. F., and van Lohuizen, M. (1999). Chromatin-association of the Polycomb group protein BMI1 is cell cycle-regulated and correlates with its phosphorylation status. J Cell Sci 112 (Pt 24), 4627-4639.
Vourc'h, C., and Biamonti, G. (2011). Transcription of Satellite DNAs in Mammals. Prog Mol Subcell Biol 51, 95-118.
Wilusz, J. E., Sunwoo, H., and Spector, D. L. (2009). Long noncoding RNAs: functional surprises from the RNA world. Genes Dev 23, 1494-1504.
Xing, Y., Johnson, C. V., Moen, P. T., McNeil, J. A., and Lawrence, J. B. (1995). Nonrandom gene organization: Structural arrangements of specific pre-mRNA transcription and splicing with SC-35 domains. J Cell Biol 131, 1635-1647.
Yildirim, O., Li, R., Hung, J.-H., Chen, P. B., Dong, X., Ee, L.-S., Weng, Z., et al. (2011). Mbd3/NURD Complex Regulates Expression of 5-Hydroxymethylcytosine Marked Genes in Embryonic Stem Cells. Cell, 147(7), 1498-1510. doi:10.1016/j.cell.2011.11.054.
Young, J. I., Hong, E. P., Castle, J. C., Crespo-Barreto, J., Bowman, A. B., Rose, M. F., Kang, D., Richman, R., Johnson, J. M., Berget, S., et al. (2005). Regulation of RNA splicing by the methylation-dependent transcriptional repressor methyl-CpG binding protein 2. Proc Natl Acad Sci USA 102, 17551-17558.
Zagradisnik, B., and Kokalj-Vokac, N. (2000). Hypomethylation of alphoid DNA and classical satellite DNA on chromosome 1, 9, 16 and Yin extraembryonic tissue. Pflugers Arch 440, R190-192.

### Other Embodiments

All publications, patents, and patent applications mentioned in the above specification are hereby incorporated by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of diagnosing, or providing a prognostic indicator of, cancer in a mammal comprising detecting a biomarker selected from a satellite II ribonucleic acid (RNA) molecule, a cancer-associated polycomb group (CAP) body, and a cancer-associated satellite transcript (CAST) body in a sample from said mammal.

2. The method of claim 1, wherein an increase in the level of expression of said satellite II RNA molecule in a cell of said sample, relative to the level of expression of said satellite II RNA molecule in a normal cell, or abnormal nuclear compartmentalization of said CAP body or said CAST body in a cell of said sample, relative to nuclear compartmentalization of said CAP body or said CAST body in a normal cell, indicates said sample comprises a cancer cell

3. The method of claim 1, wherein said CAP body comprises a satellite II deoxyribonucleic acid (DNA) molecule or one or more polycomb group proteins.

4. The method of claim 3, wherein said polycomb group proteins are selected from one or more of a polycomb-repressive complex 1 (PRC1) protein selected from one or more of BMI-1, RING 1B, Phc1, Phc2, CBX4, CBX8, and RNF2, a polycomb-repressive complex 2 (PRC2) protein selected from one or more of SUZ12, EED, RBBP4, JARID2, EZH2, EZH1, and RBBP7, and a PRC1 complex-interacting protein selected from one or more of GLI1, MYC, CDKN2A, and HST2H2AC.

5. The method of claim 1, wherein said CAST body comprises said satellite II ribonucleic acid (RNA) molecule.

6. The method of claim 1, wherein said CAST body comprises a protein selected from methyl CpG (cytosine phosphate guanine) binding protein 2 (MeCP2), SIN3A, CDKL5, DNMT1, HDAC1, ATRX, DNMT3B, SMARCA2, DLX5, BDNF, UBE3A, MBNL 1, MBNL 2, MBNL 3, hnRNP H, hnRNP G, hnRNP A, hnRNP K, proteosome 20Sαsubunit, proteosome 11Sγsubunit, proteosome 11sα subunit, Y12, Y14, 9G8, snRNP Sm antigen, SAM68, SLM 1 and 2, Tra2β, Purα, and CPEB protein.

7. The method of claim 1, wherein said method comprises detecting the distribution, level, or presence of said biomarker in at least one cell of said sample using radioimmunoassay (RIA), enzyme-linked immunosorbent assay (ELISA), immunoblotting, immunoprecipitation, or microscopy.

8. The method of claim 7, wherein said immuniprecipitation is chromatin immunoprecipitation, wherein said method comprises digesting the genome of said cell in the sample, contacting an antibody that specifically binds one or more proteins of said CAP body to said digested genome in the sample, separating an antibody/CAP body/chromatin complex comprising DNA from the sample, and sequencing the DNA from the antibody/CAP body/chromatin complex, wherein an increased presence of a satellite II DNA sequence within the antibody/CAP body/chromatin complex indicates the sample comprises said cancer cell.

9. The method of claim 7, wherein said immunoprecipitation comprises digesting the genome of said cell in the sample, contacting a nucleic acid molecule complementary to and specific for a satellite II DNA sequence to said digested genome to form a hybridization complex, separating said hybridization complex from the sample, and contacting one or more components of said hybridization complex with an antibody that specifically binds to one or more proteins of said CAP body, wherein binding of said antibody to one or more of said proteins of said CAP body indicates the sample comprises said cancer cell.

10. The method of claim 1, wherein said method comprises detecting said satellite II RNA molecule in said sample using a method selected from a microarray, RNA fluorescence in situ hybridization (FISH), northern blot, polymerase chain reaction (PCR), RNA sequencing, and microscopy.

11. The method of claim 3, wherein said method comprises detecting said satellite II DNA molecule in said sample using a method selected from a microarray, DNA fluorescence in situ hybridization (FISH), Southern blot, polymerase chain reaction (PCR), and DNA sequencing.

12. The method of claim 1, wherein said biomarker is detected with an antibody that binds a polycomb group protein of said CAP body selected from BMI-1, RING 1B, Phc1, Phc2, CBX4, CBX8, RNF2, SUZ12, EED, RBBP4, JARID2, EZH2, EZH1, RBBP7, GLI1, MYC, CDKN2A, and HST2H2AC, or a protein of said CAST body selected from MeCP2, SIN3A, CDKL5, DNMT1, HDAC1, ATRX, DNMT3B, SMARCA2, DLX5, BDNF, UBE3A, MBNL 1, MBNL 2, MBNL 3, hnRNP H, hnRNP G, hnRNP A, hnRNP K, proteosome 20Sαsubunit, proteosome 11Sαsubunit, proteosome 11sγ subunit, Y12, Y14, 9G8, snRNP Sm antigen, SAM68, SLM 1 and 2, Tra2β, Purα, and CPEB protein.

13. The method of claim 1, wherein said satellite II RNA molecule is detected using a probe comprising a sequence having at least 80% sequence identity to the sequence of any one of SEQ ID NOs: 2 to 10, or its complement, or a probe comprising a sequence having at least 80% sequence identity to a sequence comprising at least 20 consecutive nucleotides of any one of SEQ ID NOs: 14 to 28.

14. The method of claim 1, wherein the sample comprises an organ, tissue, skin, hair, fecal matter, cell, bodily fluid, or lavage from said mammal.

15. The method of claim 14, wherein said bodily fluid is selected from saliva, serum, plasma, blood, urine, mucus, gastric juices, pancreatic juices, semen, products of lactation or menstruation, tears, and lymph, or wherein said lavage is selected from a bronchalveolar lavage, a gastric lavage, a peritoneal lavage, a vaginal lavage, a colonic or rectal lavage, an arthroscopic lavage, a ductal lavage, and an ear lavage.

16. The method of claim 1, wherein said cancer is metastatic cancer or a cancer selected from breast cancer, ovarian cancer, Wilms tumor, multiple myeloma, brain cancer, kidney cancer, lung cancer, fibrosarcoma, prostate cancer, stomach cancer, thyroid cancer, bone cancer, colon cancer, pancreatic cancer, and cervical cancer.

17. The method of claim 1, wherein said mammal is a human.

18. A method for identifying an agent for the treatment of a cancer in a mammal comprising contacting a cancer cell comprising a biomarker selected from a cancer-associated polycomb group (CAP) body, a cancer-associated satellite transcript (CAST) body, and a satellite II RNA molecule with a test agent and determining whether the test agent reduces the level of the biomarker by detecting a reduction in the formation of the CAP body or CAST body, or a reduction in expression of the satellite II RNA molecule, in said cancer cell, wherein a reduction in the level of the biomarker in said cancer cell, relative to the level of the biomarker in a cancer cell not contacted with the test agent, indicates that the test agent is suitable for the treatment of the cancer.

19. A method for determining whether a chemotherapeutic agent increases epigenetic imbalance in a cell of a mammal comprising contacting said cell with a chemotherapeutic agent and determining a level of a biomarker selected from a cancer-associated polycomb group (CAP) body, a cancer-associated satellite transcript (CAST) body, and a satellite II RNA molecule in said cell, wherein an increase in the level of the biomarker in said cell, relative to the level of the biomarker in a cell not contacted with the chemotherapeutic agent, indicates that the chemotherapeutic agent increases epigenetic imbalance in said cell, wherein said epigenetic imbalance is associated with an increased risk of cancer in said mammal.

20-22. (canceled)

23. The method of claim 1, wherein said CAP body is present at the 1q12 or 16q11 DNA locus.

24-25. (canceled)

26. The method of claim 5, wherein said satellite II RNA molecule is cytosine methylated.

27. The method of claim 1, wherein said CAST body comprises a methyl DNA binding protein.

28. The method of claim 27, wherein said methyl DNA binding protein is methyl CpG (cytosine phosphate guanine) binding protein 2 (MeCP2).

29-48. (canceled)

49. A method for detecting epigenetic imbalance in a cell of a mammal comprising determining a copy number of, or the level of polycomb proteins on, a satellite II DNA locus at chromosome 1q12 in said cell, wherein an increase in said copy number of, or an increase in the number of said polycomb proteins on, said satellite II DNA locus, relative to a non-cancer control cell, indicates said cell has said epigenetic imbalance wherein said epigenetic imbalance indicates an increased risk of cancer in said mammal.

50. (canceled)

51. A method for diagnosing, or providing a prognostic indicator of, cancer comprising detecting, as a biomarker, the ubiquitination status of histone H2A in a cell of a mammal, wherein the detection of an increase in UbH2A foci in said cell, relative to UbH2A foci in a non-cancer control cell, indicates the presence of cancer in the mammal; or detecting, as a biomarker, the distribution of a heterochromatic marker in a cell of the mammal, wherein an unbalanced distribution of the heterochromatic marker in the cell, relative to a non-cancer control cell, indicates the presence of cancer in the mammal.

52-57. (canceled)
